SEQUENCE LISTING (66 sequences)

SEQ ID NO: 1 - protein, 4 aa, Artificial Sequence
  Synthetic Construct
VPGG

SEQ ID NO: 2 - protein, 4 aa, Artificial Sequence
  Synthetic Construct
IPGG

SEQ ID NO: 3 - protein, 5 aa, Artificial Sequence
  Synthetic Construct
VPGXG

SEQ ID NO: 4 - protein, 5 aa, Artificial Sequence
  Synthetic Construct
AVGVP

SEQ ID NO: 5 - protein, 5 aa, Artificial Sequence
  Synthetic Construct
IPGVG

SEQ ID NO: 6 - protein, 5 aa, Artificial Sequence
  Synthetic Construct
LPGVG

SEQ ID NO: 7 - protein, 6 aa, Artificial Sequence
  Synthetic Construct
VAPGVG

SEQ ID NO: 8 - protein, 8 aa, Artificial Sequence
  Synthetic Construct
GVGVPGVG

SEQ ID NO: 9 - protein, 9 aa, Artificial Sequence
  Synthetic Construct
VPGFGVGAG

SEQ ID NO: 10 - protein, 9 aa, Artificial Sequence
  Synthetic Construct
VPGVGVPGG

SEQ ID NO: 11 - protein, 5 aa, Artificial Sequence
  Synthetic Construct
IPGXG

SEQ ID NO: 12 - protein, 5 aa, Artificial Sequence
  Synthetic Construct
LPGXG

SEQ ID NO: 13 - protein, 50 aa, Artificial Sequence
  Synthetic Construct
VPGVGVPGVGVPGVGVPGVGVPGVGVPGAGVPGAGVPGGGVPGGGVPGGG

SEQ ID NO: 14 - protein, 25 aa, Artificial Sequence
  Synthetic Construct
VPGVGVPGVGVPGVGVPGVGVPGVG

SEQ ID NO: 15 - DNA, 75 bp, Artificial Sequence
  Synthetic Construct
gtgggtgttccgggcgtaggtgtcccaggtgtgggcgtaccgggcgttggtgttcctggtgtcggcgtgccgggc

SEQ ID NO: 16 - protein, 450 aa, Artificial Sequence
  Synthetic Construct
VPGVGVPGVGVPGVGVPGVGVPGVGVPGAGVPGAGVPGGGVPGGGVPGGGVPGVGVPGVGVPGVGVPGVGVPGVGVPGAGVPGAGVPGGGVPGGGVPGGGVPGVGVPGVGVPGVGVPGVGVPGVGVPGAGVPGAGVPGGGVPGGGVPGGGVPGVGVPGVGVPGVGVPGVGVPGVGVPGAGVPGAGVPGGGVPGGGVPGGGVPGVGVPGVGVPGVGVPGVGVPGVGVPGAGVPGAGVPGGGVPGGGVPGGGVPGVGVPGVGVPGVGVPGVGVPGVGVPGAGVPGAGVPGGGVPGGGVPGGGVPGVGVPGVGVPGVGVPGVGVPGVGVPGAGVPGAGVPGGGVPGGGVPGGGVPGVGVPGVGVPGVGVPGVGVPGVGVPGAGVPGAGVPGGGVPGGGVPGGGVPGVGVPGVGVPGVGVPGVGVPGVGVPGAGVPGAGVPGGGVPGGGVPGGG

SEQ ID NO: 17 - protein, 100 aa, Artificial Sequence
  Synthetic Construct
VPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVG

SEQ ID NO: 18 - protein, 20 aa, Artificial Sequence
  Synthetic Construct
VPGKGVPGVGVPGVGVPGFG

SEQ ID NO: 19 - protein, 45 aa, Artificial Sequence
  Synthetic Construct
VPGKGVPGVGVPGVGVPGVGVPGVGVPGVGVPGVGVPGVGVPGFG

SEQ ID NO: 20 - protein, 25 aa, Artificial Sequence
  Synthetic Construct
AVGVPAVGVPAVGVPAVGVPAVGVP

SEQ ID NO: 21 - protein, 25 aa, Artificial Sequence
  Synthetic Construct
IPGVGIPGVGIPGVGIPGVGIPGVG

SEQ ID NO: 22 - protein, 25 aa, Artificial Sequence
  Synthetic Construct
LPGVGLPGVGLPGVGLPGVGLPGVG

SEQ ID NO: 23 - protein, 339 aa, Artificial Sequence
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASSGTDDDDKGIVEQCCTSICSLYQLENYCN

SEQ ID NO: 24 - protein, 489 aa, Artificial Sequence
  Synthetic Construct
MGGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPGASSGTDDDDKGIVEQCCTSICSLYQLENYCN

SEQ ID NO: 25 - protein, 639 aa, Artificial Sequence
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASSGTDDDDKGIVEQCCTSICSLYQLENYCN

SEQ ID NO: 26 - protein, 939 aa, Artificial Sequence
  Synthetic Construct
MGGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPGASSGTDDDDKGIVEQCCTSICSLYQLENYCN

SEQ ID NO: 27 - protein, 354 aa, Artificial Sequence
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASSGTDDDDKYTSLIHSLIEESQNQQEKNEQELLELDKWASLWNWF

SEQ ID NO: 28 - protein, 504 aa, Artificial Sequence
  Synthetic Construct
MGGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPGASSGTDDDDKYTSLIHSLIEESQNQQEKNEQELLELDKWASLWNWF

SEQ ID NO: 29 - protein, 654 aa, Artificial Sequence
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASSGTDDDDKYTSLIHSLIEESQNQQEKNEQELLELDKWASLWNWF

SEQ ID NO: 30 - protein, 357 aa, Artificial Sequence
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASGGGGPLVPRGSYTSLIHSLIEESQNQQEKNEQELLELDKWASLWNWF

SEQ ID NO: 31 - protein, 507 aa, Artificial Sequence
  Synthetic Construct
MGGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPGASGGGGPLVPRGSYTSLIHSLIEESQNQQEKNEQELLELDKWASLWNWF

SEQ ID NO: 32 - protein, 657 aa, Artificial Sequence
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASGGGGPLVPRGSYTSLIHSLIEESQNQQEKNEQELLELDKWASLWNWF

SEQ ID NO: 33 - protein, 357 aa, Artificial Sequence
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASGPTTENLYFQSYTSLIHSLIEESQNQQEKNEQELLELDKWASLWNWF

SEQ ID NO: 34 - protein, 507 aa, Artificial Sequence
  Synthetic Construct
MGGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPGASGPTTENLYFQSYTSLIHSLIEESQNQQEKNEQELLELDKWASLWNWF

SEQ ID NO: 35 - protein, 657 aa, Artificial Sequence
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASGPTTENLYFQSYTSLIHSLIEESQNQQEKNEQELLELDKWASLWNWF

SEQ ID NO: 36 - protein, 356 aa, Artificial Sequence
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASGPTTENLYFQYTSLIHSLIEESQNQQEKNEQELLELDKWASLWNWF

SEQ ID NO: 37 - protein, 506 aa, Artificial Sequence
  Synthetic Construct
MGGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPGASGPTTENLYFQYTSLIHSLIEESQNQQEKNEQELLELDKWASLWNWF

SEQ ID NO: 38 - protein, 656 aa, Artificial Sequence
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASGPTTENLYFQYTSLIHSLIEESQNQQEKNEQELLELDKWASLWNWF

SEQ ID NO: 39 - protein, 669 aa, Artificial Sequence
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGLVPRGSPGISGGGGGHMPMALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 40 - protein, 574 aa, Artificial Sequence
  Synthetic Construct
MRALMGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPSSGLVPRGSPGISGGGGGHMPMGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNELVYSQ

SEQ ID NO: 41 - protein, 724 aa, Artificial Sequence
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGLVPRGSPGISGGGGGHMPMGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNELVYSQ

SEQ ID NO: 42 - protein, 874 aa, Artificial Sequence
  Synthetic Construct
MRALMGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPSSGLVPRGSPGISGGGGGHMPMGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNELVYSQ

SEQ ID NO: 43 - protein, 1174 aa, Artificial Sequence
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGLVPRGSPGISGGGGGHMPMGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNELVYSQ

SEQ ID NO: 44 - protein, 735 aa, Artificial Sequence
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGGGSIGPLVPRGSHMSTSQPGACPCQGAASRPAILYALLSSSLKAVPRPRSRCLCRQHRPVQLCAPHRTCREALDVLAKTVAFLRNLPSFWQLPPQDQRRLLQGCWGPLFLLGLAQDAVTFEVAEAPVPSILKKILLEEPSSSGGSGQLPDRPQPSLAAVQWLQCCLESFWSLELSPKEYACLKGTILFNPDVPGLQAASHIGHLQQEAHWVLCEVLEPWCPAAQGRLTRVLLTASTLKSIPTSLLGDLFFRPIIGDVDIAGLLGDMLLLR

SEQ ID NO: 45 - protein, 736 aa, Artificial Sequence
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGGGSIGPLVPRGSHMHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 46 - protein, 1186 aa, Artificial Sequence
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGGGSIGPLVPRGSHMHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 47 - protein, 757 aa, Artificial Sequence
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGGGSIGPLVPRGSHMIQQATTGVSQETSENPGDKTIVPATLPQLTPTLVSLLEVIEPEVLYAGYDSSVPDSTWRIMTTLNMLGGRQVIAAVKWAKAIPGFRNLHLDDQMTLLQYSWMSLMAFALGWRSYRQSSANLLCFAPDLIINEQRMTLPDMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 48 - protein, 624 aa, Artificial Sequence
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGGGSIGPLVPRGSHMSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 49 - protein, 774 aa, Artificial Sequence
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGGGSIGPLVPRGSHMSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 50 - protein, 1225 aa, Artificial Sequence
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGLVPRGSPGISGGGGGHMSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 51 - protein, 775 aa, Artificial Sequence
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGDYDIPTTENLYFQGAHMSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 52 - protein, 859 aa, Artificial Sequence
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGSIGPLVPRGSHSMGLNDIFEAQKIEWHEHMPMALEMTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKINYSHFTCATDTENIRFVFAAVKDTILQLNLKEYNLV

SEQ ID NO: 53 - protein, 1309 aa, Artificial Sequence
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGSIGPLVPRGSHSMGLNDIFEAQKIEWHEHMPMALEMTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKINYSHFTCATDTENIRFVFAAVKDTILQLNLKEYNLV

SEQ ID NO: 54 - protein, 728 aa, Artificial Sequence
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGGGSIGPLVPRGSHMKQLTILGSTGSIGCSTLDVVRHNPEHFRVVALVAGKNVTRMVEQCLEFSPRYAVMDDEASAKLLKTMLQQQGSRTEVLSGQQAACDMAALEDVDQVMAAIVGAAGLLPTLAAIRAGKTILLANKESLVTCGRLFMDAVKQSKAQLLPVDSEHNAIFQSLPQPIQHNLGYADLEQNGVVSILLTGSGGPFRETPLRDLATMTPDQACRHPNWSMGRKISVDSATMMNKGLEYIEARWLFNASASQMEVLIHPQSVIHSMVRYQDGSVLAQLGEPDMRTPIAHTMAWPNRVNSGVKPLDFCKLSALTFAAPDYDRYPCLKLAMEAFEQGQAATTALNAANEITVAAFLAQQIRFTDIAALNLSVLEKMDMREPQCVDDVLSVDASAREVARKEVMRLASPV

SEQ ID NO: 55 - protein, 879 aa, Artificial Sequence
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGLVPRGSPGISGGGGGHMKQLTILGSTGSIGCSTLDVVRHNPEHFRVVALVAGKNVTRMVEQCLEFSPRYAVMDDEASAKLLKTMLQQQGSRTEVLSGQQAACDMAALEDVDQVMAAIVGAAGLLPTLAAIRAGKTILLANKESLVTCGRLFMDAVKQSKAQLLPVDSEHNAIFQSLPQPIQHNLGYADLEQNGVVSILLTGSGGPFRETPLRDLATMTPDQACRHPNWSMGRKISVDSATMMNKGLEYIEARWLFNASASQMEVLIHPQSVIHSMVRYQDGSVLAQLGEPDMRTPIAHTMAWPNRVNSGVKPLDFCKLSALTFAAPDYDRYPCLKLAMEAFEQGQAATTALNAANEITVAAFLAQQIRFTDIAALNLSVLEKMDMREPQCVDDVLSVDASAREVARKEVMRLASPV

SEQ ID NO: 56 - protein, 1329 aa, Artificial Sequence
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGLVPRGSPGISGGGGGHMKQLTILGSTGSIGCSTLDVVRHNPEHFRVVALVAGKNVTRMVEQCLEFSPRYAVMDDEASAKLLKTMLQQQGSRTEVLSGQQAACDMAALEDVDQVMAAIVGAAGLLPTLAAIRAGKTILLANKESLVTCGRLFMDAVKQSKAQLLPVDSEHNAIFQSLPQPIQHNLGYADLEQNGVVSILLTGSGGPFRETPLRDLATMTPDQACRHPNWSMGRKISVDSATMMNKGLEYIEARWLFNASASQMEVLIHPQSVIHSMVRYQDGSVLAQLGEPDMRTPIAHTMAWPNRVNSGVKPLDFCKLSALTFAAPDYDRYPCLKLAMEAFEQGQAATTALNAANEITVAAFLAQQIRFTDIAALNLSVLEKMDMREPQCVDDVLSVDASAREVARKEVMRLASPV

SEQ ID NO: 57 - protein, 879 aa, Artificial Sequence
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGDYDIPTTENLYFQGAHMKQLTILGSTGSIGCSTLDVVRHNPEHFRVVALVAGKNVTRMVEQCLEFSPRYAVMDDEASAKLLKTMLQQQGSRTEVLSGQQAACDMAALEDVDQVMAAIVGAAGLLPTLAAIRAGKTILLANKESLVTCGRLFMDAVKQSKAQLLPVDSEHNAIFQSLPQPIQHNLGYADLEQNGVVSILLTGSGGPFRETPLRDLATMTPDQACRHPNWSMGRKISVDSATMMNKGLEYIEARWLFNASASQMEVLIHPQSVIHSMVRYQDGSVLAQLGEPDMRTPIAHTMAWPNRVNSGVKPLDFCKLSALTFAAPDYDRYPCLKLAMEAFEQGQAATTALNAANEITVAAFLAQQIRFTDIAALNLSVLEKMDMREPQCVDDVLSVDASAREVARKEVMRLASPV

SEQ ID NO: 58 - protein, 864 aa, Artificial Sequence
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGGGSIGPLVPRGSHMPMALEMGCLGNSKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 59 - protein, 11 aa, Artificial Sequence
  Synthetic construct
VENLYFQGGMG

SEQ ID NO: 60 - protein, 24 aa, Artificial Sequence
  Synthetic construct
VPGWPSSGDYDIPTTENLYFQGAH

SEQ ID NO: 61 - protein, 24 aa, Artificial Sequence
  Synthetic construct
GSGSGHMHHHHHHSSGLVPRGSGK

SEQ ID NO: 62 - protein, 24 aa, Artificial Sequence
  Synthetic construct
VPGWPSSGDYDIPTTENLYFQGAH

SEQ ID NO: 63 - protein, 27 aa, Artificial Sequence
  Synthetic construct
VDKLAAALDMHHHHHHSSGLVPRGSGK

SEQ ID NO: 64 - protein, 24 aa, Artificial Sequence
  Synthetic construct
VPGWPSSGDYDIPTTENLYFQGAH

SEQ ID NO: 65 - protein, 11 aa, Artificial Sequence
  Synthetic construct
LENLYFQGGMG

SEQ ID NO: 66 - protein, 24 aa, Artificial Sequence
  Synthetic construct
VPGWPSSGDYDIPTTENLYFQGAH

(19) United States
(12) Patent Application Publication
(10) Pub. No.: US 2011/0039776 A1
(43) Pub. Date: Feb. 17, 2011

(54) FUSION PEPTIDE THERAPEUTIC COMPOSITIONS

(76) Inventor: Ashutosh Chilkoti, Durham, NC (US)

Correspondence Address: COOLEY LLP;ATTN: Patent Group, Suite 1100, 777 - 6th Street, NW, WASHINGTON, DC, 20001, US

(21) Appl. No.: 12/440,294

(22) PCT Filed: Sep. 6, 2007
(86) PCT No.: PCT/US07/77767

§ 371 Date: Aug. 4, 2010

Related U.S. Application Data
(60) Provisional application No. 60/842,464, filed on Sep. 6, 2006.

(51) Int. Cl. A61K 38/26

(52) U.S. Cl. 514/11.7

(57) ABSTRACT

Therapeutic compositions containing fusion proteins (FPs) including elastin-like peptides (ELPs) and peptide active therapeutic agents, and methods of making and using such compositions and fusion proteins. Therapeutic compositions of such type enable improved efficacy of the peptide active therapeutic agent to be achieved, in relation to the peptide active therapeutic agent alone. Enhanced efficacy of the peptide active therapeutic agent in the therapeutic composition may include improved solubility, bioavailability, bio-unavailability, half-life, etc., as compared to corresponding compositions containing the same peptide active therapeutic agent without associated ELPs.

### RELATED APPLICATION DATA

The application claims priority under 35 U.S.C. §119(e) to U.S. Patent application Ser. No. 60/842,464, filed Sep. 6, 2006, incorporated by reference herein in its entirety.

### FIELD OF THE INVENTION

The invention provides a new generation of therapeutic compositions, incorporating fusion proteins (FPs) including elastin-like peptides (ELPs) and peptide active therapeutic agents. The therapeutic compositions of the invention enable improved solubility, bioavailability or bio-unavailability, and half-life of the administered peptide active therapeutic agents to be achieved, as compared to corresponding compositions containing the same peptide active therapeutic agents without associated ELPs.

### BACKGROUND OF THE INVENTION

The disclosures of U.S. Pat. No. 6,852,834, issued Feb. 8, 2005 in the name of Ashutosh Chilkoti for “FUSION PEPTIDES ISOLATABLE BY PHASE TRANSITION,” and U.S. patent application Ser. No. 11/053,100 filed Feb. 8, 2005 in the name of Ashutosh Chilkoti for “FUSION PEPTIDES ISOLATABLE BY PHASE TRANSITION,” are hereby incorporated herein by reference, in their respective entireties, for all purposes.

The aforementioned Chilkoti patent and patent application disclose genetically-encodable, environmentally-responsive fusion proteins comprising ELP peptides. Such fusion proteins exhibit unique physico-chemical and functional properties that can be modulated as a function of solution environment.

### SUMMARY OF THE INVENTION

The present invention relates broadly to fusion protein (FP) therapeutic compositions including elastin-like peptides (ELPs) and peptide active therapeutic agents.

In the FP therapeutic compositions of the invention, at least one peptide active therapeutic agent is coupled to one or more ELPs, e.g., being covalently bonded at an N- or C-terminus thereof, to achieve enhancement of the efficacy of the peptide active therapeutic agent(s), in relation to the corresponding therapeutic agent(s) alone. The peptide active therapeutic agent-ELP construct has enhanced efficacy in respect of any of various properties, such as solubility, bioavailability, bio-unavailability, therapeutic dose, resistance to proteolysis, half-life of the administered peptide active therapeutic agent, etc.

In another aspect, the invention relates to fusion gene constructs, including heterologous nucleotide sequences operably linked to an expression control element, e.g., a promoter of appropriate type, wherein the heterologous nucleotide sequences encode a fusion protein including at least one peptide active therapeutic agent coupled to at least one ELP.

In a further aspect, the invention relates to a method of enhancing efficacy of a peptide active therapeutic agent. The method includes coupling the peptide active therapeutic agent with at least one ELP to form a FP therapeutic composition, wherein the peptide active therapeutic agent in such FP therapeutic composition has enhanced efficacy, in relation to the peptide active therapeutic agent alone. In one aspect the enhanced efficacy is in vivo efficacy.

Another aspect of the invention relates to a method of treating a subject in need of a peptide active therapeutic agent, including administering to the patient a therapeutic composition including: (i) the peptide active therapeutic agent to coupled with at least one ELP, or (ii) a nucleotide sequence encoding a fusion protein including the peptide active therapeutic agent and at least one ELP, operably linked to an expression control element therefore.

In still another aspect, the invention relates to a therapeutic agent dose form, in which the therapeutic agent is conjugated with an ELP.

Various other aspects, features and embodiments of the invention will be more fully apparent from the ensuing disclosure and appended claims.

### BRIEF DESCRIPTION OF THE FIGURES

FIG. 1 is an SDS-PAGE gel showing expression of a 37 amino acid peptide, using the expression and purification methods of Example 1.

FIG. 2 is a graph confirming the purification of the peptides resulting from the methods of Example 1.

FIG. 3 is an SDS-PAGE gel showing the results of ITC purification of BFP, CAT and K1-3, as set forth in Example 6.

FIGS. 4A and 4B are graphs of the increase in turbidity as a function of temperature of each of the fusion constructs of Example 8 in PBS buffer.

FIG. 5 is graph illustrating the blood concentration time-course for ¹⁴C labeled ELP, as set forth in Example 9.

FIG. 6 is a graph showing biodistribution of ¹⁴C labeled ELP1-150 and ELP 2-160 in nude mice, as described in Example 10.

FIG. 7 is a graph showing biodistribution of ¹⁴C labeled ELP2-[V₁A₈G₇-160] in nude mice, as described in Example 10.

### DETAILED DESCRIPTION OF THE INVENTION

The invention provides therapeutic compositions incorporating fusion proteins (FPs) including elastin-like peptides (ELPs) and peptide active therapeutic agents.

The therapeutic compositions of the invention enable increased efficacy of the peptide active therapeutic agent, e.g., improved solubility, bioavailability, bio-unavailability (where desired to avoid build up and/or toxicity, for example cardiotoxicity, etc.), half-life of the administered peptide active therapeutic agent, etc., to be achieved, as compared to corresponding compositions containing the same peptide active therapeutic agents without associated ELPs.

For ease of reference in the ensuing discussion, set out below are definitions of specific terms appearing in such discussion.

The term “protein” is used herein in a generic sense to include polypeptides of any length.

The term “peptide” as used herein is intended to be broadly construed as inclusive of polypeptides per se having molecular weights of up to about 10,000, as well as proteins having molecular weights of greater than about 10,000, wherein the molecular weights are number average molecular weights. In a specific aspect, peptides having from about 2 to about 100 amino acid residues are particularly preferred as peptide therapeutic active agents of the invention.

As used herein, the term “coupled” means that the specified moieties are either directly covalently bonded to one another, or indirectly covalently joined to one another through an intervening moiety or moieties, such as a bridge, spacer, or linkage moiety or moieties, or they are non-covalently coupled to one another, e.g., by hydrogen bonding, ionic bonding, Van der Waals forces, etc.

As used herein, the term “half-life” means the period of time that is required for a 50% diminution of bioactivity of the active agent to occur. Such term is to be contrasted with “persistence,” which is the overall temporal duration of the active agent in the body, and “rate of clearance” as being a dynamically changing variable that may or may not be correlative with the numerical values of half-life and persistence.

The word “transform” is broadly used herein to refer to introduction of an exogenous polynucleotide sequence into a prokaryotic or eukaryotic cell by any means known in the art (including, for example, direct transmission of a polynucleotide sequence from a cell or virus particle as well as transmission by infective virus particles), resulting in a permanent or temporary alteration of genotype in an immortal or non-immortal cell line.

The term “functional equivalent” is used herein to refer to a protein that is an active analog, derivative, fragment, truncation isoform or the like of a native protein. A polypeptide is active when it retains some or all of the biological activity of the corresponding native polypeptide.

As used herein, “pharmaceutically acceptable” component (such as a salt, carrier, excipient or diluent) of a formulation according to the present invention is a component which (1) is compatible with the other ingredients of the formulation in that it can be combined with the FPs of the present invention without eliminating the biological activity of the FPs; and (2) is suitable for use with animals (including humans) without undue adverse side effects (such as toxicity, irritation, and allergic response). Side effects are “undue” when their risk outweighs the benefit provided by the pharmaceutical composition. Examples of pharmaceutically acceptable components include, without limitation, any of the standard pharmaceutical carriers such as phosphate buffered saline solutions, water, emulsions such as oil/water emulsions, microemulsions and various types of wetting agents.

As used herein, the term “native” used in reference to a protein indicates that the protein has the amino acid sequence of the corresponding protein as found in nature.

As used herein, the term “spacer” refers to any moiety that may be interposed between the ELP and the peptide active therapeutic agent in a given ELP/peptide active therapeutic agent construct. For example, the spacer may be a divalent group that is covalently bonded at one terminus to the ELP, and covalently bonded at the other terminus to the peptide active therapeutic agent. The ELP/peptide active therapeutic agent construct therefore is open to the inclusion of any additional chemical structure that does not preclude the efficacy of the ELP/peptide active therapeutic agent construct for its intended purpose. The spacer may for example be a protease-sensitive spacer moiety that is provided to control the pharmacokinetics of the ELP/peptide active therapeutic agent construct, or it may be a protease-insensitive ELP/peptide active therapeutic agent construct.

Fusion protein (FP) therapeutic compositions of the invention at least one elastin-like peptide (ELP) coupled with at least one peptide active therapeutic agent. The ELP and peptide active therapeutic agent components of the composition may be coupled with one another in any suitable manner, including covalent bonding, ionic bonding, associative bonding, complexation, or any other coupling modality that is effective to aggregate the ELP and peptide active therapeutic agent components, so that the peptide active therapeutic agent is efficacious for its intended purpose, and so that the presence of the coupled ELP enhances the peptide active therapeutic agent in the composition in some functional, therapeutic or physiological aspect, so that it is more efficacious than the peptide active therapeutic agent alone.

Thus, the ELP-coupled peptide active therapeutic agent in the FP therapeutic composition may be enhanced in any other properties, e.g., its bioavailability, bio-unavailability, therapeutic dose, formulation compatibility, resistance to proteolysis or other degradative modalities, solubility, half-life or other measure of persistence in the body subsequent to administration, rate of clearance from the body subsequent to administration, etc.

In the FP therapeutic compositions of the invention, at least one peptide active therapeutic agent is coupled to one or more ELPs, e.g., being covalently bonded at an N- or C-terminus thereof, to achieve enhancement of the efficacy of the peptide active therapeutic agent(s), in relation to the corresponding therapeutic agent(s) alone.

The FP therapeutic compositions of the invention may be therapeutically administered directly, or otherwise be produced in vivo from corresponding fusion gene constructs, including heterologous nucleotide sequences operably linked to an expression control element, e.g., a promoter of appropriate type, wherein the heterologous nucleotide sequences encode a fusion protein including at least one peptide active therapeutic agent coupled to at least one ELP.

The invention enables the enhancement of the efficacy of a peptide active therapeutic agent, e.g., by coupling the peptide active therapeutic agent with at least one ELP to form a FP therapeutic composition, wherein the peptide active therapeutic agent in such FP therapeutic composition has enhanced efficacy in relation to the peptide active therapeutic agent alone.

The invention may be practiced using any suitable therapeutic dose form including at least one peptide active therapeutic agent, coupled with at least one ELP.

The invention enables stabilization of a peptide active therapeutic agent against proteolytic degradation, by coupling such agent with at least one ELP to form a FP therapeutic composition.

The FP therapeutic composition of the invention may include one or more ELP species, and one or more peptide active therapeutic agents. As indicated hereinabove, the ELP species and peptide active therapeutic agents may be coupled directly with one another, or alternatively such coupling may be effected in a construct including a spacer moiety intermediate the ELP and the peptide active therapeutic agent.

The ELP species used in the FP therapeutic composition of the invention may be of any suitable type. ELPs are repeating peptide sequences that have been found to exist in the elastin protein. Among these repeating peptide sequences are polytetra-, polypenta-, polyhexa-, polyhepta-, polyocta, and polynonapeptides.

ELPs undergo a reversible inverse temperature transition. They are structurally disordered and highly soluble in water below a transition temperature (Tₜ), but exhibit a sharp (2-3° C. range) disorder-to-order phase transition when the temperature is raised above Tₜ, leading to desolvation and aggregation of the polypeptides. The ELP aggregates, when reaching sufficient size, can be readily removed and isolated from solution by centrifugation. Such phase transition is reversible, and isolated ELP aggregates can be completely resolubilized in buffer solution when the temperature is returned below the Tₜ of the ELPs.

In the practice of the present invention, the ELPs species functions to stabilize or otherwise improve the peptide active therapeutic agent in the therapeutic composition. Subsequent to administration of the coupled peptide active therapeutic agent-ELP construct to the patient in need of the peptide therapeutic agent, the peptide active therapeutic agent and the ELP remain coupled with one another while the peptide active therapeutic agent is therapeutically active, e.g., for treatment or prophylaxis of a disease state or physiological condition, or other therapeutic intervention.

For example, the ELPs in therapeutic compositions of the present invention may comprise ELPs formed of polymeric or oligomeric repeats of various characteristic tetra-, penta-, hexa-, hepta-, octa-, and nonapeptides, including but not limited to:

(SEQ ID NO: 1)
(a) tetrapeptide Val-Pro-Gly-Gly, or VPGG;
(SEQ ID NO: 2)
(b) tetrapeptide Ile-Pro-Gly-Gly, or IPGG;
(SEQ ID NO: 3)
(c) pentapeptide Val-Pro-Gly-X-Gly,
or VPGXG, wherein X is any natural or non-
natural amino acid residue, and wherein X
optionally varies among polymeric or oligomeric
repeats;
(SEQ ID NO: 4)
(d) pentapeptide Ala-Val-Gly-Val-Pro, or AVGVP;
(SEQ ID NO: 5)
(e) pentapeptide Ile-Pro-Gly-Val-Gly, or IPGVG;
(SEQ ID NO: 6)
(f) pentapeptide Leu-Pro-Gly-Val-Gly, or LPGVG;
(SEQ ID NO: 7)
(g) hexapeptide Val-Ala-Pro-Gly-Val-Gly, or
VAPGVG;
(SEQ ID NO: 8)
(h) octapeptide Gly-Val-Gly-Val-Pro-Gly-Val-Gly,
or GVGVPGVG;
(SEQ ID NO: 9)
(i) nonapeptide Val-Pro-Gly-Phe-Gly-Val-Gly-Ala-
Gly, or VPGFGVGAG;
and
(SEQ ID NO: 10)
(j) nonapeptides Val-Pro-Gly-Val-Gly-Val-Pro-Gly-
Gly, or VPGVGVPGG.

Any other polymeric or oligomeric repeat units of other sizes and constitutions can be usefully employed in the broad practice of the present invention.

In one embodiment, the ELP in the peptide active therapeutic agent-ELP construct includes repeat units of the pentapeptide Val-Pro-Gly-X-Gly, wherein X is as defined above, and wherein the ratio of Val-Pro-Gly-X-Gly pentapeptide units to other amino acid residues of the ELP is greater than about 75%, more preferably greater than about 85%, still more preferably greater than about 95%.

The peptide active therapeutic agent-ELP constructs of the invention may be synthetically, e.g., recombinantly, produced.

In the peptide active therapeutic agent-ELP construct, the ELP may be joined at a C- and/or N-terminus of the peptide active therapeutic agent, and optionally, a spacer sequence may be present separating the ELP from the peptide active therapeutic agent.

In one aspect, the invention contemplates a polynucleotide comprising a nucleotide sequence encoding a peptide active therapeutic agent-ELP fusion protein, optionally including a spacer sequence as above described, separating the ELP from the peptide active therapeutic agent. The polynucleotide may be provided as a component of an expression vector. The invention also contemplates a host cell (prokaryotic or eukaryotic) transformed by such expression vector to express the fusion protein.

The peptide active therapeutic agent-ELP construct subsequent to its synthesis or expression can be isolated by a method involving effecting a phase transition, e.g., by raising temperature, or in other manner, producing a phase transition of the fusion protein in the medium in which is contained in non-isolated form.

For example, the peptide active therapeutic agent-ELP construct may be synthesized and recovered, by steps including forming a polynucleotide comprising a nucleotide sequence encoding a peptide active therapeutic agent-ELP fusion protein exhibiting a phase transition, expressing the fusion protein in culture, and subjecting fusion protein-containing material from the culture to processing involving separation (e.g., by centrifugation, membrane separation, etc.) and inverse transition cycling to recover the peptide active therapeutic agent-ELP fusion protein.

In one specific embodiment, the peptide active therapeutic agent-ELP fusion protein includes an ELP moiety including polymeric or oligomeric repeats of a polypeptide selected from the group consisting of VPGG, IPGG, AVGVP, IPGVG, LPGVG, VAPGVG, GVGVPGVG, VPGFGVGAG, and VPGVGVPGG.

In another specific embodiment, the peptide active therapeutic agent-ELP fusion protein includes an ELP moiety including polymeric or oligomeric repeat units selected from the group consisting of LPGXG (SEQ ID NO: 11), IPGXG (SEQ ID NO: 12), and combinations thereof, wherein X is an amino acid residue that does not preclude phase transition of the ELP fusion protein.

The peptide active therapeutic agent-ELP construct of the invention comprises an amino acid sequence endowing the construct with phase transition characteristics.

The ELP in the peptide active therapeutic agent-ELP construct can include β-turn component. Examples of polypeptides suitable for use as the β-turn component are described in Urry, et al. International Patent Application PCT/US96/05186. Alternatively, the ELP in the peptide active therapeutic agent-ELP construct can be a component lacking a β-turn component, or otherwise having a different conformation and/or folding character.

The ELPs, as mentioned, can include polymeric or oligomeric repeats of various tetra-, penta-, hexa-, hepta-, octa-, and nonapeptides, including but not limited to VPGG, IPGG, VPGXG, AVGVP, IPGVG, LPGVG, VAPGVG, GVGVPGVG, VPGFGVGAG, and VPGVGVPGG (SEQ NO: 1 to SEQ NO: 10). It will be appreciated by those of skill in the art that the ELPs need not consist of only polymeric or oligomeric sequences as listed hereinabove, in order to exhibit a phase transition or otherwise constitute a suitable ELPs species for use in the peptide active therapeutic agent-ELP constructs of the invention.

In one embodiment, the peptide active therapeutic agent-ELP construct includes ELPs that are polymeric or oligomeric repeats of the pentapeptide VPGXG (SEQ ID NO: 3), where the guest residue X is any amino acid that does not eliminate the phase transition characteristics of the ELP. X may be a naturally occurring or non-naturally occurring amino acid. For example, X may be selected from the group consisting of: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine. In a specific embodiment, X is not proline.

X may be a non-classical amino acid. Examples of non-classical amino acids include: D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

Selection of the identity of X is independent in each ELP repetition. Selection may be based on any desired characteristic, such as consideration of positively charged or negatively charged residues in the X position. It may be considered that ELPs with neutral values in the X position may have longer half-lives.

In another embodiment, the peptide active therapeutic agent-ELP construct includes ELPs that are polymeric or oligomeric repeats of the pentapeptide IPGXG (SEQ ID NO: 11) or LPGXG (SEQ ID NO: 12), where X is as defined hereinabove.

The polymeric or oligomeric repeats of the ELP sequences may be separated by one or more amino acid residues that do not eliminate the overall phase transition characteristic of the peptide active therapeutic agent-ELP construct. In one specific embodiment, when the ELP component of the peptide active therapeutic agent-ELP construct comprises polymeric or oligomeric repeats of the pentapeptide VPGXG, the ratio of VPGXG repeats to other amino acid residues of the ELP is greater than about 75%, more preferably greater than about 85%, still more preferably greater than about 95%, and most preferably greater than about 99%.

In each repeat, X is independently selected. Different resulting ELP species are distinguished here using the notation ELPk [XᵢYⱼ-n], where k designates the specific type of ELP repeat unit, the bracketed capital letters are single letter amino acid codes and their corresponding subscripts designate the relative ratio of each guest residue X in the repeat units, and n describes the total length of the ELP in number of the pentapeptide repeats. For example, ELP1 [V₅A₂G₃-10] designates a polypeptide containing 10 repeating units of the pentapeptide VPGXG, where X is valine, alanine, and glycine at a relative ratio of 5:2:3; ELP1 [K₁V₂F₁-4] designates a polypeptide containing 4 repeating units of the pentapeptide VPGXG, where X is lysine, valine, and phenylalanine at a relative ratio of 1:2:1; ELP1 [K₁V₇F₁-9] designates a polypeptide containing 4 repeating units of the pentapeptide VPGXG, where X is lysine, valine, and phenylalanine at a relative ratio of 1:7:1; ELP1 [V-5] designates a polypeptide containing 5 repeating units of the pentapeptide VPGXG, where X is exclusively valine; ELP1 [V-20] designates a polypeptide containing 20 repeating units of the pentapeptide VPGXG, where X is exclusively valine; ELP2 [5] designates a polypeptide containing 5 repeating units of the pentapeptide AVGVP; ELP3 [V-5] designates a polypeptide containing 5 repeating units of the pentapeptide IPGXG, where X is exclusively valine; ELP4 [V-5] designates a polypeptide containing 5 repeating units of the pentapeptide LPGXG, where X is exclusively valine.

Previous studies by Urry and colleagues have shown that the fourth residue (X) in the elastin pentapeptide sequence, VPGXG, can be altered without eliminating the formation of the β-turn. These studies also showed that the Tₜ is a function of the hydrophobicity of the guest residue. By varying the identity of the guest residue(s) and their mole fraction(s), ELPs can be synthesized that exhibit an inverse transition over a 0-100° C. range.

The Tₜ at a given ELP length can be decreased by incorporating a larger fraction of hydrophobic guest residues in the ELP sequence. Examples of suitable hydrophobic guest residues include valine, leucine, isoleucine, phenyalanine, tryptophan and methionine. Tyrosine, which is moderately hydrophobic, may also be used. Conversely, the Tₜ can be increased by incorporating residues, such as those selected from the group consisting of: glutamic acid, cysteine, lysine, aspartate, alanine, asparagine, serine, threonine, glysine, arginine, and glutamine; preferably selected from alanine, serine, threonine and glutamic acid.

The ELP in one embodiment is selected to provide a Tₜ ranging from about 10 to about 80° C., more preferably from about 35 to about 60° C., most preferably from about 38 to about 45° C.

The Tₜ can also be varied by varying ELP chain length. The Tₜ increases with decreasing MW. For polypeptides having a molecular weight >100,000, the hydrophobicity scale developed by Urry et al. (PCT/US96/05186) is preferred for predicting the approximate Tₜ of a specific ELP sequence.

For polypeptides having a molecular weight <100,000, the Tₜ is preferably determined by the following quadratic function:

Tₜ=M₀+M₁X+M₂X²

where X is the MW of the FP, and M₀=116.21; M₁=−1.7499; M₂=0.010349.

While the Tₜ of the ELP and, therefore of a construct of an ELP linked to a peptide active therapeutic agent, is affected by the identity and hydrophobicity of the guest residue, X, additional properties of the construct may also be affected. Such properties include, but are not limited to solubility, bioavailability or bio-unavailability, and half-life of the ELP itself and the construct.

In the Examples section below, it is seen that the ELP-coupled active therapeutic agent retains a significant amount of the therapeutic agent's biological activity, as compared to free protein forms of such therapeutic agent. Additionally, it is shown that ELPs exhibit long half-lives. Correspondingly, ELPs can be used in accordance with the invention to substantially increase (e.g. by greater than 10%, 20%, 30%, 50%, 100%, 200% or more, in specific embodiments) the half-life of the therapeutic agent, as conjugated with an ELP, in comparison to the half-life of the free (unconjugated) form of the therapeutic agent. Furthermore, ELPs are shown to target high blood content organs, when administered in vivo, and thus, can partition in the body, to provide a predetermined desired corporeal distribution among various organs or regions of the body, or a desired selectivity or targeting of a therapeutic agent. In sum, active ELP-therapeutic agent conjugates contemplated by the invention are administered or generated in vivo as active, site-specific compositions having extended half-lives.

In one embodiment of the invention, the ELP length is from 5 to about 500 amino acid residues, more preferably from about 10 to about 450 amino acid residues, and still more preferably from about 15 to about 150 amino acid residues. ELP length can be reduced while maintaining a target Tₜ by incorporating a larger fraction of hydrophobic guest residues in the ELP sequence.

The active therapeutic agent in the peptide active therapeutic agent-ELP construct can be of any suitable type. Suitable peptides include those of interest in medicine, agriculture and other scientific and industrial fields, particularly including therapeutic proteins such as erythropoietins, magainins, beta-defensins, inteferons, insulin, monoclonal antibodies, blood factors, colony stimulating factors, growth hormones, interleukins, growth factors, therapeutic vaccines, calcitonins, tumor necrosis factors (TNF), receptor antagonists, corticosteroids, and enzymes. Specific examples of such peptides include, without limitation, enzymes utilized in replacement therapy; antibacterial peptides; hormones for promoting growth; and active proteinaceous substances used in various applications. Specific examples include, but are not limited to: superoxide dismutase, interferon, asparaginease, glutamase, arginase, arginine deaminase, adenosine deaminase ribonuclease, trypsin, chromotrypsin, papin, insulin, calcitonin, ACTH, glucagon, glucagon-like peptide-1 (GLP-1), somatosin, somatropin, somatomedin, parathyroid hormone, erthyropoietin, hypothalamic releasing factors, prolactin, thyroid stimulating hormones, endorphins, enkephalins, and vasopressin.

In one embodiment of the invention, the peptide active therapeutic agent is thioredoxin.

In another embodiment, the peptide active therapeutic agent is tendamistat. The tendamistat-ELP fusion protein provides a readily-isolated, active version of tendamistat for use as an α-amylase inhibitor, e.g., in the treatment of pancreatitis. This fusion protein is suitably provided as a component of a pharmaceutical formulation in association with a pharmaceutically acceptable carrier. The tendamistat-ELP fusion protein retains most of the α-amylase inhibition activity of the free tendamistat, and is a stable construct.

In one specific embodiment, the peptide active therapeutic agent includes a physiologically active peptide selected from the group consisting of insulin, calcitonin, ACTH, glucagon, somatostatin, somatotropin, somatomedin, parathyroid hormone, erythropoietin, hypothalmic releasing factors, prolactin, thyroid stimulating hormones, endorphins, enkephalins, vasopressin, non-naturally occurring opiods, superoxide dismutase, interferon, asparaginase, arginase, arginine deaminease, adenosine deaminase ribonuclease, trypsin, chymotrypsin, and papain.

The invention thus comprehends various compositions for therapeutic (in vivo) application, wherein the peptide component of the peptide active therapeutic agent-ELP construct is a physiologically active, or bioactive, peptide. In preferred forms of such peptide-containing compositions, the coupling of the peptide component to ELP species is effected by direct covalent bonding or indirect (through appropriate spacer groups) bonding, and the peptide and ELP moieties can be structurally arranged in any suitable manner involving such direct or indirect covalent bonding, relative to one another. Thus, a wide variety of peptide species can be accommodated in the broad practice of the present invention, as necessary or desirable in a given therapeutic application.

The peptides utilized as peptide active therapeutic agents in the peptide active therapeutic agent-ELP constructs of the invention in one embodiment include enzymes utilized in replacement therapy and hormones for promoting growth. Among such enzymes are superoxide dismutase, interferon, asparaginease, glutamase, arginase, arginine deaminase, adenosine deaminase ribonuclease, cytosine deaminase, trypsin, chromotrypsin, and papin. Among the peptide hormones, specific species amenable to use in the peptide active therapeutic agent-ELP constructs of the invention include, without limitation, insulin, calcitonin, ACTH, glucagon, somatosin, somatropin, somatomedin, parathyroid hormone, erthyropoietin, hypothalamic releasing factors, prolactin, thyroid stimulating hormones, endorphins, enkephalins, and vasopressin.

In another specific aspect, the peptide active therapeutic agent in the ELPs/peptide active therapeutic agent construct is selected from among the following species, and all variants, fragments and derivatives of such species: agouti related peptide, amylin, angiotensin, cecropin, bombesin, gastrin, including gastrin releasing peptide, lactoferin, antimicrobial peptides including but not limited to magainin, urodilatin, nuclear localization signal (NLS), collagen peptide, survivin, amyloid peptides, including β-amyloid, natiuretic peptides, peptide YY, neuroregenerative peptides and neuropeptides, including but not limited to neuropeptide Y, dynorphin, endomorphin, endothelin, enkaphalin, exendin, fibronectin, neuropeptide W and neuropeptide S, peptide T, melanocortin, amyloid precursor protein, sheet breaker peptide, CART peptide, amyloid inhibitory peptide, prion inhibitory peptide, chlorotoxin, corticotropin releasing factor, oxytocin, vasopressin, cholecystokinin, secretin, thymosin, epidermal growth factor (EGF), vascular endothelial cell growth factor (VEGF), platelet-derived growth factor (PDGF), Insulin-like growth factor (IGF), fibroblast growth factors (aFGF, bFGF), pancreastatin, melanocyte stimulating hormone, osteocalcin, bradykinin, adrenomedullin, perinerin, metastatin, aprotinin, galanins, including galanin-like peptide, leptin, defensins, including but not limited to α-defensin and β-defensin, salusin, and various venoms, including but not limited to conotoxin, decorsin, kurtoxin, anenomae venom, tarantula venom; natriuretic peptides including brain natriuretic peptide (B-type natriuretic peptide, or BNP), atrial natriuretic peptide, and vasonatrin; neurokinin A, neurokinin B; neuromedin; neurotensin; orexin, pancreatic polypeptide, pituitary adenylate cyclase activating peptide (PACAP), prolactin releasing peptide, proteolipid protein (PLP), somatostatin, TNF-α; Grehlin, Protein C (Xigris), SS1(dsFv)-PE38 and pseudomonas exotoxin protein, clotting factors, including antithrombin III and Coagulation Factor VIIA, Factor VIII, Factor IX, streptokinase, tissue plasminogen activators, urokinase, beta glucocerebrosidase and alpha-D-galactosidase, alpha L-iduronidase, alpha-1,4-glucosidase, arylsulfatase B, iduronate-2-sulfatase, deoxyribunuclase I, human activated protein, follicle-stimulating hormone, chorionic gonadotropin, luteinizing hormone, somatropin, bone morphogenetic protein, nesiritide, parathyroid hormone, erythropoietin, keratinocyte growth factor, human granulocyte colony-stimulating factor (G-CSF), human granulocyte-macrophase colony stimulating factor (GM-CSF), alpha interferon, beta interferon, gamma interferon, interleukins, including IL-1, IL-1Ra, IL-2, Il-4, IL-5, IL-6, IL-10, IL-11, IL-12, glycoprotein IIB/IIIA, immune globulins, including hepatitis B, gamma globulin, venoglobulin, hirudin, aprotinin, antithrombin III, alpha-1-proteinase inhibitor, filgrastim, and etanercept.

In another embodiment, the peptide component of the peptide active therapeutic agent-ELP constructs of the present invention may be an antibody or antigen, in connection with immunotherapy, or other therapeutic intervention.

Various other proteins and peptides, such as insulin A peptide, T20 peptide, interferon alpha 2B peptide, tobacco etch virus protease, small heterodimer partner orphan receptor, androgen receptor ligand binding domain, glucocorticoid receptor ligand binding domain, estrogen receptor ligand binding domain, G protein alpha Q, 1-deoxy-D-xylulose 5-phosphate reductoisomerase peptide, G protein alpha S, angiostatin (K1-3), blue fluorescent protein (BFP), calmodulin (CalM), chloramphenicol acetyltransferase (CAT), green fluorescent protein (GFP), interleukin 1 receptor antagonist (IL-1Ra), luciferase, tissue transglutaminase (tTg), morphine modulating neuropeptide (MMN), neuropeptide Y (NPY), orexin-B, leptin, ACTH, calcitonin, adrenomedullin (AM), parathyroid hormone (PTH), defensin and growth hormone have been fused with different ELP polypeptides to form FPs that exhibit inverse phase transition behavior.

The proteins and peptides employed as active therapeutic agents can be significantly different in their primary, secondary, and tertiary structures, sizes, molecular weights, solubility, electric charge distribution, viscosity, and biological functions.

Also included within the scope of the invention are derivatives comprising FPs, which have been differentially modified during or after synthesis, e.g., by benzylation, glycosylation, acetylation, phosphorylation, amidation, PEGylation, derivatization by known protecting/blocking groups, proteolytic cleavage, linkage to an antibody molecule or other cellular ligand, etc. In one embodiment, the FPs are acetylated at the N-terminus and/or amidated at the C-terminus. In another embodiment, the FPs are conjugated to polymers, e.g., polymers known in the art to facilitate oral delivery, decrease enzymatic degradation, increase solubility of the polypeptides, or otherwise improve the chemical properties of the therapeutic polypeptides for administration to humans or other animals.

The peptide active therapeutic agent-ELP constructs of the invention can be obtained by known recombinant expression techniques. To recombinantly produce the peptide active therapeutic agent-ELP construct, a nucleic acid sequence encoding the construct is operatively linked to a suitable promoter sequence such that the nucleic acid sequence encoding such fusion peptide will be transcribed and/or translated into the desired fusion peptide in the host cells. Preferred promoters are those useful for expression in E. coli, such as the T7 promoter.

Any commonly used expression system may be used, e.g., eukaryotic or prokaryotic systems. Specific examples include yeast, pichia, baculovirus, mammalian, and bacterial systems, such as E. coli, and Caulobacter.

A vector comprising the nucleic acid sequence can be introduced into a cell for expression of the peptide active therapeutic agent-ELP construct. The vector can remain episomal or become chromosomally integrated, as long as the gene carried by it can be transcribed to produce the desired RNA. Vectors can be constructed by standard recombinant DNA technology methods. Vectors can be plasmids, phages, cosmids, phagemids, viruses, or any other types known in the art, which are used for replication and expression in prokaryotic or eukaryotic cells. It will be appreciated by one of skill in the art that a wide variety of components known in the art may be included in such vectors, including a wide variety of transcription signals, such as promoters and other sequences that regulate the binding of RNA polymerase onto the promoter. Any promoter known to be effective in the cells in which the vector will be expressed can be used to initiate expression of the peptide active therapeutic agent-ELP construct. Suitable promoters may be inducible or constitutive. Examples of suitable promoters include the SV40 early promoter region, the promoter contained in the 3′ long terminal repeat of Rous sarcoma virus, the HSV-1 (herpes simplex virus-1) thymidine kinase promoter, the regulatory sequences of the metallothionein gene, etc., as well as the following animal transcriptional control regions, which exhibit tissue specificity and have been utilized in transgenic animals: elastase I gene control region which is active in pancreatic acinar cells; insulin gene control region which is active in pancreatic beta cells, immunoglobulin gene control region which is active in lymphoid cells, mouse mammary tumor virus control region which is active in testicular, breast, lymphoid and mast cells, albumin gene control region which is active in liver, alpha-fetoprotein gene control region which is active in liver, alpha 1-antitrypsin gene control region which is active in the liver, beta-globin gene control region which is active in erythroid cells, myelin basic protein gene control region which is active in oligodendrocyte cells in the brain, myosin light chain-2 gene control region which is active in skeletal muscle, and gonadotropin releasing hormone gene control region which is active in the hypothalamus.

In one embodiment, a mammal is genetically modified to produce the peptide active therapeutic agent-ELP construct in its milk. Techniques for performing such genetic modifications are described in U.S. Pat. No. 6,013,857, issued Jan. 11, 2000, for “Transgenic Bovines and Milk from Transgenic Bovines.” The genome of the transgenic animal is modified to comprise a transgene comprising a DNA sequence encoding a peptide active therapeutic agent-ELP construct operably linked to a mammary gland promoter. Expression of the DNA sequence results in the production of the peptide active therapeutic agent-ELP construct in the milk. The peptide active therapeutic agent-ELP construct can then be isolated by phase transition from milk obtained from the transgenic mammal. The transgenic mammal is preferably a bovine.

The peptide active therapeutic agent-ELP constructs of the invention can be separated from other contaminating proteins to high purity using inverse transition cycling procedures, e.g., utilizing the temperature-dependent solubility of the peptide active therapeutic agent-ELP construct, or salt addition to the medium containing the construct. Successive inverse phase transition cycles can be used to obtain a high degree of purity.

In addition to temperature and ionic strength, other environmental variables useful for modulating the inverse transition of peptide active therapeutic agent-ELP constructs include pH, the addition of inorganic and organic solutes and solvents, side-chain ionization or chemical modification, and pressure.

In one specific illustrative embodiment of the invention, a 10 polypentapeptide ELP (an ELP 10-mer) is constructed. The ELP 10-mer may be oligomerized or polymerized up to 18 times to create a library of ELPs with precisely specified molecular masses (10-, 20-, 30-, 60-, 90-, 120-, 150-, and 180-mers). The ELP polymers or oligomers may then be fused to the C- or N-terminus of the peptide active therapeutic agent, to form the peptide active therapeutic agent-ELP construct. A second peptide active therapeutic agent may be fused to the ELP component of the fusion protein construct, providing a ternary fusion. Optionally, one or more spacers may be used to separate the ELP component from the peptide active therapeutic agent(s).

The invention thus affords a peptide active therapeutic agent-ELP construct in which the peptide active therapeutic agent may be a natural or synthetic version of any of a wide variety of endogenous molecules, or alternatively a non-naturally-occurring peptide species, or a functional equivalent of any of the foregoing.

The peptide active therapeutic agent-ELP constructs of the invention overcome the major deficiency of peptide active therapeutic agents when given parenterally, namely, that such peptides are easily metabolized by plasma proteases. The oral route of administration of peptide active therapeutic agents is even more problematic because in addition to proteolysis in the stomach, the high acidity of the stomach destroys such peptide active therapeutic agents before they reach their intended target tissue. Peptides and peptide fragments produced by the action of gastric and pancreatic enzymes are cleaved by exo and endopeptidases in the intestinal brush border membrane to yield di- and tripeptides, and even if proteolysis by pancreatic enzymes is avoided, polypeptides are subject to degradation by brush border peptidases. Any of the peptide active therapeutic agents that survive passage through the stomach are further subjected to metabolism in the intestinal mucosa where a penetration barrier prevents entry into the cells. The peptide active therapeutic agent-ELP constructs of the invention overcome such deficiencies, and provide compositional forms of the peptide active therapeutic agent having enhanced efficacy, in bioavailability, bio-unavailability, therapeutic half-life, degradation assistance, etc.

The peptide active therapeutic agent-ELP constructs of the invention thus enable oral and parenteral dose forms, as well as various other dose forms, by which peptide active therapeutic agents can be utilized in a highly effective manner. For example, such constructs enable dose forms that achieve high mucosal absorption, and the concomitant ability to use lower doses to elicit an optimum therapeutic effect.

The ELP/peptide active therapeutic agent construct may also include a spacer as a moiety in the construct. The spacer may be of any suitable type, and may be a peptide spacer, or alternatively a non-peptide chemical moiety.

Peptide spacers may be protease-cleavable or non-cleavable. By way of example, cleavable peptide spacer species include, without limitation, in a peptide sequences recognized by proteases of varying type, such as thrombin, factor Xa, plasmin (blood proteases), metalloproteases, cathepsins (e.g., GFLG, etc.), and proteases found in other corporeal compartments. The non-cleavable spacer may likewise be of any suitable type, including, for example, non-cleavable spacer moieties having the formula [(Gly)ₙ-Ser]ₘ where n is from 1 to 4, inclusive, and m is from 1 to 4, inclusive.

Non-peptide chemical spacers can additionally be of any suitable type, including for example, by functional linkers described in Bioconjugate Techniques, Greg T. Hermanson, published by Academic Press, Inc., 1995, and those specified in the Cross-Linking Reagents Technical Handbook, available from Pierce Biotechnology, Inc. (Rockford, Ill.), the disclosures of which are hereby incorporated by reference, in their respective entireties. Illustrative chemical spacers include homobifunctional linkers that can attach to amine groups of Lys, as well as heterobifunctional linkers that can attach to Cys at one terminus, and to Lys at the other terminus, and other bifunctional linkers that can link proteins to the Fc region of antibodies, in which the antibody's carbohydrate is first oxidized to a diol or aldehyde.

The peptide active therapeutic agent-ELP constructs of the invention have application in prophylaxis or treatment of condition(s) or disease state(s). Although such constructs are described herein with reference to peptide active therapeutic agents having utility for animal subjects, the invention also contemplates peptide active therapeutic agent-ELP constructs having utility for prophylaxis or treatment of condition(s) or disease state(s) in plant systems. By way of example, the peptide component of the peptide active therapeutic agent-ELP construct having such plant utility may have insecticidal, herbicidal, fungicidal, and/or pesticidal efficacy.

A further aspect of the invention relates to gene therapy utilizing fusion gene therapeutic compositions of the invention, in conjunction with vectors of any suitable type, e.g., AAV, vaccinia, pox virus, HSV, retrovirus, lipofection, RNA transfer, etc.

In therapeutic usage, the present invention contemplates a method of treating an animal subject having or latently susceptible to such condition(s) or disease state(s) and in need of such treatment, including administering to such animal an effective amount of a peptide active therapeutic agent-ELP construct of the present invention which is therapeutically effective for said condition or disease state.

Animal subjects to be treated by the peptide active therapeutic agent-ELP constructs of the present invention include both human and non-human animal (e.g., bird, dog, cat, cow, horse) subjects, and preferably are mammalian subjects, and most preferably human subjects.

Depending on the specific condition or disease state to be combated, animal subjects may be administered peptide active therapeutic agent-ELP constructs of the invention at any suitable therapeutically effective and safe dosage, as may readily be determined within the skill of the art, without undue experimentation, based on the disclosure herein.

In general, suitable doses of the peptide active therapeutic agent in the peptide active therapeutic agent-ELP construct for achievement of therapeutic benefit, can for example be in a range of 1 microgram (μg) to 100 milligrams (mg) per kilogram body weight of the recipient per day, preferably in a range of 10 μg to 50 mg per kilogram body weight per day and most preferably in a range of 10 μg to 50 mg per kilogram body weight per day. The desired dose can be presented as two, three, four, five, six, or more sub-doses administered at appropriate intervals throughout the day. These sub-doses can be administered in unit dosage forms, for example, containing from 10 μg to 1000 mg, preferably from 50 μg to 500 mg, and most preferably from 50 μg to 250 mg of active ingredient per unit dosage form. Alternatively, if the condition of the recipient so requires, the doses may be administered as a continuous infusion.

The mode of administration and dosage forms will of course affect the therapeutic amount of the peptide active therapeutic agent that is desirable and efficacious for a given treatment application.

For example, orally administered dosages can be at least twice, e.g., 2-10 times, the dosage levels used in parenteral administration methods, for the same peptide active therapeutic agent.

The peptide active therapeutic agent-ELP constructs of the invention may be administered per se as well as in forms of such constructs including pharmaceutically acceptable esters, salts, and other physiologically functional derivatives thereof.

The present invention also contemplates pharmaceutical formulations, both for veterinary and for human medical use, which include peptide active therapeutic agent-ELP constructs of the invention.

In such pharmaceutical and medicament formulations, the peptide active therapeutic agent-ELP construct can be utilized together with one or more pharmaceutically acceptable carrier(s) therefore and optionally any other therapeutic ingredients. The carrier(s) must be pharmaceutically acceptable in the sense of being compatible with the other ingredients of the formulation and not unduly deleterious to the recipient thereof. The peptide active therapeutic agent-ELP construct is provided in an amount effective to achieve the desired pharmacological effect, as described above, and in a quantity appropriate to achieve the desired daily dose.

The formulations of the peptide active therapeutic agent-ELP constructs include those suitable for parenteral as well as non-parenteral administration, and specific administration modalities include oral, rectal, buccal, topical, nasal, ophthalmic, subcutaneous, intramuscular, intravenous, transdermal, intrathecal, intra-articular, intra-arterial, sub-arachnoid, bronchial, lymphatic, vaginal, and intra-uterine administration. Formulations suitable for oral and parenteral administration are preferred.

When the peptide active therapeutic agent-ELP construct is utilized in a formulation including a liquid solution, the formulation advantageously can be administered orally or parenterally. When the peptide active therapeutic agent-ELP construct is employed in a liquid suspension formulation or as a powder in a biocompatible carrier formulation, the formulation may be advantageously administered orally, rectally, or bronchially.

When the peptide active therapeutic agent-ELP construct is utilized directly in the form of a powdered solid, the active agent can be advantageously administered orally. Alternatively, it may be administered bronchially, via nebulization of the powder in a carrier gas, to form a gaseous dispersion of the powder which is inspired by the patient from a breathing circuit comprising a suitable nebulizer device.

The formulations comprising the peptide active therapeutic agent-ELP constructs of the present invention may conveniently be presented in unit dosage forms and may be prepared by any of the methods well known in the art of pharmacy. Such methods generally include the step of bringing the peptide active therapeutic agent-ELP construct(s) into association with a carrier which constitutes one or more accessory ingredients. Typically, the formulations are prepared by uniformly and intimately bringing the peptide active therapeutic agent-ELP construct(s) into association with a liquid carrier, a finely divided solid carrier, or both, and then, if necessary, shaping the product into dosage forms of the desired formulation.

Formulations of the present invention suitable for oral administration may be presented as discrete units such as capsules, cachets, tablets, or lozenges, each containing a predetermined amount of the active ingredient as a powder or granules; or a suspension in an aqueous liquor or a non-aqueous liquid, such as a syrup, an elixir, an emulsion, or a draught.

A tablet may be made by compression or molding, optionally with one or more accessory ingredients. Compressed tablets may be prepared by compressing in a suitable machine, with the peptide active therapeutic agent-ELP construct(s) being in a free-flowing form such as a powder or granules which optionally is mixed with a binder, disintegrant, lubricant, inert diluent, surface active agent, or discharging agent. Molded tablets comprised of a mixture of the powdered peptide active therapeutic agent-ELP construct(s) with a suitable carrier may be made by molding in a suitable machine.

A syrup may be made by adding the peptide active therapeutic agent-ELP construct(s) to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

Formulations suitable for parenteral administration conveniently comprise a sterile aqueous preparation of the peptide active therapeutic agent-ELP construct(s), which preferably is isotonic with the blood of the recipient (e.g., physiological saline solution). Such formulations may include suspending agents and thickening agents or other microparticulate systems which are designed to target the peptide active therapeutic agent to blood components or one or more organs. The formulations may be presented in unit-dose or multi-dose form.

Nasal spray formulations comprise purified aqueous solutions of the peptide active therapeutic agent-ELP construct(s) with preservative agents and isotonic agents. Such formulations are preferably adjusted to a pH and isotonic state compatible with the nasal mucus membranes.

Formulations for rectal administration may be presented as a suppository with a suitable carrier such as cocoa butter, hydrogenated fats, or hydrogenated fatty carboxylic acid.

Ophthalmic formulations are prepared by a similar method to the nasal spray, except that the pH and isotonic factors are preferably adjusted to match that of the eye.

Topical formulations comprise the peptide active therapeutic agent-ELP construct(s) dissolved or suspended in one or more media, such as mineral oil, petroleum, polyhydroxy alcohols, or other bases used for topical pharmaceutical formulations.

In addition to the aforementioned ingredients, the formulations of this invention may further include one or more accessory ingredient(s) selected from diluents, buffers, flavoring agents, disintegrants, surface active agents, thickeners, lubricants, preservatives (including antioxidants), and the like.

The features and advantages of the present invention are more fully shown with respect to the following non-limiting examples.

### EXAMPLES

Features of the invention are more fully shown with illustrative reference to experiments involving the expression of fusion proteins containing various different recombinant proteins, such as thioredoxin, tendamistat, insulin, T20 protein, interferon, tobacco etch virus protease, small heterodimer partern orphan receptor, androgen receptor ligand binding protein, glucocorticoid receptor ligand binding protein, estrogen receptor ligand binding protein, G proteins, 1-deoxy-D xylulose 5-phosphate reductoisomerase, angiostatin (K1-3), blue fluorescent protein (BFP), calmodulin (CalM), chloramphenicol acetyltransferase (CAT), green fluorescent protein (GFP), interleukin 1 receptor antagonist (IL-1Ra), luciferase, tissue transglutaminase (tTg), morphine modulating neuropeptide (MMN), neuropeptide Y (NPY), orexin-B, leptin, ACTH, calcitonin, adrenomedullin (AM), parathyroid hormone (PTH), defensin and growth hormone that are fused to various different ELP sequences.

### Example 1

### Production and Purification of Proteins and Long Peptides

In the case studies presented, E. coli strain BL21 star (Invitrogen) containing ELP-(TEV)-peptide/protein constructs were grown in media supplemented with antibiotic at 37° C. for 24 hrs without induction. The culture was harvested and resuspended in 50 mM Tris-HCL pH 8.0 and 1 mM EDTA. Cells were lysed by ultrasonic disruption on ice. Cell debris was removed by centrifugation at 20,000 g at 4° C. for 30 minutes. Inverse temperature transition was induced by adding NaCl to a final concentration of 1.5 M to the lysate at 25° C., followed by centrifugation at 20,000 g for 15 minutes at 25° C. The resulting pellet contained ELP-(TEV)-peptide/protein fusion and non-specifically NaCl precipitated proteins. The pellet was resuspended in 40 ml ice-cold buffer and centrifuged at 20,000 g, 4° C. for 15 minutes to remove non-specific insoluble proteins. The temperature transition cycle was repeated three additional times to increase the purity of ELP-TEV fusion protein and to reduce the final volume to less than 5 ml.

Separation of the peptide/protein from ELP was achieved by adding ELP-TEV protease and incubating at 25° C. for 18 hrs. Cleaved peptide/protein was further purified from ELP and ELP-TEV protease using a final temperature transition in the presence of 0.5 M NaCl followed by centrifugation at 10,000 g at room temperature. NaCl transitioned ELP, ELP-TEV protease and non-cleaved ELP-peptide/protein are found in the insoluble fraction while the peptide/protein remained in the soluble fraction. HPLC and liquid chromatography mass spectrum (LC-MS) analysis was carried out to test how accurately TEV cleaved ELP-(TEV)-peptide/protein and final purity of the peptide/protein. The concentration of ELP-(TEV)-peptide/protein, ELP and purified peptide/protein was determined spectrophotometrically using extinction coefficients calculated by ExPASy tools ProtParam. (19^{th} Annual American Peptide Symposium, June 2005; poster presentation.)

### Production of a 37 Amino Acid Peptide

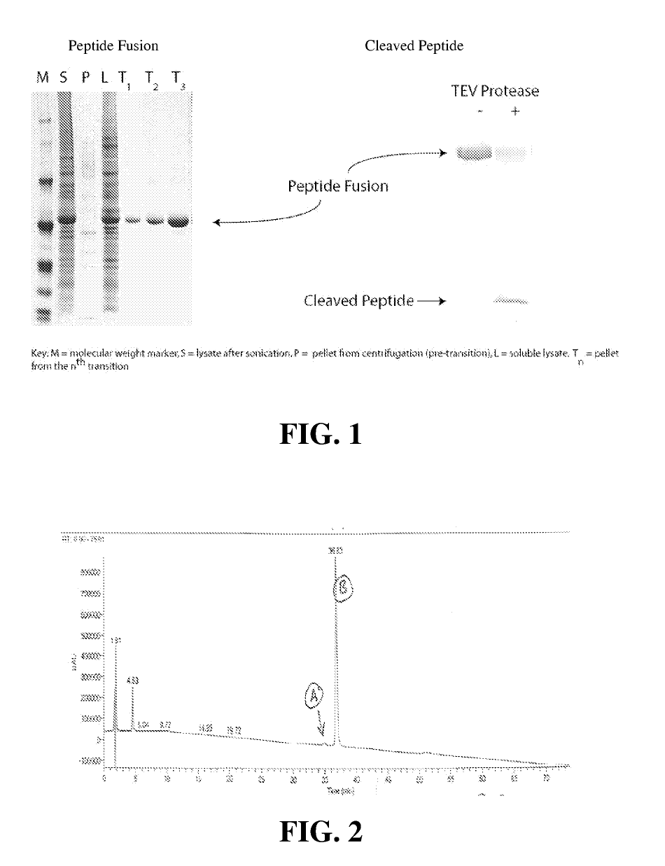

A 37 amino acid peptide was expressed and purified using the above (deltaPhase™) system. The expressed ELP-peptide fusion was purified through several rounds of transitions. The purified fusion was incubated with TEV protease to cleave the peptide. The TEV protease was prepared as an ELP fusion in a separate experiment which allowed removal from solution along with the cleaved ELP after incubation. Results are shown in FIG. 1, where M is the molecular weight marker, S is the lysate after sonication, P is the pellet from centrifugation (pre-transition), L is the soluble lysate, and Tₙ is the pellet from the n^{th} transition.

The resulting peptide had greater than 90% purity with a minor deamidated impurity, as is seen in FIG. 2, the graph results of confirmation of molecular weight and purity by LC-MS.

### Rapid Production of a Series of Peptide Variants

The throughput and purity possible for a series of peptides was then determined. The results, shown in Table 1, demonstrate the ability to produce consistent results across a series of peptides. Previously, the limits of chemical synthesis limited peptide production to one peptide every 3 to 6 weeks, which limited the rate of peptide optimization. Using the deltaPhase™ System, as set forth above, the following six peptides could be produced in less than two weeks. Given the ability to parallel process this system, the throughput could have easily been increased to several hundred in several weeks.

TABLE 1
Yield and Purity for a Series of Peptide Variants.

| | |
|---|---|
| Final Yield | Peptide |

| | ELP-Peptide | Peptide | Purity Level |
|---|---|---|---|
| Peptide | (mg/L) | (mg/L) | (LC-MS) |
| Core | 280 | 18 | 94% |
| Variant 1 | 389 | 32 | 93% |
| Variant 2 | 194 | 20 | 90% |
| Variant 3 | 195 | 21 | 98% |
| Variant 4 | 267 | 32 | 92% |
| Variant 5 | 195 | 20 | 92% |

### Example 2

### Fusion Proteins Containing Thioredoxin and/or Tendamistat

Thioredoxin and tendamistat exemplify two limiting scenarios of protein expression: (1) the peptide active therapeutic agent over-expresses at high levels and is highly soluble (thioredoxin), and (2) the peptide active therapeutic agent is expressed largely as insoluble inclusion bodies (tendamistat).

The thioredoxin-ELP fusion protein exhibited only a small increase in Tₜ (1-2° C.) compared to free ELP, while the tendamistat fusion displayed a more dramatic 15° C. reduction in Tₜ. This shift was identical for both the ternary (thioredoxin-ELP-tendamistat) and binary (ELP-tendamistat) constructs, indicating that the Tₜ shift was associated specifically with tendamistat. These observations are consistent with the conclusion that the decreased Tₜ was due to interactions between the ELP chain and solvent-exposed hydrophobic regions in tendamistat, whereas, for the highly soluble thioredoxin, these hydrophobic interactions were negligible. Moreover, with highly soluble proteins only a small perturbation of Tₜ relative to the free ELP is likely to be introduced upon fusion with an ELP tag.

In order to demonstrate fundamental concepts, a gene encoding an ELP sequence was synthesized and ligated into two fusion protein constructs. In the first construct, an ELP sequence was fused to the C-terminus of E. coli thioredoxin, a 109 residue protein that is commonly used as a carrier to increase the solubility of target recombinant proteins. In the second, more complex construct, tendamistat, a 77 residue protein inhibitor of α-amylase, was fused to the C-terminus of a thioredoxin-ELP fusion, forming a ternary fusion.

Previous studies by Urry and colleagues have shown that two ELP-specific variables, guest residue(s) composition (i.e., identity and mole fraction of X in the VPGXG monomer) and chain length of the ELP profoundly affect the transition temperature, and thereby permit the peptide active therapeutic agent-ELP construct to be characterized by the Tₜ.

A gene was synthesized encoding an ELP sequence (SEQ ID NO: 13) with guest residues valine, alanine, and glycine in the ratio 5:2:3, with a predicted Tₜ of ˜40° C. in water. The synthetic gene, which encoded 10 VPGXG pentapeptide repeats (the “10-mer”), was oligomerized up to 18 times to create a library of genes encoding ELPs with precisely-specified molecular weights (MWs) ranging from 3.9 to 70.5 kDa. Thioredoxin was expressed as an N-terminal fusion with the 10-, 20, 30-, 60-, 90-, 120-, 150-, and 180-mer ELP sequences, and tendamistat was expressed as a C-terminal fusion to thioredoxin/90-mer ELP.

The FPs were expressed in E. coli and purified from cell lysate either by immobilized metal affinity chromatography (IMAC) using a (histidine)₆ tag present in the fusion protein or by inverse transition cycling (described below). The purified FP was cleaved with thrombin to liberate the target protein from the ELP. The ELP was then separated from the target protein by another round of inverse transition cycling, resulting in pure target protein. For each construct, the purified FP, target protein, and ELP were characterized by sodium dodecyl sulfate polyacrylamide gel electrophoresis (SDS-PAGE), which confirmed protein purity, verified completeness of thrombin cleavage, and showed that the migration of each protein was consistent with its predicted size (results not shown).

The inverse transition of the fusion protein so formed can be spectrophotometrically-characterized by monitoring solution turbidity as a function of temperature, due to aggregation of the ELP-containing fusion protein as it undergoes the transition. As the temperature is raised up to a critical temperature, the solution remains clear. Further increase in temperature results in a sharp increase in turbidity over a ˜2° C. range to a maximum value (OD₃₅₀˜2.0). The Tₜ, defined as the temperature at the midpoint of the spectrophotometrically-observed transition, is a convenient parameter to describe this process.

The inverse transition of free ELP, thioredoxin-ELP fusion, ELP-tendamistat fusion, and ternary thioredoxin-ELP-tendamistat fusion in PBS were studied. The Tₜ, was 51° C. for free ELP and 54° C. for the thioredoxin fusion, showing that the Tₜ is only slightly affected by fusion to thioredoxin. Thioredoxin-ELP produced by cleavage from the ternary tendamistat fusion had a higher Tₜ compared to thioredoxin-ELP produced directly (60° C. vs. 54° C.), presumably due to differences in the leader and trailer amino acid sequences immediately adjacent to the ELP sequence. The transition profiles of ELP-tendamistat and the thioredoxin-ELP-tendamistat were nearly identical, with a Tₜ of 34° C. Aggregation of the FPs was reversible, and the aggregates were resolubilized completely upon lowering the temperature below the Tₜ. However, resolubilization kinetics were slower for ELP-tendamistat and thioredoxin-ELP-tendamistat fusions, typically requiring 5 to 10 minutes versus only a few seconds for free ELP and thioredoxin-ELP. Thioredoxin and tendamistat controls exhibited no change in absorbance with increasing temperature, indicating that the thermally-induced aggregation observed for the fusion proteins was due to the inverse transition of the ELP carrier. Typically, the inverse transition of the fusion proteins was also slightly broader than that of free ELP, and small upper and lower shoulders were observed in their turbidity profiles.

In studies by Urry and colleagues, a decrease in Tₜ was observed with increasing chain length, and the effect of ELP MW on the inverse transition of FPs was also investigated. The Tₜ of a set of thioredoxin-FPs was determined as a function of the MW of the ELP carrier, which ranged from 12.6 to 71.0 kDa. The Tₜ's of the higher MW fusion proteins approached the design target temperature of 40° C. (42° C. for the 71 kDa ELP), while the Tₜ's for the lower MW fusions were significantly greater (e.g., 77° C. for the 12.6 kDa ELP).

In addition to ELP-specific variables that affect the Tₜ (i.e., guest residue composition and MW), the Tₜ can be further modulated for a given ELP by several extrinsic factors, such as the choice of solvent, ELP concentration, and ionic strength. Controlling the ionic strength, in particular, allows the Tₜ to be tuned over a 50° C. range, and thereby provides a convenient method to optimize the Tₜ of a given ELP for a specific application. Manipulating the solution temperature and ionic strength also provides experimental flexibility in inducing the inverse transition for a specific ELP by several methods: (1) by increasing the solution temperature above the Tₜ at a given ionic strength, (2) by increasing the ionic strength isothermally to reduce the Tₜ below solution temperature, or (3) by simultaneously changing the solution temperature and ionic strength.

The specific activity of the thioredoxin/60-mer FP, determined by an insulin reduction assay, was identical to that of commercially-available E. coli thioredoxin (results not shown), indicating that below the Tₜ, the ELP had no effect on thioredoxin activity. For the ternary thioredoxin-ELP-tendamistat fusion, an α-amylase inhibition assay showed that the thioredoxin/90-mer ELP carrier reduced the α-amylase inhibition activity of tendamistat by 2-fold (results not shown). However, after thrombin cleavage and purification of tendamistat from the thioredoxin-ELP carrier, the activity of purified tendamistat was indistinguishable from recombinant tendamistat, which was independently purified by IMAC.

The application of inverse transition cycling for protein purification requires that the phase transition of the ELP does not denature the target protein. The aggregation, resolubilization, and functional activity of the thioredoxin/60-mer ELP fusion upon thermally cycling in 1.5 M NaCl were therefore monitored. 1.5 M NaCl was added to the buffer simply to lower the Tₜ (from 62° C. in water to 27° C.) so that the FP would undergo its inverse transition in each thermal cycle between the experimentally-convenient temperatures of 24 and 35° C. Before commencing thermal cycling, the solution temperature of 24° C. was below the Tₜ of the thioredoxin-FP, and the protein solution exhibited no detectable turbidity. The thioredoxin activity of the fusion protein was initially assayed at this temperature to establish a baseline. Upon increasing the temperature to 35° C., the fusion protein aggregated, resulting in increased turbidity (OD₃₅₀˜2.0). After lowering the temperature to 24° C., the solution cleared completely, indicating that the fusion protein had resolubilized. An aliquot was removed and assayed for thioredoxin activity, which was found to be identical to the initial value. This thermal cycling process was repeated twice. No change in activity was observed at 24° C. after each thermal cycle, which confirmed that the small temperature change and the resulting aggregation/resolubilization had no effect on protein stability and function. In addition, resolubilization and recovery of the aggregated fusion protein was quantitative and complete after lowering the temperature to 24° C.

Six thioredoxin-FPs, where each fusion protein contained a C-terminal 30-, 60-, 90-, 120-, 150-, or 180-mer ELP tag, and the thioredoxin/90-mer ELP/tendamistat fusion protein were purified from cell lysate by inverse transition cycling, achieved by repeated centrifugation at conditions (i.e., NaCl concentration and temperature) alternating above and below the transition temperature.

Before purification, the induced E. coli were harvested from culture media by centrifugation, resolubilized in a low salt buffer (typically PBS), and lysed by ultrasonic disruption. After high-speed centrifugation to remove insoluble matter, polyethylenimine was added to the lysate to precipitate DNA, yielding soluble lysate. Inverse transition cycling was then initiated by adding NaCl and/or increasing the solution temperature to induce the inverse transition of the FP, causing the solution to become turbid as a result of aggregation of the FP. The aggregated fusion protein was separated from solution by centrifugation at a temperature greater than the Tₜ, and a translucent pellet formed at the bottom of the centrifuge tube. The supernatant, containing contaminating E. coli proteins, was decanted and discarded. The pellet was redissolved in a low ionic strength buffer at a temperature below the Tₜ of the ELP, and centrifuged at low temperature to remove any remaining insoluble matter. Although additional rounds of inverse transition cycling were undertaken, the level of contaminating proteins was below the detection limit of SDS-PAGE after a single round of inverse transition cycling.

A study of thioredoxin specific activity at each stage of purification of the thioredoxin/ELP fusion protein, as well as a determination of the total protein as estimated by BCA assay, showed that approximately 20% of the total protein in the soluble lysate (1) was precipitated in the first round of inverse transition purification (3), and the remaining soluble protein was decanted and discarded (2). The low thioredoxin activity measured in the supernatant, a portion of which is contributed by native E. coli thioredoxin, confirmed that this fraction primarily contained contaminating host proteins. The thioredoxin specific activity of the resolubilized protein approached that of commercially-available thioredoxin (data not shown), which confirmed that one round of inverse transition cycling resulted in complete purification. A second round of purification resulted in no detectable increase in thioredoxin specific activity (data not shown). The total thioredoxin activity after several rounds of inverse transition purification was experimentally-indistinguishable from that of the cell lysate, indicating negligible loss of target protein in the discarded supernatant. These results quantitatively confirmed the high purity and efficient recovery of the thioredoxin-FP, and further demonstrated that functional activity of thioredoxin is fully retained after undergoing several rounds of inverse transition cycling.

Protein yields for the thioredoxin fusion constructs were typically greater than 50 milligrams of purified fusion protein per liter culture. It was found that the total gravimetric yield of fusion protein decreased with increasing ELP length, with the 30-mer (MW=12.6 kDa) averaging ˜70 mg/L and the 180-mer (MW=71.0 kDa) averaging ˜50 mg/L. Expression levels of soluble tendamistat were slightly larger for the ternary thioredoxin-ELP-tendamistat fusion (45 mg/L ternary fusion, or 7 mg/L tendamistat) compared to its fusion with thioredoxin only (10 mg/L thioredoxin-tendamistat fusion, 4 mg/L tendamistat).

As described hereinabove, two recombinant proteins, thioredoxin and tendamistat, fused to an environmentally-responsive ELP sequence, were expressed and a gentle, one-step separation of these fusion proteins from other soluble E. coli proteins was achieved by exploiting the inverse transition of the ELP sequence. Thioredoxin and tendamistat were selected as target proteins because they exemplify two limiting scenarios of soluble protein expression: (1) the target protein over-expresses at high levels and is highly soluble (thioredoxin), and (2) the protein is expressed largely as insoluble inclusion bodies (tendamistat). However, proteins representative of this latter class must exhibit some level of expression as soluble protein to be purified by inverse transition cycling.

Thioredoxin is expressed as soluble protein at high levels in E. coli, and is a therefore a good candidate for determining whether the reversible, soluble-insoluble inverse transition of the ELP tag would be retained in a fusion protein. In contrast, tendamistat was selected as the other test protein because it is largely expressed as insoluble protein in inclusion bodies. Although fusion with thioredoxin promotes the soluble expression of target proteins, only 5-10% of over-expressed thioredoxin-tendamistat fusion protein was recovered as soluble and functionally-active protein.

The ELP polypeptide tag used for thermally-induced, phase separation of the target recombinant protein was derived from polypeptide repeats found in mammalian elastin. Because the phase transition of ELPs is the fundamental basis of protein purification by inverse transition cycling, specifying the transition temperature is the primary objective in the design of an ELP tag.

Previous studies by Urry and colleagues have shown that the fourth residue (X) in the polypentapeptide sequence, VPGXG, can be altered without eliminating the formation of the β-turn, a structure that is advantageous to the inverse transition. These studies also showed that the Tₜ is a function of the hydrophobicity of the guest residue. Therefore, by varying the identity of the guest residue(s) and their mole fraction(s), ELP copolymers can be synthesized that exhibit an inverse transition over a 0-100° C. range. Based on these results, an amino acid sequence was selected to result in a predicted Tₜ of ˜40° C. in water, so that the ELP carrier would remain soluble in E. coli during culture but could be aggregated by a small increase in temperature after cell lysis.

In addition to the amino acid sequence, it is known that Tₜ also varies with ELP chain length. The design therefore incorporated precise control of molecular weight by a gene oligomerization strategy so that a library of ELPs with systematically varied molecular weight could be synthesized. The Tₜ's of the higher molecular weight ELPs approached the target temperature, with an experimentally-observed Tₜ of 42° C. for the thioredoxin/180-mer fusion (at 25 μM in PBS). However, the Tₜ increased dramatically with decreasing MW. In low ionic strength buffers, the Tₜ's of the lower molecular weight ELPs are too high for protein purification, and would consequently require a high concentration of NaCl to decrease the Tₜ to a useful temperature. ELP chain length is also important with respect to protein yields. In addition to the decreased total yield of expressed fusion protein observed with increasing ELP MW, the weight percent of target protein versus the ELP also decreases as the MW of the ELP carrier increases. Therefore, the design of the ELP tags for purification preferably maximizes target protein expression by minimizing the ELP molecular weight, while retaining a target Tₜ near 40° C. through the incorporation of a larger fraction of hydrophobic guest residues in the ELP sequence.

The thioredoxin-ELP fusion as described hereinabove exhibited only a small increase in Tₜ (1-2° C.) compared to free ELP, while the tendamistat-ELP fusion displayed a more dramatic 15° C. reduction in Tₜ. This shift was identical for both the ternary (thioredoxin-ELP-tendamistat) and binary (ELP-tendamistat) constructs, indicating that the Tₜ shift is associated specifically with tendamistat. These observations suggested that the decreased Tₜ was due to interactions between the ELP chain and solvent-exposed hydrophobic regions in tendamistat, whereas, for the highly soluble thioredoxin, these hydrophobic interactions were negligible. Although this shift in Tₜ added complexity to the design of ELP carriers for inverse transition purification of proteins containing a significant fraction of exposed hydrophobic area, for highly soluble proteins only a small perturbation of Tₜ relative to the free ELP is likely to be introduced upon fusion with an ELP tag.

Standard molecular biology protocols were used for gene synthesis and oligomerization of the ELP tags. The synthetic gene for the 10-mer polypentapeptide VPGXG ELP was constructed from four 5′-phosphorylated, PAGE-purified synthetic oligonucleotides (Integrated DNA Technologies, Inc.), ranging in size from 86 to 97 bases. The oligonucleotides were annealed to form double-stranded DNA spanning the ELP gene with EcoRI and HindIII compatible ends. The annealed oligonucleotides were then ligated, using T4 DNA ligase, into EcoRI/HindIII linearized and dephosphorylated pUC-19 (NEB, Inc.). Chemically competent E. coli cells (XL1-Blue) were transformed with the ligation mixture, and incubated on ampicillin-containing agar plates. Colonies were initially screened by blue-white screening, and subsequently by colony PCR to verify the presence of an insert. The DNA sequence of a putative insert was verified by dye terminator DNA sequencing (ABI 370 DNA sequencer).

First, a 20-mer ELP gene was created by ligating a 10-mer ELP gene into a vector containing the same 10-mer ELP gene. The 20-mer gene was similarly combined with the original 10-mer gene to form a 30-mer gene. This combinatorial process was repeated to create a library of genes encoding ELPs ranging from 10-mer to 180-mer polypentapeptides. For a typical polymerization or oligomerization, the vector was linearized with PflMI and enzymatically dephosphorylated. The insert was doubly digested with PflMI and BglI, purified by agarose gel electrophoresis (Qiaex II Gel Extraction Kit, Qiagen Inc.), ligated into the linearized vector with T4 DNA ligase, and transformed into chemically competent E. coli cells. Transformants were screened by colony PCR, and further confirmed by DNA sequencing.

For the thioredoxin fusion proteins, pET-32b expression vector (Novagen Inc.) was modified to include an SfiI restriction site and a transcriptional stop codon downstream of the thioredoxin gene. For the ternary tendamistat fusion, a previously constructed pET-32a based plasmid containing a gene for a thioredoxin-tendamistat fusion was modified to contain an SfiI restriction site in two alternate locations, upstream or downstream of the thrombin recognition site. ELP gene segments, produced by digestion with PflMI and BglI, were then ligated into the SfiI site of each modified expression vector. Cloning was confirmed by colony PCR and DNA sequencing.

The expression vectors were transformed into the expression strains BLR(DE3) (for thioredoxin fusions) or BL21-trxB(DE3) (for tendamistat fusion) (Novagen, Inc.). Shaker flasks with 2× YT media, supplemented with 100 μg/ml ampicillin, were inoculated with transformed cells, incubated at 37° C. with shaking (250 rpm), and induced at an OD₆₀₀ of 0.8 by the addition of isopropyl α-thiogalactopyranoside (IPTG) to a final concentration of 1 mM. The cultures were incubated an additional 3 hours, harvested by centrifugation at 4° C., resolubilized in low ionic strength buffer (˜1/30 culture volume), and lysed by ultrasonic disruption at 4° C. The lysate was centrifuged at ˜20,000×g at 4° C. for 15 minutes to remove insoluble matter. Nucleic acids were precipitated by the addition of polyethylenimine (0.5% final concentration), followed by centrifugation at ˜20,000×g at 4° C. for 15 minutes. Soluble and insoluble fractions of the cell lysate were then characterized by sodium-dodecyl sulfate polyacrylamide gel electrophoresis (SDS-PAGE).

The thioredoxin-ELP fusions, which contained a (His)₆ tag, were purified by immobilized metal ion affinity chromatography (IMAC) using a nickel-chelating nitrilotriacetic derivatized resin (Novagen Inc.) or alternatively by inverse transition cycling. The tendamistat-ELP fusion was purified exclusively by inverse transition cycling. For purification by inverse transition cycling, FPs were aggregated by increasing the temperature of the cell lysate to ˜45° C. and/or by adding NaCl to a concentration ˜2 M. The aggregated fusion protein was separated from solution by centrifugation at 35-45° C. at 10-15,000×g for 15 minutes. The supernatant was decanted and discarded, and the pellet containing the fusion protein was resolubilized in cold, low ionic strength buffer. The resolubilized pellet was then centrifuged at 4° C. to remove any remaining insoluble matter.

The optical absorbance at 350 nm of ELP fusion solutions were monitored in the 4-80° C. range on a Cary 300 UV-visible spectrophotometer equipped with a multi-cell thermoelectric temperature controller. The Tₜ was determined from the midpoint of the change in optical absorbance at 350 nm due to aggregation of FPs as a function of temperature at a heating or cooling rate of 1.5° C. min⁻¹.

SDS-PAGE analysis used precast Mini-Protean 10-20% gradient gels (BioRad Inc.) with a discontinuous buffer system. The concentration of the fusion proteins was determined spectrophotometrically using calculated extinction coefficients. Total protein concentrations were determined by BCA assay (Pierce). Thioredoxin activity was determined by a colorimetric insulin reduction assay. Tendamistat activity was determined by a colorimetric α-amylase inhibition assay (Sigma).

ELP-GFP fusion proteins were also synthesized, wherein the ELP 90-mer and 180-mer were fused either N-terminal or C-terminal to green fluorescent protein (GFP) or its variant—blue fluorescent protein (BFP). All fusion polypeptides exhibited a reversible inverse transition as characterized by UV-vis spectrophotometric measurement of turbidity as a function of temperature, as well as temperature dependent fluorescence measurement. The inverse transition of the GFP-ELP and BFP-ELP fusions, was used to purify these fusion proteins to homogeneity by ITC, and was verified by SDS-PAGE and Coomassie staining.

Standard molecular biology protocols were further used for synthesis and polymerization/oligomerization of the ELP genes with reduced ELP molecular weight (Ausubel, et al.). Monomer genes for two ELP sequences were utilized in this example.

The first, ELP1 [V₅A₂G₃-10] encoded ten Val-Pro-Gly-Xaa-Gly repeats where Xaa was Val, Ala, and Gly in a 5:2:3 ratio (SEQ ID NO: 13), respectively. The second monomer, ELP1 [V-5] (SEQ ID NO: 14), encoded five Val-Pro-Gly-Val-Gly pentapeptides (i.e., Xaa was exclusively Val). The coding sequence for the ELP1 [V-5] monomer gene was: 5′-GTGGGTGTTCCGGGCGTAGGTGTCCCAGGTGTGGGCGTACCGGGCGTTGGTGTTCCTG GTGTCGGCGTGCCGGGC-3′ (SEQ ID NO: 15). The monomer genes were assembled from chemically synthesized, 5′-phosphorylated oligonucleotides (Integrated DNA Technologies, Coralville, Iowa), and ligated into a pUC19-based cloning vector. A detailed description of the monomer gene synthesis is presented elsewhere.

The monomer genes for both ELP sequences, ELP1 [V₅A₂G₃-10] and ELP1 [V-5], were seamlessly oligomerized by tandem repetition to encode libraries of increasing ELP molecular weight. A detailed description of the gene oligomerization, using a methodology termed “recursive directional ligation,” is presented elsewhere. Briefly, an ELP gene segment (the monomer gene initially and larger multiples of the monomer in later rounds) is excised by restriction digest from its vector, purified, and ligated into a second cloning vector containing the same or a different ELP gene segment, thereby concatenating the two gene segments. This process can be repeated recursively, doubling the gene length with each round.

Different ELP constructs are distinguished here using the notation ELPk [XᵢYⱼ-n], where k designates the specific type of ELP repeat unit, the bracketed capital letters are single letter amino acid codes and their corresponding subscripts designate the relative ratio of each guest residue X in the repeat units, and n describes the total length of the ELP in number of the pentapeptide repeats. The two ELP constructs central to the present example are ELP1 [V₅A₂G₃-90] (35.9 kDa) (SEQ ID NO: 16) and ELP1 [V-20] (9.0 kDa) (SEQ ID NO: 17).

To produce the thioredoxin fusion proteins, genes encoding ELP1 [V₅A₂G₃-90] and ELP1 [V-20] were excised from their respective cloning vectors and separately ligated into a pET-32b expression vector (Novagen, Madison, Wis.), which had been previously modified to introduce a unique Sfi I site located 3′ to the thioredoxin gene, a (His)₆ tag, and a thrombin protease cleavage site. The modified pET32b vector encoding free thioredoxin with no ELP tag (“thioredoxin(His₆)”) and the two expression vectors encoding each fusion protein (“thioredoxin-ELP1 [V₅A₂G₃-90]” and “thioredoxin-ELP1 [V-20]”) were transformed into the BLR(DE3) E. coli strain (Novagen).

For quantitative comparison of the protein expression levels and purification yields, the three constructs were each expressed and purified in parallel. For each sample (four samples each of thioredoxin(His₆), thioredoxin-ELP1 [V-20], and thioredoxin-ELP1 [V₅A₂G₃-90]), a 2 ml starter culture (CircleGrow media, Qbiogene, Carlsbad, Calif., supplemented with 100 μg/ml ampicillin) was inoculated with a stab taken from a single colony on a freshly streaked agar plate, and incubated overnight at 37° C. with shaking at 300 rpm. To remove B-lactamase from the media, the cells were then pelleted from 500 μl of the confluent overnight culture by centrifugation (2000×g, 4° C., 15 min), resuspended in fresh media wash, and repelleted. After a second resuspension in fresh media, the cells were used to inoculate 50 ml expression cultures in 250 ml flasks (CircleGrow media with 100 μg/ml ampicillin).

The culture flasks were incubated at 37° C. with shaking at 300 rpm. Growth was monitored by the optical density at 600 nm, and protein expression was induced at OD₆₀₀=1.0 by the addition of isopropyl β-thiogalactopyranoside (IPTG) to a final concentration of 1 mM. After a further 3 hours of culture, the cells were harvested from 40 ml by centrifugation (2,000×g, 4° C., 15 min), resuspended in 2 ml of IMAC binding buffer (5 mM imidazole, 500 mM NaCl, 20 mM Trix-HCl, pH 7.9) for thioredoxin(His₆) or PBS (137 mM NaCl, 2.7 mM KCl, 4.2 mM Na₂HPO₄, 1.4 mM KH₂PO₄, pH 7.3) for thioredoxin-ELP1 [V-20] and thioredoxin-ELP1 [V₅A₂G₃-90], and stored frozen at −20° C. until purified. The culture density at harvest was measured by OD₆₀₀, after 1:10 dilution in fresh buffer. The amount of plasmid DNA at harvest was quantified by UV-visible spectrophotometry following plasmid isolation (plasmid miniprep spin kit, Qiagen, Valencia, Calif.).

As a control for ITC purification of the thioredoxin-ELP fusion proteins, free thioredoxin was purified using standard IMAC protocols. Briefly, the thawed cells were transferred to iced 15 ml centrifuge tubes and lysed by ultrasonic disruption (Fisher Scientific 550 Sonic Dismembrator using a microtip). After transferring to 1.5 ml micro centrifuge tubes, the E. coli lysate was centrifuged (16,000×g, 4° C., 30 min) to remove the insoluble cellular debris. 1 ml of the soluble cell lysate was loaded by gravity flow onto a column packed a 1 ml bed of nitrilotriacetic acid resin that had been charged with 5 ml of 50 mM NiSO₄.

After the column was washed with 15 ml of IMAC binding buffer, thioredoxin(His₆) was eluted in 6 ml of IMAC binding buffer supplemented with 250 mM imidazole. Imidazole was removed from the eluent by dialysis against a low salt buffer (25 mM NaCl, 20 mM Tris-HCl, pH 7.4) overnight using a 3,500 MWCO membrane. The IMAC purification was monitored by SDS-PAGE using precast 10-20% gradient gels (BioRad Inc., Hercules, Calif.) with a discontinuous buffer system.

The yield of the purified thioredoxin(His₆) was determined by spectrophotometry, using a molar extinction coefficient of thioredoxin modified to include the absorption of the single Trp residue present in the C-terminal tag (ε₂₈₀=19870 M⁻¹cm⁻¹ for thioredoxin(His₆) and all thioredoxin-ELP fusion proteins, independent of ELP molecular weight.

In a typical purification by ITC, the thawed cells were transferred to iced 15 ml centrifuge tubes and lysed by ultrasonic disruption (Fisher Scientific 550 Sonic Dismembrator with a microtip). After transferring to 1.5 ml micro centrifuge tubes, the E. coli lysate was centrifuged at 4° C. for 30 min to remove the insoluble cellular debris. (All centrifugation steps during purification by ITC were performed at 16,000×g in Eppendorf 5415C microcentrifuges.)

Polyethylenimine was added (to 0.5% w/v) to the decanted supernatant of the cell lysate to precipitate nucleic acids, which were removed by an additional 20 min centrifugation at 4° C. The supernatant was retained, and the ELP phase transition was induced by increasing the NaCl concentration by 1.3 M. The aggregated fusion protein was separated from solution by centrifugation at 33° C. for 5 min, which resulted in the formation of translucent pellet at the bottom of the tube.

The supernatant was decanted and discarded, and the pellet containing the fusion protein was redissolved in an equal volume of PBS at 4° C. Any remaining insoluble matter was removed by a final centrifugation step at 4° C. for 15 min, and the supernatant containing the purified fusion protein was retained. The progression of fusion protein purification was monitored by SDS-PAGE, and the protein concentrations were determined by spectrophotometry, as described above for MAC purification.

Thioredoxin was liberated from its ELP fusion partner using thrombin protease (Novagen), which cleaved the fusion protein at a recognition site located between thioredoxin and the ELP tag. The thrombin proteolysis reaction was allowed to proceed overnight at room temperature in PBS Using ˜10 units of thrombin per μmol of fusion protein, which was typically at a concentration of ˜100 μM. Free ELP was then separated from the cleaved thioredoxin by another round of ITC, this time retaining the supernatant that contained the product thioredoxin.

The inverse transition can be monitored by assaying solution turbidity photometrically as a function of temperature, taking advantage of the fact that increase in temperature beyond a critical point results in a sharp increase in turbidity over an approximately 2° C. range to a maximum value (OD₃₅₀ approximately 2.0), because of aggregation of the ELP. The temperature at 50% maximal turbidity, T_{b}, is a convenient parameter for quantitatively monitoring the aggregation process.

The temperature-dependent aggregation behaviors of the thioredoxin-ELP fusion proteins were characterized by measuring the optical density at 350 nm as a function of temperature. Fusion proteins at concentrations typical of those found in the E. coli lysate during protein purification (160 μM for thioredoxin-ELP1 [V-20] and 40 μM for thioredoxin-ELP1 [V₅A₂G₃-90]) were heated or cooled at a constant rate of 1° C. min⁻¹ in a Cary Bio-300 UV-visible spectrophotometer (Varian Instruments, Walnut Creek, Calif.), which was equipped with a thermoelectric temperature-controlled multicell holder. The experiments were performed in PBS variously supplemented with additional NaCl. The ELP Tₜ was defined as the temperature at which the optical density reached 5% of the maximum optical density at 350 nm.

Dynamic light scattering (DLS) was used to monitor the particle size distribution of the thioredoxin-ELP fusion proteins as a function of temperature and NaCl concentration. Samples were prepared to reflect the protein and solvent compositions used in the turbidity measurements described above, and were centrifuged at 4° C. and 16,000×g for 10 minutes to remove air bubbles and insoluble debris. Prior to particle size measurement, samples were filtered through a 20 nm Whatman Anodisc filter at a temperature below the Tₜ.

Autocorrelation functions were collected using a DynaPro-LSR dynamic light scattering instrument (Protein Solutions, Charlottesville, Va.) equipped with a Peltier temperature control unit. Analysis was performed using Protein Solutions' Dynamics software version 5.26.37 using its regularization analysis for spherical particles. Light scattering data were collected at regular temperature intervals (either 1 or 2° C.) as solutions were heated from 20° to 60° C. Data were collected at each temperature by ramping the cell up to the temperature of interest, allowing the sample temperature equilibrate for at least 1 minute, and collecting 10 measurements, each with a 5 second collection time.

The inverse transition of each thioredoxin-ELP fusion protein in solution was characterized by monitoring the optical density at 350 nm as a function of temperature. Because different NaCl solutions are routinely used during ITC purification to depress the Tₜ or isothermally trigger the inverse transition, turbidity profiles were obtained for 40 μM thioredoxin-ELP1 [V₅A₂G₃-90] and 160 μM thioredoxin-ELP1 [V-20] in PBS and in PBS with an additional 1M, 2M, and 3M NaCl.

Optical density at 350 nm as a function of temperature was assessed for solutions of the thioredoxin-ELP fusion proteins. The turbidity profiles were obtained for thioredoxin-ELP1 [V-20] (solid lines) and thioredoxin-ELP1 [V₅A₂G₃-90] (dashed lines) in PBS, and in PBS supplemented with 1, 2, and 3 M NaCl, while heating at a rate of 1° C. min⁻¹. The concentration of thioredoxin-ELP1 [V₅A₂G₃-90] was 40 μM in each of the four PBS solutions, and that of thioredoxin-ELP1 [V-20] was 160 μM, which matched the typical concentration of each protein in the soluble cell lysate during ITC purification. All solutions showed a rapid rise in turbidity as they were heated through the Tₜ, but with continued heating beyond the Tₜ, the thioredoxin-ELP1 [V-20] solutions eventually became less turbid while the thioredoxin-ELP1 [V₅A₂G₃-90] solutions remained consistently turbid. All solutions of thioredoxin-ELP1 [V₅A₂G₃-90] cleared fully upon cooling the solution to below the Tₜ. However, solutions of ELP1 [V-20] cleared reversibly only if the solutions were not heated to above ˜55° C., suggesting thermal denaturation of the thioredoxin fusion protein occurred above this temperature.

The protein concentrations were chosen as typical of the concentrations obtained for each fusion protein in the soluble fraction of E. coli lysate, the stage at which the ELP inverse transition is first induced during ITC purification. Turbidity profiles obtained directly in the E. coli soluble cell lysate, supplemented with 1 and 2 M NaCl, were indistinguishable from the corresponding profiles determined for the thioredoxin fusion protein as described in the preceding paragraph. (Turbidity profiles were not routinely obtained in E. coli lysate because of the potential for turbidity arising from thermal denaturation of E. coli proteins, which could not be differentiated from turbidity arising from aggregation of the ELP fusion protein.) Turbidity profiles were also obtained for each fusion protein in PBS with 1.3 M salt, which matched the conditions used for the ITC purification described below.

The heating and cooling turbidity profiles for the solution conditions used in ITC purification were determined for solutions of thioredoxin-ELP1 [V-20] (solid lines) and thioredoxin-ELP1 [V₅A₂G₃-90] (dashed lines) at lysate protein concentrations in PBS with 1.3 M NaCl, corresponding to ITC conditions used for the quantitative comparison of expression and purification. These conditions were chosen so that the maximum turibidity of the thioredoxin-ELP1 [V-20] solution occurred at the centrifugation temperature of 33° C. The solutions were heated and cooled at 1° C. min⁻¹. The slight path differences between the heating and cooling curves that were observed were primarily due to slow settling of the aggregates over time at temperatures above Tₜ, and to the slower kinetics of disaggregation versus aggregation as the solutions are cooled to below Tₜ.

The thermally induced aggregation behavior of thioredoxin-ELP1 [V₅A₂G₃-90] was similar to that of free ELPs. All four salt concentrations, as the temperature of the thioredoxin-ELP1 [V₅A₂G₃-90] solutions was increased, remain clear until they reach the ELP Tₜ, at which point the turbidity sharply increased. This occurred at 51, 31, 15, and 4° C. in PBS with 0, 1, 2, and 3 M added NaCl, respectively. A free thioredoxin control solution exhibited no change in turbidity with increasing temperature over this temperature range, indicating that the thermally induced aggregation observed was due to the inverse transition of the ELP tag (results not shown). As these solutions were heated further beyond the Tₜ, the turbidity level remained essentially constant, and was only slightly reduced by settling of the aggregates over time. Upon cooling to below the Tₜ, the aggregates resolubilize and the optical density returned to zero, showing that the inverse transition of the ELP1 [V₅A₂G₃-90] fusion protein was completely reversible. While increasing the NaCl concentration markedly decreases the Tₜ, salt has no measurable effect on the maximum optical density, on the general shape of the turbidity profiles, or on the reversibility of the aggregation.

In contrast, the phase transition behavior of thioredoxin-ELP1 [V-20] was considerably more complex than for the thioredoxin-ELP1 [V₅A₂G₃-90] fusion protein and free ELPs. Although the initial rapid rise in turbidity at the Tₜ (33, 17, and 4° C. in PBS supplemented with 1, 2, and 3 M NaCl, respectively) was similar to the other ELP constructs, the maximum turbidity observed with each of the thioredoxin-ELP1 [V-20] solutions increased with increasing salt concentration. Furthermore, increases in temperature beyond the Tₜ eventually resulted in a significant decrease in turbidity. This decrease was reversible; if the solution was cooled after heating to the point of decreased turbidity, the turbidity again increased. Because the clearing phenomenon is a reversible function of temperature, it was concluded that a second, thermodynamically driven molecular rearrangement occurs with increasing temperature after the initial ELP aggregation event at Tₜ.

Another unique feature of the thioredoxin-ELP1 [V-20] turbidity profiles was a second increase in turbidity beginning at ˜55° C., which may have been due to aggregation arising from the irreversible thermal denaturation of thioredoxin. Samples heated to less than 55° C. reversibly cleared upon cooling to below the Tₜ, whereas samples that are heated to above 55° C., for salt concentrations of 1 M and greater, remained turbid even upon cooling to below the Tₜ (not shown). This phenomenon appeared to be unique to the thioredoxin-ELP1 [V-20] fusion protein, as solutions of free thioredoxin and of its fusion proteins to larger ELPs were stable to much higher temperatures (results not shown). No inverse transition was observed for thioredoxin-ELP1 [V-20] in PBS below 60° C., however, with added salt the Tₜ was depressed so that it occurred below the denaturation temperature in the PBS+1, 2, and 3 M NaCl solutions.

The sizes of the fusion protein particles were measured using DLS as a function of temperature to determine the effect of temperature and salt on the particle size distribution (radius of hydration, Rₕ) of 40 μM thioredoxin-ELP1 [V₅A₂G₃-90] in PBS, PBS+1 M NaCl, and PBS+2 M NaCl. The sizes of thioredoxin-ELP1 [V₅A₂G₃-90] particles in PBS, PBS with 1M added NaCl, and PBS with 2M added NaCl indicated that the sharp increase in turbidity at the Tₜ resulted from the conversion of monomers with hydrodynamic radii (Rₕ) of 5.9±3.9 nm to aggregates with Rₕ of 180±62 nm. These aggregates grew with temperature until reaching a stable Rₕ of 2.2±3.8 μm approximately 6° C. above the onset of the transition. Although the Tₜ was depressed by the addition of NaCl, the sizes of both monomers and fully formed aggregates were not significantly affected by either the salt concentration or the temperature (outside the range immediately adjacent to the Tₜ), providing a rationale for the steady-state turbidity above the inverse Tₜ. The temperature at the onset of large aggregate formation closely matched the Tₜ determined by the turbidity measurements for corresponding solution conditions.

The corresponding quantitative breakdown of scattered intensity attributed to each type of particle was also studied for each of the salt concentrations investigated. When two or more phases coexist over a given temperature range, these data show shifts in the relative particle populations. It should be noted that the intensity attributed to a particular population was not linearly correlated with the mass of that population, and that calculating the relative masses of multiple particles was complicated by changes in packing density that would likely accompany the inverse phase transition. Without a more detailed understanding of how temperature affects the packing density of ELPs and ELP fusion proteins, it was not possible to make a reasonable estimate for the mass attributed to each type of particle. Given these quantitative limitations, this data nonetheless showed that at the Tₜ the amount of scattered light attributed to the aggregate dramatically increased at the expense of the monomer.

The data also reflected the occasional presence of both an unidentified small particle (with apparent Rₕ=17±31 nm, albeit highly variable) and an extremely large aggregate (with apparent Rₕ=74±55 μm) coexisting with the 2 μm aggregates. It is unlikely that the small particle is a true component of the aggregate suspension; rather, its presence reflects an artifact in the regularization algorithm resulting from noise in the autocorrelation function. Assignment as an analysis artifact is supported by the small particle's highly variable size and by its inconsistent presence at temperatures above the transition. Likewise, because its apparent size is much larger than can be discerned by the DLS instrument, it is also unlikely that the extremely large aggregate predicted from the data analysis represented a true species in suspension. Rather, the scattering attributed to this species may result from the coordinated slow movements of a network of smaller particles.

In contrast to thioredoxin-ELP1 [V₅A₂G₃-90], the smaller thioredoxin-ELP1 [V-20] fusion protein showed a more complicated temperature-dependent particle size distribution, which was consistent with its more complex turbidity profile.

The effect of temperature on the particle size distribution of ELP1 [V-20] in PBS+1 M NaCl and PBS+2 M NaCl was studied. The clearing in turbidity when the temperature was increased beyond Tₜ coincided with the shifting of mass from large aggregates to a new, smaller particle (Rₕ=12 nm).

The effects of salt and temperature on the distribution of the particle Rₕ and the corresponding contribution of each particle population to scattered intensity of 160 μM thioredoxin-ELP1 [V-20] in PBS with 1M and 2M added NaCl, was likewise studied. For thioredoxin-ELP1 [V-20] with 1M added salt monomers with Rₕ of 5.9±5.1 nm were converted to aggregates with Rₕ of 140±79 nm at 30° C., corresponding to a small shoulder that preceded a rapid increase in turbidity at Tₜ. Above 30° C., aggregates grew with increasing temperature (up to Rₕ=1.5±0.98 μm at 40° C.), which was consistent with the rapid increase in turbidity observed starting at 33° C. Similar to the aggregation behavior of the large fusion protein, at temperatures greater than 40° C. thioredoxin-ELP1 [V-20] in PBS with 1M added NaCl showed the presence of very large aggregates (apparent Rₕ=64±67 μm) that may reflect the coordinated slow movements of a network of smaller particles.

However, unlike the larger fusion protein, thioredoxin-ELP1 [V-20] also showed the consistent presence of a previously unobserved small particle at temperatures above 40° C. This particle had a Rₕ of 12±4.9 nm, which was roughly twice that of the monomer. Yet, relative to its mean Rₕ, its variability was only one half that of the monomer. The size, consistency, and continuous presence of this particle above 40° C. indicated that it was neither an analysis artifact resulting from noise in the autocorrelation function nor was it resolvated monomer. The 12 nm particle appeared to form at the expense of mass in the aggregates initially present above Tₜ, as evidenced by the reduction in size and scattering intensity of the larger aggregates (Rₕ=200±210 nm) when the 12 nm particles were present.

A similar 12 nm particle was observed when the NaCl concentration was increased to 2 M. At this NaCl concentration, the Tₜ was lowered to 17° C. as determined by the turbidity measurements. This temperature range was limited at lower temperatures by the condensation of water vapor on the sample cuvette. Therefore, between 20° C. and 30° C., the thioredoxin-ELP1 [V-20] had already transitioned into stable aggregates with average Rₕ of 2.4±1.7 μm. As the samples was heated beyond ˜36° C., the Rₕ of the aggregates gradually decreased in size to 230±170 nm and 12 nm particles (Rₕ=12±4.7 nm) appeared. The percentage of scattered light attributable to the 12 nm particles also gradually increased at the expense of the shrinking larger aggregates.

Thioredoxin-ELP1 [V-20] and thioredoxin-ELP1 [V₅A₂G₃-90] were each purified by ITC from the soluble fraction of lysed E. coli cultures, and thioredoxin(His₆) was purified by IMAC as a control having no ELP tag. The inverse transition was induced by the addition of 1.3 M NaCl, and the centrifugation was carried out at 33° C. The smaller ELP1 [V-20] tag was successfully used to purify the fusion protein by ITC to homogeneity, with a yield and purity similar to that of the free thioredoxin purified by a conventional affinity chromatography method.

Note that the ELP tag was not stained by Coomassie, and therefore only the thioredoxin portion of the fusion protein was visible in stained gels. Qualitative comparison of the expression levels in the soluble cell lysate for thioredoxin-ELP1 [V-20] and thioredoxin-ELP1 [V₅A₂G₃-90] clearly showed that truncating the size of the ELP tag from 36 kDa to 9 kDa greatly enhanced the expression yield of the thioredoxin. Furthermore, thioredoxin-ELP1 [V-20] was expressed to a level qualitatively comparable to that of free thioredoxin. SDS-PAGE analysis also showed that there was no detectable loss to the insoluble fraction of the cell lysate for any the target proteins (results not shown).

For the ITC purifications, the ELP phase transition was triggered by adding 1.3 M additional NaCl and increasing the solution temperature to above ˜33° C. The cell lysates became turbid as a result of aggregation of the thioredoxin-ELP fusion proteins, which were then separated from solution by centrifugation at ˜33° C. to form a translucent pellet at the bottom of the centrifuge tube. SDS-PAGE showed that most contaminating E. coli proteins were retained in the decanted supernatant. The pellets were dissolved in PBS at ˜4° C., and centrifuged at low temperature (˜12° C.) to remove any remaining insoluble matter. The supernatants containing purified thioredoxin-ELP fusion proteins were retained. Finally, purified, free thioredoxin was obtained after cleavage of each fusion protein by thrombin at the encoded recognition site located between thioredoxin and the ELP tag, followed by a second round of ITC to remove the ELP tag from solution. Here, thrombin was retained with the target thioredoxin in the supernatant (although it was below the detection limit of Coomassie staining), however a thrombin-ELP fusion could be developed that would be removed after cleavage along with the free ELP.

These SDS-PAGE results clearly showed that thioredoxin can be purified by ITC to homogeneity, as ascertained by Coomassie staining, using the shorter, 9 kDa ELP1 [V-20]. However, differences were observed in the purification efficiency of the two ELP fusion proteins under these conditions, as qualitatively ascertained by SDS-PAGE. Recovery of thioredoxin-ELP1 [V₅A₂G₃-90] by ITC from the soluble cell lysate was essentially complete, whereas a small but significant fraction of thioredoxin-ELP1 [V-20] remained in the discarded supernatant. The level of purity obtained by ITC with the ELP1 [V-20] tag was qualitatively as good or better than that obtained by IMAC purification of the free thioredoxin, although with IMAC purification there was no detectable loss of the target protein in the column flow-through.

Using UV-visible spectrophotometry, the yield of each protein recovered by ITC or IMAC purification was quantified. Although these data described the amount of protein recovered after purification, SDS-PAGE results suggested that this quantity was nearly equal to expression yield in the soluble lysate. For this analysis, four cultures were grown in parallel under identical conditions for each of the three protein constructs. For experimental convenience, these data were obtained for 50 ml cultures, and extrapolated to yield per liter of culture. Purification of separate 1 liter cultures confirmed that the actual yields closely matched the extrapolated values (data not shown).

The total yields of thioredoxin(His₆), thioredoxin-ELP1 [V-20], and thioredoxin-ELP1 [V₅A₂G₃-90] from the 50 ml test cultures were determined, extrapolated to milligrams per liter of culture (mean±SD, n=4). The separate contributions of the ELP tag and thioredoxin to the yield, as calculated using their respective mass fractions of the fusion protein, were also determined for comparison. With all other experimental conditions identical, reducing the ELP tag from 36 (thioredoxin-ELP1 [V₅A₂G₃-90]) to 9 kDa (thioredoxin-ELP1 [V-20]) resulted in a near four-fold increase in the yield of the target thioredoxin.

The data showed that decreasing the molecular weight of the ELP tag can dramatically increase the yield of thioredoxin. Under experimentally identical conditions of E. coli culture, decreasing the ELP tag size from 36 kDa in thioredoxin-ELP1 [V₅A₂G₃-90] to 9 kDa in thioredoxin-ELP1 [V-20] increased the yield of fusion protein by 70% (82±12 mg/L versus 137±21 mg/L, respectively; P<0.005, unpaired t test). Furthermore, since truncating the size of the ELP tag reduced its mass fraction in the fusion protein, the target protein thioredoxin (i.e., if separated from the fusion protein at the thrombin cleavage site) constituted a larger fraction of the mass in the fusion protein yield. Thus, the yield of thioredoxin was 365% greater using the smaller tag (23±3.3 mg/L versus 83±12 mg/L for the larger and smaller tags, respectively; P<0.0001). This yield of thioredoxin obtained by ITC using the 9 kDa tag was statistically indistinguishable from that obtained for thioredoxin expressed without an ELP tag and purified using IMAC (93±13 mg/L; P>0.25).

These results corroborated the SDS-PAGE results since the relative yields of thioredoxin correlated with the expression levels observed in the cell lysate. The yield of the ELP tag was the same for both fusion proteins (59±8.6 mg/L for thioredoxin-ELP1 [V₅A₂G₃-90] and 54±8.1 mg/L for thioredoxin-ELP1 [V-20]; P>0.4). This was consistent with previous observations that the gravimetric yield of the ELP tag in thioredoxin fusion proteins was essentially constant with respect to ELP molecular weight within the ELP1 [V₅A₂G₃-90]] family of polypeptides ranging from 24 to 72 kDa.

To demonstrate the relationship between purification efficiency and ITC solution conditions, ITC purification of the thioredoxin-ELP1 [V-20] fusion protein was repeated using different combinations of salt concentration and centrifugation temperature.

SDS-PAGE analysis of the effect of NaCl concentration and centrifugation temperature on purification of thioredoxin-ELP[V-20] by ITC was carried out (SL=soluble cell lysate; S=supernatant after inverse transition of fusion protein and centrifugation to remove aggregated target protein; and P=redissolved pellet containing the purified fusion protein, after dissolution in PBS). The molar NaCl concentration and centrifugation temperature for each purification was noted. Although a high level of purity was achieved in each case, selection of an appropriate NaCl concentration and centrifugation temperature is critical to achieve complete purification efficiency.

When PBS with 1 M NaCl combined with centrifugation at 49° C. was used for ITC purification, the majority of the target fusion protein was lost in the discarded supernatant. When PBS plus 2 M NaCl and a centrifugation temperature of 33° C. was used, more than half of the target protein was captured by centrifugation. Finally, using PBS with 3 M NaCl and centrifugation at 12° C., the vast majority of the target protein was successfully purified. Although the target protein was purified to homogeneity in each of these examples, these results showed that selection of salt concentration and temperature was an important factor influencing the efficiency of ITC purification.

The objective of this example was to produce an ELP tag for ITC purification that was reduced in size relative to those previously reported, and to characterize the effect of this reduction on expression levels and on purification efficiency. In a prior effort, a first generation of ELP purification tags was developed based on a ELP1 [V₅A₂G₃-10] monomer sequence. This sequence was recursively oligomerized to create a library of synthetic genes encoding ELPs with molecular weights ranging from 4 kDa (ELP1 [V₅A₂G₃-10]) to 71 kDa (ELP1 [V₅A₂G₃-180]). This particular guest residue composition was selected based on previous studies of Urry et al., and ELPs with this composition were predicted to exhibit a Tₜ of ˜40° C. for molecular weights of ˜100 kDa in water. A 40° C. Tₜ was targeted so that the fusion proteins would remain soluble during culture at 37° C., but could be induced to reversibly aggregate through the ELP phase transition by a modest increase in salt concentration or solution temperature.

Although the Tₜ's of the higher molecular weight constructs approached 40° C. (Tₜ=42° C. for the thioredoxin-ELP1 [V₅A₂G₃-180], with MW_{ELP}=71 kDa, in PBS at 25 μM), the Tₜ of the thioredoxin-ELP1 [V₅A₂G₃] fusion proteins increased dramatically with decreasing molecular weight (Tₜ=77° C. for thioredoxin-ELP1 [V₅A₂G₃-30], with MW_{ELP}=13 kDa, under the same conditions). The high Tₜ's of the lower molecular weight ELPs required the addition of a very high concentration of NaCl (>3 M) to reduce their Tₜ to a useful temperature (e.g., 20-40° C.), which precluded their general use for purification by ITC because of the potential for salt-induced denaturation of target proteins. Although the larger ELP1 [V₅A₂G₃] polypeptides were successfully used to purify thioredoxin and second model target protein, tendamistat, it was observed that the yield of the fusion protein was significantly decreased as the ELP1 [V₅A₂G₃] chain length was increased.

These observations motivated the redesign of the ELP expression tag in the above experiment to reduce the size of the ELP expression tag while also depressing its Tₜ, so that lower molecular weight ELP tags would exhibit a Tₜ near 40° C. at more moderate NaCl concentrations. The second monomer gene, which was newly synthesized for this study, encoded a five pentamer ELP sequence where the fourth guest residue was exclusively Val (ELP1 [V-5]). Because the Val present in ELP1 [V] was more hydrophobic than the Ala and Gly present in ELP1 [V₅A₂G₃], thioredoxin-ELP1 [V] fusion proteins were predicted to have a Tₜ of 40° C. at smaller ELP molecular weights than for thioredoxin-ELP1 [V₅A₂G₃] fusions.

The ELP1 [V-20] sequence (four tandem repeats of the ELP1 [V-5] gene) was selected from a library of ELP1 [V-5] oligomers for further characterization at a ITC purification tag due to the empirical observation of its Tₜ near 40° C. at lysate protein concentration with moderate (1 M) NaCl. In the present example, the thioredoxin-ELP1 [V-20] construct (MW_{ELP}=9 kDa) was compared to the previously described thioredoxin-ELP1 [V₅A₂G₃-90] construct (MW_{ELP}=36 kDa) because the two fusion proteins had very similar Tₜ's in lysate conditions for varying NaCl concentrations. That is, they are thermal analogs from each of the two libraries that meet the above-described desired Tₜ characteristics for ITC purification tags.

Although previous observations suggested that decreasing the size of the ELP was likely to enhance the overall expression level of the fusion protein, it was not obvious, a priori, whether the decreased size of the tag would adversely affect purification of ELP fusion proteins by ITC. Therefore, in addition to its effect on the expression level of the target protein, the effect of the ELP tag length on the purification efficiency (i.e., degree of recovery) and on the purity of the target protein after ITC purification was explored.

The SDS-PAGE and spectrophotometry results showed that decreasing the ELP molecular weight from 36 kDa to 9 kDa enhanced expression of the fusion protein by nearly four-fold, and did not adversely affect the purity of the final protein under any of the solution conditions (i.e., NaCl concentration and temperature) used to induce the inverse transition. The level of expression with the ELP[V-20] tag was comparable to that of free thioredoxin, indicating that further reduction of the ELP tag would not be expected to increase the thioredoxin yield.

One possible explanation for the observed increase in thioredoxin yield as the ELP tag length was reduced is that, for given culture conditions, the mass of ELP that can be expressed by the cells is limited independent of ELP chain length. This was supported by the results, as well as by observations with other ELPs of various molecular weight. Such a limitation would likely be engendered by a metabolic factor, perhaps by an insufficient tRNA pool and/or by amino acid depletion due to the highly repetitious ELP sequence. If the mass yield of ELP is a limiting factor, then this provides a rationale for the increased thioredoxin yields with the ELP[V-20] tag. For a given gravimetric yield of ELP, decreasing the ELP chain length increases the molar yield of the fusion protein, and hence, the target protein. Furthermore, this also suggests that increasing the gravimetric yield of ELP, e.g., through supplementation of specific, ELP-related amino acids during culture, offers another potential route for improvement of the fusion protein yield.

Although the yield of the target protein was increased with the shorter ELP1 [V-20] tag, this benefit entailed a more complicated transition behavior. The efficiency of recovery with this tag depends on the solution conditions used for ITC, whereas, with the larger ELP1 [V₅A₂G₃-90] tag, recovery of the fusion protein was complete under all solution conditions (results not shown). Thus, although the truncated ELP1 [V-20] tag enabled thioredoxin to be purified to homogeneity by ITC, the efficiency of purification was sensitive to the specific conditions chosen to induce the inverse transition.

The turbidity and DLS data provided insights into the sensitivity of purification efficiency for the smaller ELP1 [V-20] tag on solution conditions. While solutions of thioredoxin-ELP1 [V₅A₂G₃-90] remained turbid at all temperatures above Tₜ, the turbidity profiles for thioredoxin-ELP1 [V-20], after an initial rapid rise at Tₜ, began to clear with further heating at a temperature above Tₜ. This phenomenon of clearing with increasing temperature has not been previously observed, to my knowledge, with other ELPs or ELP fusion proteins. To study this complex aggregation behavior, the sizes of the fusion protein particles were measured using dynamic light scattering as a function of temperature to determine the structural basis for the markedly different turbidity profiles of the two fusion proteins.

With increasing temperature, monomers of thioredoxin-ELP1 [V₅A₂G₃-90] went through an abrupt, discontinuous phase transition to form aggregates that persisted at all temperatures above Tₜ with a steady state Rₕ of ˜2 μm. Because the aggregates were stable above the Tₜ, the aggregated protein was able to be completely recovered by centrifugation at any temperature above its Tₜ (or at any NaCl concentration for which the Tₜ was depressed to below the solution temperature).

Although thioredoxin-ELP1 [V-20] also exhibited an abrupt phase transition to form aggregates, these aggregates were not stable at all temperatures above its phase transition. As the temperature was increased beyond the Tₜ, small aggregates with Rₕ of ˜12 nm formed at the expense of mass in the larger aggregates, which also showed a decrease in size with increasing temperature. This provides a structural rationale for the decrease in turbidity observed above the Tₜ of thioredoxin-ELP1 [V-20]. Upon heating to temperatures greater than Tₜ (beginning ˜10° C. above Tₜ for PBS with 1 M NaCl, and ˜15° C. above Tₜ for PBS with 2 M NaCl), larger scattering centers were converted to small particles that scatter light less effectively. The formation of these 12 nm particles at the expense of the larger aggregates resulted in incomplete recovery by centrifugation of the fusion protein from the soluble lysate. Thus, when ELP1 [V-20] (and potentially other small ELP tags) were used for purification of fusion proteins, it was imperative for complete protein recovery that a NaCl concentration and complimentary solution temperature be chosen such that only the larger aggregates, which are easily separable by centrifugation, were present in suspension.

On the basis of size alone, the precise structure of the 12 nm particle was not able to be predicted. However, the particle may be a micelle-like structure containing a small number of fusion protein molecules (perhaps on the order of 40 to 60) that are aggregated such that solvated thioredoxin domains encase the collapsed, hydrophobic ELP domains in the particle's core. The size of the observed particle (Rₕ≈12 nm) would be consistent with such a structure, as the hydrophilic thioredoxin “head” was ˜3 nm in diameter and the hydrophobic 20 pentamer ELP “tail” was ˜7 nm in length.

The proximity of the thioredoxin molecules required in such a micellular structure may also explain the irreversible aggregation that is observed at temperatures greater than ˜55° C. Denaturation at this low temperature was only observed for thioreoxin fused to ELP1 [V-20], and only for NaCl concentrations of 1 M and greater. And, it is only for these conditions that the 12 nm particle was observed. An extremely high effective concentration of thioredoxin in the solvated, hydrophilic shell of the micelle, with little ELP buffering between thioredoxin molecules, is consistent with the observed decrease in thermal stability.

Appropriate selection of NaCl concentration and solution temperature is appropriate to efficiently carrying out ITC. Three centrifugation temperatures were selected for experimental convenience: 12° C. when a microcentrifuge was placed in a 4° C. refrigerated laboratory cabinet, 33° C. when placed on a laboratory bench top at 22° C., and 49° C. when placed in a 37° C. static incubator (all sample temperatures were measured directly by thermocouple after a 10 minute centrifugation). The NaCl concentrations were selected in 1 M increments to depress the Tₜ to some point below each centrifugation temperature.

For the first two examples, recovery was incomplete because at these combinations of centrifugation temperature and NaCl concentration, thioredoxin-ELP1 [V-20] showed a two phase behavior where larger aggregates coexisted with the 12 nm particles. Because of their small mass, these particles remained suspended during centrifugation, and only the fraction of fusion protein contained in the larger aggregate phase was removed by centrifugation and recovered in the resolubilized pellet. At 49° C., the thioredoxin-ELP1 [V-20] turbidity profile in PBS with 1 M NaCl was significantly decreased from its maximum value, and data showed that a majority of the scattering intensity came from the 12 nm particles. Correspondingly, the SDS-PAGE data showed that only a small fraction of the fusion protein present was captured by centrifugation during ITC purification. At 33° C. in PBS with 2 M NaCl, although still below its maximum value, the turbidity of thioredoxin-ELP1 [V-20] was closer to its peak value, and the data showed that the scattering intensity attributed to the 12 nm particle was much smaller. Consistent with these observations, a majority of fusion protein was captured by ITC purification as ascertained by SDS-PAGE, although loss in the supernatant due to the 12 nm particles was still significant.

Using a centrifugation temperature of 12° C. in PBS with 3 M NaCl, recovery of the fusion protein in the resolubilized pellet was nearly complete. Under these conditions, the solution turbidity was very near its maximum value. The degree of turbidity, combined with the trends in particle size distribution established for lower salt concentrations, suggest that the complete recovery obtained by ITC with these conditions is explained by the presence of only the larger aggregates for these solution conditions.

These examples illustrate that for efficient ITC purification of thioredoxin-ELP1 [V-20], and potentially for other soluble fusion proteins with small ELP tags, the NaCl concentration and centrifugation temperature should be selected to achieve the maximum point in the turbidity profile. For microcentrifuges without temperature control, this is most practically achieved by determining the centrifuge sample temperature, and then adjusting the Tₜ of the fusion protein by the precise addition of salt. For larger centrifuges that are equipped with refrigeration systems, recovery efficiency can be maximized by the combined alteration of NaCl concentration and centrifugation temperature. The required precision in controlling solution conditions during ITC for thioredoxin-ELP1 [V-20] versus that for thioredoxin-ELP1 [V₅A₂G₃-90], which can be fully recovered using any combination of temperature and salt concentration that induces the inverse transition, is the price paid for the four-fold increase in yield of the target protein.

Decreasing the length of the ELP purification tag from 36 to 9 kDa produced a four-fold increase in the expression levels of E. coli thioredoxin, a model target protein. The expression level with the 9 kDa tag was similar to that of free thioredoxin expressed without an ELP tag, and therefore further reduction of the ELP tag size is not likely to provide any additional benefit. Although truncation of the ELP did not adversely affect the purity of the final protein product, it is important to select an appropriate combination of salt concentration and solution temperature to favor the formation of larger aggregates during ITC purification.

### Example 3

### High-Throughput Purification of Recombinant Proteins Using ELP Tags

The gene for the 5-polypentapeptide VPGVG ELP sequence was constructed by annealing two 5′-phosphorylated synthetic oligonucleotides (Integrated DNA Technologies, Coralville, Iowa) to yield double stranded DNA with PflMI and HinDIII compatible ends. This gene was inserted into a PflMI/HinDIII linearized and dephosphorylated modified pUC-19 (New England Biolabs, Beverly, Mass.) vector and polymerized using recursive directional ligation with PflMI and BglI (Meyer, 1999; Meyer, 2000) to generate the gene for the 20-polypentapeptide ELP sequence. This ELP gene was then excised with PflMI and BglI, gel purified (QIAquick Gel Extraction Kit, Qiagen, Valencia, Calif.), and inserted into a SfiI linearized and dephosphorylated modified pET32b vector (Novagen, Madison, Wis.; Meyer, 1999). This expression vector was then transformed into the BLR(DE3) (Novagen) E. Coli expression strain.

The aforementioned cells were taken from frozen (DMSO) stock and streaked onto agar plates supplanted with 100 μg/ml ampicillin and allowed to grow overnight. Two hundred microliters of growth media (100 μg/ml ampicillin in CircleGrow media; Qbiogene, Inc., Carlsbad, Calif.) were injected into each well of a standard 96 well microplate (Costar, Corning Inc., Corning, N.Y.) using a multichannel pipetter. Using 200 μl pipet tips, each well of the microplate was inoculated with a pinhead-sized aggregation of cells from colonies on the aforementioned agar plates. With the lid on, the microplate was incubated at 37° C. and shaken at 275 r.p.m. The microplate was held in place in the shaker using an ad hoc microplate holder. The cultures were induced by adding isopropyl α-thiogalactopyranoside to a final concentration of 1 mM when the OD₆₅₀ reached 0.65 for a majority of the cultures as measured using a microplate reader (Thermomax; Molecular Devices Co., Sunnyvale, Calif.)—this optical density corresponds to an OD₆₅₀ of 2.0 as measured using an UV-visible spectrophotometer (UV-1601, Shimadzu Scientific Instruments, Inc.). The cultures were incubated and shaken for 4 hours post-induction and then harvested by centrifugation at 1100 g for 40 minutes at 4° C. using matched-weight microplate carrier adaptors (Beckman Instruments, Inc., Palo Alto, Calif.). The media was discarded and the cell pellets were frozen in the microplates at −80° C. until they were ready to be purified.

The ELP1 [V-20]/thioredoxin protein was purified from cell cultures in the microplates as follows. The cells were lysed by adding 1 μl of lysozyme solution (25 mg/ml; Grade VI; Sigma, St. Louis, Mo.) and 25 ul of lysis buffer (50 mM NaCl, 5% glycerol, 50 mM Tris-HCl, pH 7.5) to each well. The micro plate was then shaken using an orbital shaker at 4° C. for 20 minutes. Two μl of 1.35% (by mass) sodium doxycholate solution were added to each well and the microplate was shaken at 4° C. for 5 minutes. Two μl of deoxyribonuclease I solution (100 units/ul; Type II; Sigma, St. Louis, Mo.) were added to each well and the microplate was shaken at 4° C. for 10 minutes. The microplate was then centrifuged at 1100 g for 20 minutes at 4° C. using matched-weight microplate carrier adaptors (Beckman Instruments, Inc., Palo Alto, Calif.) to pellet cell particulates and insoluble proteins. Two μl of 10% (by mass) polyethylenimine solution was added to each well and the microplate was shaken at 4° C. for 15 minutes. The microplate was then centrifuged at 1100 g for 20 minutes at 4° C. to pellet DNA. The supernatants were transferred to wells on a new microplate and the old microplate was discarded. To induce ELP1 [V-20]/thioredoxin aggregation, 20 μl of saturated NaCl solution was added to each well; a marked increase in turbidity indicated aggregation of the target protein. To pellet the aggregated proteins, the microplate was centrifuged at 1100 g for 40 minutes at 30° C. The protein pellets were resolubilized in 30 μl of phosphate buffer solution after which the microplate was centrifuged at 1100 g for 20 minutes at 4° C. to remove insoluble lipids. Finally, the purified protein supernatents were transferred to wells of a new microplate and stored at 4° C. SDS-PAGE gel analysis for the ELP1 [V-20]/thioredoxin fusion protein purified by ITC was carried out.

Alternatively, ELPs/ELP-fusion proteins can be purified using a commercially available extraction reagent in accordance with the following protocol. Lyse cells by adding 25 microliters of Novagen BugBuster Protein Extraction Reagent to each microplate well. The microplate is placed on a Fisher Vortex Genie at shaker speed 2 (alternatively on an orbital shaker at maximum speed) for fifteen minutes at room temperature. Using the microplate adaptors, centrifugation is conducted (2300 rpm, 1700×g for Beckman adaptor for the JS4.2 rotor) for 20 minutes at 4 degrees Celsius to form a pellet. Add 2 microliters polyethylenimine (to 0.66%) to the wells and shake using Vortex Genie or shaker for 5 minutes. Incubate on ice 10 minutes, shaking occasionally. Using the microplate adaptors, centrifuge at maximum speed for 25 minutes at 4 degrees Celsius. Transfer the supernatant to the new microplate and discard the old microplate with the pellet. Add NaCl (crystals) and/or increase the solution temperature to induce ELP aggregation. Mix by shaking only—pipeting will aggregate the ELP on the pipet tip. Solution should turn turbid to some extent. Centrifuge at a temperature above the transition temperature (2300 rpm, 1700 g, 35-40 degrees Celsius, 45 minutes). Discard supernatant and resuspend the pellet (typically non-visible or a tiny pellet) in 30 microliters of cold buffer of choice (PBS) by repeatedly pipeting around the bottom and walls of the well. Centrifuge (2300 rpm, 1700×g, 4 degrees Celsius, 20 minutes) to spin out insoluble impurities such as lipids. Transfer the supernatant to another microplate. The purified ELP may be stored frozen at −80 degrees Celsius in the microplate until ready for use. (For fusions, it must be ensured that freezing is suitable for the fusion protein.) The appropriate NaCl concentration and temperature employed in this technique depends on the ELP, fusion partner, and ELP concentration. The objective is to lower the effective ELP transition temperature at least 3 to 5 degrees below the solution temperature. An effective transition temperature of 25-30 degrees Celsius and warm centrifugation at 35-40 degrees Celsius has been usefully employed, although higher temperatures may be used if tolerated by the fusion protein.

Protein concentration was determined by measuring A₂₈₀ (UV-1601, Shimadzu Scientific Instruments, Inc.) and using the molar extinction coefficient for ELP1 [V-20]/Thioredoxin (ε=19,870); this assumes that the ELP1 [V-20]/Thioredoxin protein samples are pure of protein and DNA impurities. Thioredoxin activity was determined using an insulin reduction assay (Holmgren, 1984).

For the construction of the fusion protein, a small ELP tag was designed with a Tₜ of around 70° C., using previously published theoretical Tₜ data (Urry, 1991). Characterization of the ELP tag showed that the Tₜ was 76.2° C., confirming that it is possible to rationally design ELP tags with specified Tₜ. For the ELP/thioredoxin fusion protein, the Tₜ in low salt buffer, 1 M, and 2 M salt solutions were 68° C., 37° C. and 18° C., respectively, confirming that fusion of a soluble protein to an ELP tag minimally affects its Tₜ and showing that the Tₜ can be manipulated over a wide range by adjusting the salt concentration.

Based on the foregoing, the creation of a family of plasmid expression vectors that contain an ELP sequence and a polylinker region (into which the target protein is inserted) joined by a cleavage site can be employed to facilitate the expression of a variety of proteins. The ELP sequences embedded in such family of plasmids can have different transition temperatures (by varying the identity of the guest residue). The expression vector for a particular target protein is desirably selected based on the protein's surface hydrophobicity characteristics. The salt concentration of the solution then is adjusted during purification to obtain the desired T.

For protein expression involving growth of cell cultures in microplate wells, the cell cultures can be desirably induced at OD₆₀₀≈2 and grown for 4 hours post-induction. The cell density at induction for the microplate growths is two to three times that achieved by conventional protein expression protocols. Even at these high cell densities, rapid and healthy cell growth can be maintained in the microplate wells by aeration of the cultures, which as grown in the wells are characterized by a high surface area to volume ratio. Cell cultures that are grown longer post-induction yielded minimally more target protein, and growth using a hyper expression protocol (Guda, 1995) had much more contaminant protein (around tenfold) with minimally more fusion protein. In order to avoid evaporation of the cell media in the high surface area to volume ratio cell growth in the microplate wells, it was necessary to cover the microplate with an appropriate lid during growth and to infuse the cell growth with additional media during induction. On a per liter basis, cultures grown in the microplate wells had a higher level of fusion protein expression than cultures grown with conventional protocols.

High throughput protein purification utilizing ITC was successful when cells were lysed with commercial nonionic protein extraction formulations. After cell lysis, addition of polyethylenimine removed nucleic acids and high molecular mass proteins from the soluble fraction of the crude lysate upon centrifugation. At the fusion protein and salt concentrations of the soluble lysate, the Tₜ of the fusion protein was approximately 65° C. Heating the soluble lysate above this temperature to induce fusion protein aggregation denatures and precipitates soluble contaminant proteins as well as the target protein itself. Furthermore, this temperature could not be maintained within the centrifuge chamber during centrifugation. Therefore, salt was added to the soluble lysate to approximately 2 M; this depressed the Tₜ of the fusion protein to approximately 18° C., allowing for aggregation of the fusion protein at room temperature. This salt concentration did not precipitate any contaminant proteins nor did it alter the functionality of the final purified protein product.

High throughput protein purification using ITC was both effective and efficient. About 15% of the expressed fusion protein was lost in the insoluble protein fraction of the cell lysate. Centrifugation of the sample after fusion protein aggregation effectively separated the proteins: 90% of the fusion protein was pelleted while 10% of the fusion protein remained in the supernatant along with all soluble contaminant proteins. Overall, about 75% of the expressed protein was abstracted using ITC purification and E. coli contaminant protein levels in the purified products were below those detectable by SDS-PAGE. The purification process can be expedited and purification efficiency increased by increasing the centrifugation speeds; higher centrifugation speeds allow for reduced centrifugation times and at higher centrifugation speeds (5000 g), all of the fusion protein is pelleted during centrifugation post aggregation. Addition of thrombin completely cleaved the fusion protein and a second round of ITC separated the ELP tag from the thioredoxin target protein with no loss of thioredoxin.

The average amount of fusion protein purified per well determined using absorbance measurements (A₂₈₀, ε=19,870) was 33 ug with a standard deviation of 8.5 ug. Values were dispersed evenly between 19.7 and 48.3 ug per well. The large variation in yield of purified protein was due more to the different amounts of protein expressed in the different wells than to a variation in the purification efficiency of the ITC process. Varying amounts of protein were expressed in the different cell cultures because 1) the imprecision of the inoculation meant that cell cultures had varying amounts of cells to begin with and 2) due in all likelihood to more abundant aeration, the cell cultures in peripheral wells tended to have faster growth and reach a higher stationary phase cell density. For simplicity of effort, all of the cell cultures were induced and then harvested at the same times as opposed to induction and harvesting of individual cell cultures.

The enzymatic activity of the thioredoxin target protein was measured using an insulin reduction assay. The average amount of fusion protein per well, determined on the basis of such enzymatic activity, was 35.7 ug with a standard deviation of 8.0 ug. Again, values were dispersed evenly, between a minimum of 24.6 and a maximum of 50.8 ug per well. It is important to note that thioredoxin was enzymatically active though still attached to the ELP tag. The thioredoxin expressed and purified using this high throughput ITC technique had, on average, 10.3% greater enzymatic activity per unit mass than that of commercial thioredoxin (Sigma), a testament to the gentleness of and purity achieved by the ITC process.

On average, high throughput ELP/thioredoxin protein expression and purification produced around 160 mg of protein per liter of growth. This is comparable to ELP/thioredoxin yields obtained using conventional protein expression and ITC purification methods (140-200 mg protein/L of growth).

An SDS-PAGE gel of the stages of high throughput protein purification using microplates and inverse transition cycling was carried out according to the above-described procedure, in which ELP/thioredoxin fusion protein was purified using the documented protocol. Gel samples were denatured with SDS, reduced with beta-mercaptoethanol, and run at 200 V for 45 minutes on a 10-20% gradient Tris-HCl gel.

Histograms were employed for quantitization of purified protein samples, including a histogram of total fusion protein per well determined using absorbance measurements (A₂₈₀, ε=19,870) (n=20, μ=32.97, σ=8.48) and a histogram of fusion protein functionality/purity for each sample compared to commercial thioredoxin (from Sigma) (n=20, μ=110.37%, σ=16.54%).

Considering such high throughput protein expression and purification method, it is noted that whereas nickel-chelated multiwell plates can purify only 1 ng of His-tagged protein per well, the capacity of high throughput purification using ITC is limited only by the amount of the protein that can expressed by cultures grown in the well; for ELP tagged proteins, the level of protein expression is in the tens of microgram range.

High throughput purification using ITC thus provides high yields, producing sufficient fusion protein for purification of the peptide active therapeutic agent-ELP construct to produce active ingredient for therapeutic compositions. Milligram levels of purified fusion protein can be obtained by growing cell cultures in other vessels and transferring the resuspended cell pellet to the multiwell plate for the purification process. Finally, such high throughput purification technique is technically simpler and less expensive than current conventional commercial high throughput purification methods as it requires only one transfer of purification intermediates to a new multiwell plate.

### Example 4

### Construction of Various ELP Gene Expression Series

### Bacterial Strains and Plasmids

Cloning steps were conducted in Escherichia coli strain XL1-Blue (recA1, endA1, gyrA96, thi-1, hsdR17 (rₖ⁻, mₖ⁺), supE44, relA1, lac[F′, proAB, lacI^{q}ZΔM15, Tn10 (Tet^{r})] (Stratagene La Jolla, Calif.). pUC19 (NEB, Beverly, Mass.) was used as the cloning vector the ELP construction (Meyer and Chilkoti, 1999). Modified forms of pET15b and pET24d vectors (Novagen) were used to express ELP and ELP-fusion proteins in BL21 Star (DE3) strain (F⁻, ompT, hsdS_{B} (r_{B}⁻m_{B}⁻), gal, dcm, rne131, (DE3)) (Invitrogen Carlsbed, Calif.) or BLR(DE3) (F⁻, ompT, hsdS_{B} (r_{B}⁻m_{B}⁻), gal, dcm, Δ(srl-recA) 306::Tn10(Tc^{R})(DE3)) (Novagen Madison, Wis.). Synthetic DNA oligos were purchased from Integrated DNA Technologies, Coralville, Iowa. All vector constructs were made using standard molecular biology protocols (Ausubel, et al., 1995).

### Construction of ELP1 [V₅A₂G₃] Gene Series

The ELP1 [V₅A₂G₃] series designate polypeptides containing multiple repeating units of the pentapeptide VPGXG, where X is valine, alanine, and glycine at a relative ratio of 5:2:3.

The ELP1 [V₅A₂G₃] series monomer, ELP1 [V₅A₂G₃-10], was created by annealing four 5′ phosphorylated, PAGE purified synthetic oligos to form double stranded DNA with EcoR1 and HindIII compatible ends (Meyer and Chilkoti, 1999). The oligos were annealed in a 1 μM mixture of the four oligos in 50 μl 1× ligase buffer (Invitrogen) to 95° C. in a heating block than the block was allowed to cool slowly to room temperature. The ELP1 [V₅A₂G₃-10]/EcoRI-HindIII DNA segment was ligated into a pUC19 vector digested with EcoR1 and HindIII and CIAP dephosphorylated (Invitrogen) to form pUC19-ELP1[V₅A₂G₃-10]. Building of the ELP1 [V₅A₂G₃] series library began by inserting ELP1 [V₅A₂G₃-10] PflM1/Bgl1 fragment from pUC19-ELP1 [V₅A₂G₃-10] into pUC19-ELP1[V₅A₂G₃-10] linearized with PflM1 and dephosphorylated with CIAP to create pUC19-ELP1 [V₅A₂G₃-20]. pUC19-ELP1[V₅A₂G₃-20] was then built up to pUC19-ELP1[V₅A₂G₃-30] and pUC19-ELP1[V₅A₂G₃-40] by ligating ELP1[V₅A₂G₃-10] or ELP1[V₅A₂G₃-20] PflM1/Bgl1 fragments respectively into PflM1 digested pUC19-ELP1 [V₅A₂G₃-20]. This procedure was used to expand the ELP1 [V₅A₂G₃] series to create pUC19-ELP1[V₅A₂G₃-60], pUC19-ELP1[V₅A₂G₃-90] and pUC19-ELP1[V₅A₂G₃-180] genes.

### Construction of ELP1 [K₁V₂F₁] Gene Series

The ELP1 [K₁V₂F₁] series designate polypeptides containing multiple repeating units of the pentapeptide VPGXG, where X is lysine, valine, and phenylalanine at a relative ratio of 1:2:1.

The ELP1 [K₁V₂F₁] series monomer, ELP1 [K₁V₂F₁-4] (SEQ ID NO: 18), was created by annealing two 5′ phosphorylated, PAGE purified synthetic oligos to form double stranded DNA with EcoR1 and HindIII compatible ends (Meyer and Chilkoti, 1999). The oligos were annealed in a 1 μM mixture of the four oligos in 50 μl 1× ligase buffer (Invitrogen) to 95° C. in a heating block than the block was allowed to cool slowly to room temperature. The ELP1 [K₁V₂F₁-4]/EcoR1-HindIII DNA segment was ligated into a pUC19 vector digested with EcoR1 and HindIII and CIAP dephosphorylated (Invitrogen) to form pUC19-ELP1[K₁V₂F₁-4]. Building of the ELP1 [K_{I}V₂Fᵢ] series library began by inserting ELP1 [K₁V₂F₁-4] PflM1/Bgl1 fragment from pUC19-ELP1[K₁V₂F₁-4] into pUC19-ELP1[K₁V₂F₁-4] linearized with PflM1 and dephosphorylated with CIAP to create pUC19-ELP1[K₁V₂F₁-8]. Using the same procedure the ELP1 [K₁V₂F₁] series was doubled at each ligation to form pUC19-ELP1[K₁V₂F₁-16], pUC19-ELP1[K₁V₂F₁-32], pUC19-ELP1[K₁V₂F₁-64] and pUC19-ELP1[K₁V₂F₁-128].

### Construction of ELP1 [K₁V₇F₁] Gene Series

The ELP1 [K₁V₇F₁] series designate polypeptides containing multiple repeating units of the pentapeptide VPGXG, where X is lysine, valine, and phenylalanine at a relative ratio of 1:7:1.

The ELP1 [K₁V₇F₁] series monomer, ELP1 [K₁V₇F₁-9] (SEQ ID NO: 19), was created by annealing four 5′ phosphorylated, PAGE purified synthetic oligos to form double stranded DNA with PflMI and HindIII compatible ends. The ELP1 [K₁V₇F₁-9] DNA segment was than ligated into PflMI/HindIII dephosphorylated pUC19-ELP1 [V₅A₂G₃-180] vector thereby substituting ELP1 [V₅A₂G₃-180] for ELP1 [K₁V₇F₁-9] to create the pUC19-ELP1 [K₁V₇F₁-9] monomer. The ELP1 [K₁V₇F₁] series was expanded in the same manor as the ELP1 [K₁V₂F₁] series to create pUC19-ELP1[K₁V₇F₁-18], pUC19-ELP1 [K₁V₇F₁-36], pUC19-ELP1[K₁V₇F₁-72] and pUC19-ELP1[K₁V₇F₁-144].

### Construction of ELP1 [V] Gene Series

The ELP1 [V] series designate polypeptides containing multiple repeating units of the pentapeptide VPGXG, where X is exclusively valine.

The ELP1 [V] series monomer, ELP1 [V-5] (SEQ ID NO: 14), was created by annealing two 5′ phosphorylated, PAGE purified synthetic oligos to form double stranded DNA with EcoRI and HindIII compatible ends. The ELP1 [V-5] DNA segment was than ligated into EcoRI/HindIII dephosphorylated pUC19 vector to create the pUC19-ELP1 [V-5] monomer. The ELP1 [V] series was created in the same manor as the ELP1 [V₅A₂G₃] series, ultimately expanding pUC19-ELP1 [V-5] to pUC19-ELP1 [V-60] and pUC19-ELP1 [V-120].

### Construction of ELP2 Gene Series

The ELP2 series designate polypeptides containing multiple repeating units of the pentapeptide AVGVP.

The ELP2 series monomer, ELP2 [5] (SEQ ID NO: 20), was created by annealing two 5′ phosphorylated, PAGE purified synthetic oligos to form double stranded DNA with EcoRI and HindIII compatible ends. The ELP2 [5] DNA segment was than ligated into EcoRI/HindIII dephosphorylated pUC19 vector to create the pUC19-ELP2[5] monomer. The ELP2 series was expanded in the same manor as the ELP1 [K₁V₂F₁] series to create pUC19-ELP2[10], pUC19-ELP2[30], pUC19-ELP2[60] and pUC19-ELP2[120].

### Construction of ELP3 [V] Gene Series

The ELP3 [V] series designate polypeptides containing multiple repeating units of the pentapeptide IPGXG, where X is exclusively valine.

The ELP3 [V] series monomer, ELP3 [V-5] (SEQ ID NO: 21), was created by annealing two 5′ phosphorylated, PAGE purified synthetic oligos to form double stranded DNA with PfLM1 amino terminal and GGC carboxyl terminal compatible ends due to the lack of a convenient carboxyl terminal restriction site but still enable seamless addition of the monomer. The ELP3 [V-5] DNA segment was then ligated into PflM1/BglI dephosphorylated pUC19-ELP4[V-5], thereby substituting ELP4 [V-5] for ELP3 [V-5] to create the pUC19-ELP3[V-5] monomer. The ELP3 [V] series was expanded by ligating the annealed ELP3 oligos into pUC19-ELP3[V-5] digested with PflM1. Each ligation expands the ELP3 [V] series by 5 to create ELP3 [V-10], ELP3 [V-15], etc.

### Construction of the ELP4 [V] Gene Series

The ELP4 [V] series designate polypeptides containing multiple repeating units of the pentapeptide LPGXG, where X is exclusively valine.

The ELP4 [V] series monomer, ELP4 [V-5] (SEQ ID NO: 22), was created by annealing two 5′ phosphorylated, PAGE purified synthetic oligos to form double stranded DNA with EcoRI and HindIII compatible ends. The ELP4 [V-5] DNA segment was than ligated into EcoRI/HindIII dephosphorylated pUC19 vector to create the pUC19-ELP4[V-5] monomer. The ELP4 [V] series was expanded in the same manor as the ELP1 [K₁V₂Fᵢ] series to create pUC19-ELP4[V-10], pUC19-ELP4[V-30], pUC19-ELP4[V-60] and pUC19-ELP4[V-120].

The ELP genes were also inserted into other vectors such as pET15b-SD0, pET15b-SD3, pET15b-SD5, pET15b-SD6, and pET24d-SD21. The pET vector series are available from Novagen, San Diego, Calif.

The pET15b-SD0 vector was formed by modifying the pET15b vector using SD0 double-stranded DNA segment containing the multicloning restriction site (Sac1-Nde1-Nco1-Xho1-SnaB1-BamH1). The SD0 double-stranded DNA segment had Xba1 and BamH1 compatible ends and was ligated into Xba1/BamH1 linearized and 5′-dephosphorylated pET15b to form the pet15b-SD0 vector.

The pET15b-SD3 vector was formed by modifying the pET15b-SD0 vector using SD3 double-stranded DNA segment containing a SfiI restriction site upstream of a hinge region-thrombin cleavage site followed by the multicloning site (Nde1-Nco1-Xho1-SnaB1-BamHI). The SD3 double-stranded DNA segment had Sac1 and Nde1 compatible ends and was ligated into Sac1/Nde1 linearized and 5′-dephosphorylated pET15b-SD0 to form the pET15b-SD3 vector.

The pET15b-SD5 vector was formed by modifying the pET15b-SD3 vector using the SD5 double-stranded DNA segment containing a Sfi1 restriction site upstream of a thrombin cleavage site followed by a hinge and the multicloning site (Nde1-Nco1-Xho1-SnaB1-BamHI). The SD5 double-stranded DNA segment had Sfi1 and Nde1 compatible ends and was ligated into Sfi1/Nde1 linearized and 5′-dephosphorylated pET15b-SD3 to form the pET15b-SD5 vector.

The pET15b-SD6 vector was formed by modifying the pET15b-SD3 vector using the SD6 double-stranded DNA segment containing a Sfi1 restriction site upstream of a linker region-TEV cleavage site followed by the multicloning site (Nde1-Nco1-Xho1-SnaB1-BamHI). The SD6 double-stranded DNA segment had Sfi1 and Nde1 compatible ends and was ligated into Sfi1/Nde1 linearized and 5′-dephosphorylated pET15b-SD3 to form the pET15b-SD6 vector.

The pET24d-SD21 vector was formed by modifying the pET24d vector using the SD21 double-stranded DNA segment with Nco1 and Nhe1 compatible ends. The SD21 double-stranded DNA segment was ligated into Nco1/Nhe1 linearized and 5′ dephosphorylated pET24d to create the pET24d-SD21 vector, which contained a new multi-cloning site NcoI-SfiI-NheI-BamHI-EcoR1-SacI-SalI-HindIII-NotI-XhoI with two stop codons directly after the SfiI site for insertion and expression of ELP with the minimum number of extra amino acids.

The pUC19-ELP1 [V₅A₂G₃-60], pUC19-ELP1[V₅A₂G₃-90], and pUC19-ELP1[V₅A₂G₃-180] plasmids produced in XL1-Blue were digested with PflM1 and Bgl1, and the ELP-containing fragments were ligated into the Sfi1 site of the pET15b-SD3 expression vector as described hereinabove to create pET15b-SD3-ELP1[V₅A₂G₃-60], pET15b-SD5-ELP1[V₅A₂G₃-90] and pET15b-SD5-ELP1[V₅A₂G₃-180], respectively.

The pUC19-ELP1[V₅A₂G₃-90], pUC19-ELP1 [V₅A₂G₃-180], pUC19-ELP1[V-60] and pUC19-ELP1 [V-120] plasmids produced in XL1-Blue were digested with PflM1 and Bgl1, and the ELP-containing fragments were ligated into the Sfi1 site of the pET15b-SD5 expression vector as described hereinabove to create pET15b-SD5-ELP1[V₅A₂G₃-90], pET15b-SD5-ELP1[V₅A₂G₃-180], pET15b-SD5-ELP1[V-60] and pET15b-SD5-ELP1[V-120], respectively.

The pUC19-ELP1 [V₅A₂G₃-90] plasmid produced in XL1-Blue was digested with PflM1 and Bgl1, and the ELP-containing fragment was ligated into the Sfi1 site of the pET15b-SD6 expression vector as described hereinabove to create pET15b-SD6-ELP1[V₅A₂G₃-90].

The pUC19-ELP1 [K₁V₂F₁-64], and pUC19-ELP1 [K₁V₂F₁-128] plasmids produced in XL1-Blue were digested with PflM1 and Bgl1, and the ELP-containing fragments were ligated into the Sfi1 site of the pET24d-SD21 expression vector as described hereinabove to create pET24d-SD21-ELP1[K₁V₂F₁-64] and pET24d-SD21-ELP1 [K₁V₂F₁-128], respectively.

The pUC19-ELP1[K₁V₇F₁-72] and pUC19-ELP1[K₁V₇F₁-144] plasmids produced in XL1-Blue were digested with PflM1 and Bgl1, and the ELP-containing fragments were ligated into the Sfi1 site of the pET24d-SD21 expression vector as described hereinabove to create pET24d-SD21-ELP1 [K₁V₇F₁-72] pET24d-SD21-ELP1 [K_{I}V₇F₁-144], respectively.

The pUC19-ELP2[60] and pUC19-ELP2[120] plasmids produced in XL1-Blue were digested with NcoI and HindIII, and the ELP-containing fragments were ligated into the NcoI and HindIII sites of the pET24d-SD21 expression vector as described hereinabove to create pET24d-SD21-ELP2[60], pET24d-SD21-ELP2[120], respectively.

The pUC19-ELP4[V-60] and pUC19-ELP4[V-120] plasmids produced in XL1-Blue were digested with NcoI and HindIII, and the ELP-containing fragments were ligated into the NcoI and HindIII sites of the pET24d-SD21 expression vector as described hereinabove to create pET24d-SD21-ELP4[V-60], pET24d-SD21-ELP4[V-120], respectively.

### Example 5

### Construction, Isolation and Purification of Various Fusion Proteins

It is to be noted that the following fusion proteins illustrate a variety of peptide active therapeutic agent and ELP species in specific combinations.

Although these fusion proteins were designed with cleavage sites between the respective peptide active therapeutic agent and ELP moieties, for use in cleaving reactions to produce peptide active therapeutic agent and ELP moieties for further study, corresponding peptide active therapeutic agent-ELP constructs lacking such cleavage sites are readily produced, by the simple expedient of direct bonding of the peptide active therapeutic agent to the ELP, without any interposed cleavage group or moiety that is susceptible to scission by proteases or other degradative agents or conditions that may be encountered by the construct in vivo subsequent to its administration.

Experiments were conducted to show the use of various target proteins (peptide active therapeutic agents) in forming ELP-containing fusion proteins and the inverse phase transition behavior exhibited by such fusion proteins. Specifically, the following thirty-six (36) ELP-containing fusion proteins were formed in E. coli by using known recombinant expression techniques consistent with the teachings and disclosures hereinabove:
Insulin A peptide and ELP1 [V-60] polypeptide with an enterokinase protease cleavage site therebetween (SEQ ID NO: 23);
Insulin A peptide and ELP1 [V₅A₂G₃-90] polypeptide with an enterokinase protease cleavage site therebetween (SEQ ID NO: 24);
Insulin A peptide and ELP1 [V-120] polypeptide with an enterokinase protease cleavage site therebetween (SEQ ID NO: 25);
Insulin A peptide and ELP1 [V₅A₂G₃-180] polypeptide with an enterokinase protease cleavage site therebetween (SEQ ID NO: 26);
T20 peptide and ELP1 [V-60] polypeptide with an enterokinase protease cleavage site therebetween (SEQ ID NO: 27);
T20 peptide and ELP1 [V₅A₂G₃-90] polypeptide with an enterokinase protease cleavage site therebetween (SEQ ID NO: 28);
T20 peptide and ELP1 [V-120] polypeptide with an enterokinase protease cleavage site therebetween (SEQ ID NO: 29);
T20 peptide and ELP1 [V-60] polypeptide with a thrombin protease cleavage site therebetween (SEQ ID NO: 30);
T20 peptide and ELP1 [V₅A₂G₃-90] polypeptide with a thrombin protease cleavage site therebetween (SEQ ID NO: 31);
T20 peptide and ELP1 [V-120] polypeptide with a thrombin protease cleavage site therebetween (SEQ ID NO: 32);
T20 peptide and ELP1 [V-60] polypeptide with a tobacco etch virus (TEV) protease cleavage site (cleavage between QS residues) therebetween (SEQ ID NO: 33);
T20 peptide and ELP1 [V₅A₂G₃-90] polypeptide with a TEV protease cleavage site (cleavage between QS residues) therebetween (SEQ ID NO: 34);
T20 peptide and ELP1 [V-120] polypeptide with a TEV protease cleavage site (cleavage between QS residues) therebetween (SEQ ID NO: 35);
T20 peptide and ELP1 [V-60] polypeptide with a TEV protease cleavage site (cleavage between QY residues) therebetween (SEQ ID NO: 36);
T20 peptide and ELP1 [V₅A₂G₃-90] polypeptide with a TEV protease cleavage site (cleavage between QY residues) therebetween (SEQ ID NO: 37);
T20 peptide and ELP1 [V-120] polypeptide with a TEV protease cleavage site (cleavage between QY residues) therebetween (SEQ ID NO: 38);
Interferon alpha 2B protein and ELP1 [V₅A₂G₃-90] polypeptide with a thrombin protease cleavage site therebetween (SEQ ID NO: 39);
Tobacco etch virus protease and ELP1 [V-60] polypeptide with a thrombin protease cleavage site therebetween (SEQ ID NO: 40);
Tobacco etch virus protease and ELP1 [V₅A₂G₃-90] polypeptide with a thrombin protease cleavage site therebetween (SEQ ID NO: 41);
Tobacco etch virus protease and ELP1 [V-120] polypeptide with a thrombin protease cleavage site therebetween (SEQ ID NO: 42);
Tobacco etch virus protease and ELP1 [V₅A₂G₃-180] polypeptide with a thrombin protease cleavage site therebetween (SEQ ID NO: 43);
Small heterodimer partner orphan receptor and ELP1 [V₅A₂G₃-90] polypeptide with a thrombin protease cleavage site therebetween (SEQ ID NO: 44);
Androgen receptor ligand binding domain and ELP1 [V₅A₂G₃-90] polypeptide with a thrombin protease cleavage site therebetween (SEQ ID NO: 45);
Androgen receptor ligand binding domain and ELP1 [V₅A₂G₃-180] polypeptide with a thrombin protease cleavage site therebetween (SEQ ID NO: 46);
Glucocorticoid receptor ligand binding domain and ELP1 [V₅A₂G₃-90] polypeptide with a thrombin protease cleavage site therebetween (SEQ ID NO: 47);
Estrogen receptor ligand binding domain and ELP1 [V₅A₂G₃-60] polypeptide with a thrombin protease cleavage site therebetween (SEQ ID NO: 48);
Estrogen receptor ligand binding domain and ELP1 [V₅A₂G₃-90] polypeptide with a thrombin protease cleavage site therebetween (SEQ ID NO: 49);
Estrogen receptor ligand binding domain and ELP1 [V₅A₂G₃-180] polypeptide with a thrombin protease cleavage site therebetween (SEQ ID NO: 50);
Estrogen receptor ligand binding domain and ELP1 [V₅A₂G₃-90] polypeptide with a TEV protease cleavage site (cleavage between QG residues) therebetween (SEQ ID NO: 51);
G protein alpha Q and ELP1 [V₅A₂G₃-90] polypeptide with a thrombin protease cleavage site therebetween (SEQ ID NO: 52);
G protein alpha Q and ELP1 [V₅A₂G₃-180] polypeptide with a thrombin protease cleavage site therebetween (SEQ ID NO: 53);
1-Deoxy-D-Xylulose 5-Phosphate reductoisomerase peptide and ELP1 [V₅A₂G₃-60] polypeptide with a thrombin protease cleavage site therebetween (SEQ ID NO: 54);
1-Deoxy-D-Xylulose 5-Phosphate reductoisomerase peptide and ELP1 [V₅A₂G₃-90] polypeptide with a thrombin protease cleavage site therebetween (SEQ ID NO: 55);
1-Deoxy-D-Xylulose 5-Phosphate reductoisomerase peptide and ELP1 [V₅A₂G₃-180] polypeptide with a thrombin protease cleavage site therebetween (SEQ ID NO: 56);
1-Deoxy-D-Xylulose 5-Phosphate reductoisomerase peptide and ELP1 [V₅A₂G₃-90] polypeptide with a TEV protease cleavage site (cleavage between QG residues) therebetween (SEQ ID NO: 57); and
G protein alpha S and ELP1 [V₅A₂G₃-90] polypeptide with a thrombin protease cleavage site therebetween (SEQ ID NO: 58).

All of the above-listed thirty-six ELP-containing fusion proteins were found to retain the inverse phase transition behavior of the corresponding ELP tags, and were successfully isolated and purified by using inverse transition cycling (ITC) techniques, according to the following experimental procedure:

### Isolation and Purification of Fusion Proteins Containing Insulin A Peptide (InsA)

A single colony of E. coli strain BLR (DE3) (Novagen) containing the respective ELP-InsA fusion protein was inoculated into 5 ml CircleGrow (Q-BIOgene, San Diego, Calif.) supplemented with 100 μg/ml ampicillin (Sigma) and grown at 37° C. with shaking at 250 rpm for 5 hours. The 5 ml culture was then inoculated into a 500 ml culture and allowed to grow at 25° C. for 16 hours before inducing with 1 mM IPTG for 4 hours at 25° C. The culture was harvested and suspended in 40 ml 20 mM Tris-HCL pH 7.4, 50 mM NaCl, 1 mM DTT and 1 Complete EDTA free Protease inhibitor pellet (Roche, Indianapolis, Ind.). Cells were lysed by ultrasonic disruption on ice for 3 minutes, which consisted of 10 seconds bursts at 35% power separated by 30 second cooling down intervals. Cell debris was removed by centrifugation at 20,000 g, 4° C. for 30 minutes.

Inverse phase transition was induced by adding NaCl to the cell lysate at room temperature to achieve a final concentration of 1.0 M therein, followed by centrifugation at 20,000 g for 15 minutes at room temperature. The resulting pellet contained the respective ELP-InsA fusion protein and non-specifically NaCl precipitated proteins.

The pellet was re-suspended in 40 ml ice-cold ml 20 mM Tris-HCL pH 7.4, 50 mM NaCl, 1 mM DTT and re-centrifuged at 20,000 g, 4° C. for 15 minutes to remove the non-specifically NaCl precipitated proteins. The inverse transition cycle was repeated two additional times to increase the purity of the respective ELP-InsA fusion protein and reduce the final volume to 0.5 ml.

### Isolation and Purification of Fusion Proteins Containing T20 Peptide (T20)

A single colony of E. coli strain BLR (DE3) (Novagen) containing the respective ELP-T20 fusion protein was inoculated into 500 ml CircleGrow (Q-BIOgene, San Diego, Calif.) supplemented with 100 μg/ml ampicillin (Sigma) and grown at 37° C. with shaking at 250 rpm for 24 hours. The culture was harvested and suspended in 40 ml 50 mM Tris pH 8.0, 0.5 mM EDTA and 1 Complete Protease inhibitor pellet (Roche, Indianapolis, Ind.). Cells were lysed by ultrasonic disruption on ice for 3 minutes, which consisted of 10 seconds bursts at 35% power separated by 30 second cooling down intervals. Cell debris was removed by centrifugation at 20,000 g, 4° C. for 30 minutes.

Inverse phase transition was induced by adding NaCl to the cell lysate at room temperature to achieve a final concentration of 1.0 M therein, followed by centrifugation at 20,000 g for 15 minutes at room temperature. The resulting pellet contained the respective ELP-T20 fusion protein and non-specifically NaCl precipitated proteins.

The pellet was re-suspended in 40 ml ice-cold ml 50 mM Tris pH 8.0, 0.5 mM EDTA and re-centrifuged at 20,000 g, 4° C. for 15 minutes to remove the non-specifically NaCl precipitated proteins. The inverse transition cycle was repeated two additional times to increase the purity of the respective ELP-T20 fusion protein and reduce the final volume to 5 ml.

### Isolation and Purification of Fusion Protein Containing Interferon Alpha 2B Peptide (IFNA2)

A single colony of E. coli strain BL21(DE3) TrxB⁻ (Novagen) containing the ELP-IFNA2 fusion protein and Codon Plus-RIL plasmid (Stratagene) was inoculated into 500 ml CircleGrow (Q-BIOgene, San Diego, Calif.) supplemented with 100 μg/ml ampicillin (Sigma), 25 ug/ml Chloramphenicol (Sigma) and incubated at 27° C. with shaking at 250 rpm for 48 hours. The culture was harvested and suspended in 50 mM Tris-HCL pH 7.4, 50 mM NaCl and 1 Complete EDTA free Protease inhibitor pellet (Roche, Indianapolis, Ind.). Cells were lysed by ultrasonic disruption on ice for 3 minutes, which consists of 10 seconds bursts at 35% power separated by 30 second cooling down intervals. Cell debris was removed by centrifugation at 20,000 g, 4° C. for 30 minutes.

Inverse phase transition was induced by adding NaCl to the cell lysate at room temperature to achieve a final concentration of 1.5 M, followed by centrifugation at 20,000 g for 15 minutes at room temperature. The resulting pellet contained the ELP-IFNA2 fusion protein and non-specifically NaCl precipitated proteins.

The pellet was re-suspended in 40 ml ice-cold 50 mM Tris-HCL pH 7.4 and 50 mM NaCl and re-centrifuged at 20,000 g, 4° C. for 15 minutes to remove the non-specifically NaCl precipitated proteins. The inverse transition cycle was repeated two additional times to increase the purity of the ELP-IFNA2 fusion protein and reduce the final volume to 5 ml.

### Isolation and Purification of Fusion Proteins Containing Tobacco Etch Virus Protease (TEV)

A single colony of E. coli strain BL21 star or BRL(DE3) containing pET15b-SD5-ELP-TEV constructs and Codon Plus-RIL plasmid (Stratagene) was inoculated into 500 ml CircleGrow (Q-BIOgene, San Diego, Calif.) supplemented with 100 μg/ml ampicillin (Sigma), 25 ug/ml Chloramphenicol (Sigma) and incubated at 27° C. with shaking at 250 rpm for 48 hours. The culture was harvested and suspended in 50 mM Tris-HCL pH 8.0, 1 mM EDTA, 5 mM DTT, 10% glycerol and 1 mM PMSF. Cells were lysed by ultrasonic disruption on ice for 3 minutes, consisting of 10 seconds bursts at 35% power separated by 30 second cooling down intervals. Cell debris was removed by centrifugation at 20,000 g, 4° C. for 30 minutes.

Inverse phase transition was induced by adding NaCl to the cell lysate at room temperature to achieve a final concentration of 1.5 M, followed by centrifugation at 20,000 g for 15 minutes at room temperature. The resulting pellet contained the respective ELP-TEV fusion protein and non-specifically NaCl precipitated proteins.

The pellet was re-suspended in 40 ml ice-cold 50 mM Tris-HCL pH 8.0, 1 mM EDTA, 5 mM DTT, 10% glycerol and re-centrifuged at 20,000 g, 4° C. for 15 minutes to remove the non-specifically NaCl precipitated proteins. The inverse transition cycle was repeated two additional times to increase the purity of the respective ELP-TEV fusion protein and reduce the final volume to 1 ml.

### Isolation and Purification of Fusion Protein Containing Small Heterodimer Partner Orphan Receptor (SHP)

A single colony of E. coli strain BL21 Star (DE3) containing the ELP-SHP fusion protein was inoculated into 500 ml CircleGrow (Q-BIOgene, San Diego, Calif.) supplemented with 100 mg/ml ampicillin (Sigma) and 10% sucrose and grown at 27° C. with shaking at 250 rpm for 48 hours. The culture was harvested and suspended in 50 mM Tris-HCL pH 8.0, 150 mM KCL, 1 mM DTT 1 mM EDTA and 1 Complete EDTA free Protease inhibitor pellet (Roche, Indianapolis, Ind.). Cells were lysed by ultrasonic disruption on ice for 3 minutes, which consists of 10 seconds bursts at 35% power separated by 30 second cooling down intervals. DNA and RNA in the soluble lysate were further degraded by adding 2 μl Benzonase (Novagen) and incubating at 4° C. for 30 minutes. Cell debris was removed by centrifugation at 20,000 g, 4° C. for 30 minutes.

Inverse phase transition was induced by adding NaCl to the cell lysate at room temperature to achieve a final concentration of 1.5 M, followed by centrifugation at 20,000 g for 15 minutes at room temperature. The resulting pellet contained the ELP-SHP fusion protein and non-specifically NaCl precipitated proteins.

The pellet was re-suspended in 40 ml ice-cold 50 mM Tris-HCL pH 8.0, 150 mM KCL, 1 mM DTT 1 mM EDTA, and 1% N-Octylglucoside and re-centrifuged at 20,000 g, 4° C. for 15 minutes to remove non-specific insoluble proteins. The temperature transition cycle was repeated two additional times to increase the purity of the ELP-SHP fusion protein and reduce the final volume to 2 ml.

### Isolation and Purification of Fusion Proteins Containing Androgen Receptor Ligand Binding Domain (AR-LBD)

A single colony of E. coli strain BL21 Star (DE3) containing the respective ELP-AR-LBD fusion protein was inoculated into 500 ml CircleGrow (Q-BIOgene, San Diego, Calif.) supplemented with 100 μg/ml ampicillin (Sigma) and 10 μM DHT and grown at 27° C. with shaking at 250 rpm for 48 hours. The culture was harvested and suspended in 40 ml 50 mM Hepes pH 7.5, 150 mM NaCl, 0.1% N-Octylglycoside, 10% glycerol, 1 mM DTT, 1 μM DHT and 1 Complete EDTA free Protease inhibitor pellet (Roche, Indianapolis, Ind.). Cells were lysed by ultrasonic disruption on ice for 3 minutes, which consisted of 10 seconds bursts at 35% power separated by 30 second cooling down intervals. DNA and RNA in the soluble sonicate were further degraded by adding 2 μl Benzonase (Novagen) and incubating at 4° C. for 30 minutes. Cell debris was removed by centrifugation at 20,000 g, 4° C. for 30 minutes.

Inverse phase transition was induced by adding NaCl to the cell lysate at room temperature to achieve a final concentration of 2.0 M, followed by centrifugation at 20,000 g for 15 minutes at room temperature. The resulting pellet contained the respective ELP-AR-LBD fusion protein and non-specifically NaCl precipitated proteins.

The pellet was re-suspended in 40 ml ice-cold 50 mM Hepes pH 7.5, 150 mM NaCl, 0.1% N-Octylglycoside, 10% glycerol, 1 mM DTT and 1 μM DHT and re-centrifuged at 20,000 g, 4° C. for 15 minutes to remove the non-specifically NaCl precipitated proteins. The inverse transition cycle was repeated two additional times to increase the purity of the respective ELP-AR-LBD fusion protein and reduce the final volume to 25 ml.

### Isolation and Purification of Fusion Protein Containing Glucocorticoid Receptor Ligand Binding Domain (GR-LBD)

A single colony of E. coli strain BL21 Star (DE3) containing the ELP-GR-LBD fusion protein was inoculated into 500 ml CircleGrow (Q-BIOgene, San Diego, Calif.) supplemented with 100 μg/ml ampicillin (Sigma) and grown at 37° C. with shaking at 250 rpm for 24 hours. The culture was harvested and suspended in 50 mM Hepes pH 7.5, 150 mM NaCl, 1 mM DTT, 10% glycerol, 0.1% CHAPS and 1 Complete EDTA free Protease inhibitor pellet (Roche, Indianapolis, Ind.). Cells were lysed by ultrasonic disruption on ice for 3 minutes, which consisted of 10 seconds bursts at 35% power separated by 30 second cooling down intervals. DNA and RNA in the soluble lysate were further degraded by adding 2 μl Benzonase (Novagen) and incubating at 4° C. for 30 minutes. Cell debris was removed by centrifugation at 20,000 g, 4° C. for 30 minutes.

Inverse phase transition was induced by adding NaCl to the cell lysate at room temperature to achieve a final concentration of 2.0 M, followed by centrifugation at 20,000 g for 15 minutes at room temperature. The resulting pellet contained the ELP-GR-LBD fusion protein and non-specifically NaCl precipitated proteins.

The pellet was re-suspended in 40 ml ice-cold in 50 mM Hepes pH 7.5, 150 mM NaCl, 1 mM DTT, 10% glycerol, 0.1% CHAPS and re-centrifuged at 20,000 g, 4° C. for 15 minutes to remove the non-specifically NaCl precipitated proteins. The inverse transition cycle was repeated two additional times to increase the purity of the ELP-GR-LBD fusion protein and reduce the final volume to 10 ml.

### Isolation and Purification of Fusion Proteins Containing Estrogen Receptor Ligand Binding Domain (ERα-LBD)

A single colony of E. coli strain BL21 Star (DE3) containing the respective ELP-ERα-LBD fusion protein was inoculated into 500 ml CircleGrow (Q-BIOgene, San Diego, Calif.) supplemented with 100 μg/ml ampicillin (Sigma), 10% sucrose (Sigma) and grown at 27° C. with shaking at 250 rpm for 48 hours. The culture was harvested and suspended in 40 ml 50 mM Tris-HCL pH 8.0, 150 mM KCL, 1 mM EDTA, 1 mM DTT and 1 Complete EDTA free Protease inhibitor pellet (Roche, Indianapolis, Ind.). Cells were lysed by ultrasonic disruption on ice for 3 minutes, which consisted of 10 seconds bursts at 35% power separated by 30 second cooling down intervals. DNA and RNA in the soluble lysate were further degraded by adding 2 μl Benzonase (Novagen) and incubating at 4° C. for 30 minutes. Cell debris was removed by centrifugation at 20,000 g, 4° C. for 30 minutes.

Inverse phase transition was induced by adding NaCl to the cell lysate at room temperature to achieve a final concentration of 1.5 M, followed by centrifugation at 20,000 g for 15 minutes at room temperature. The resulting pellet contained the respective ELP-ERα-LBD fusion protein and non-specifically NaCl precipitated proteins.

The pellet was re-suspended in 40 ml ice-cold 50 mM Tris-HCL pH 8.0, 150 mM KCL, 1 mM EDTA, 1 mM DTT and re-centrifuged at 20,000 g, 4° C. for 15 minutes to remove the non-specifically NaCl precipitated proteins. The inverse transition cycle was repeated two additional times to increase the purity of the respective ELP-ERα-LBD fusion protein and reduce the final volume to 10 ml.

### Isolation and Purification of Fusion Proteins Containing G Protein Alpha Q (Gαq)

A single colony of E. coli strain BL21 Star (DE3) containing the respective ELP-G_{αq} fusion protein was inoculated into 500 ml CircleGrow (Q-BIOgene, San Diego, Calif.) supplemented with 100 μg/ml ampicillin (Sigma) and 1 μM GDP and grown at 37° C. with shaking at 250 rpm for 24 hours. The culture was harvested and suspended in 40 ml 50 mM Hepes pH 7.5, 150 mM NaCl, 1.0% CHAPS, 10% glycerol, 1 mM DTT, 10 μM GDP and 1 Complete EDTA free Protease inhibitor pellet (Roche, Indianapolis, Ind.). Cells were lysed by ultrasonic disruption on ice for 3 minutes, which consisted of 10 seconds bursts at 35% power separated by 30 second cooling down intervals. DNA and RNA in the soluble lysate were further degraded by adding 2 μl Benzonase (Novagen) and incubating at 4° C. for 30 minutes. Cell debris was removed by centrifugation at 20,000 g, 4° C. for 30 minutes.

Inverse phase transition was induced by adding NaCl to the cell lysate at room temperature to achieve a final concentration of 2.0 M, followed by centrifugation at 20,000 g for 15 minutes at room temperature. The resulting pellet contained the respective ELP-G_{αq} fusion protein and non-specifically NaCl precipitated proteins.

The pellet was re-suspended in 30 ml ice-cold 50 mM Hepes pH 7.5, 150 mM NaCl, 1.0% CHAPS, 10% glycerol, 1 mM DTT, 10 μM GDP and re-centrifuged at 20,000 g, 4° C. for 15 minutes to remove the non-specifically NaCl precipitated proteins. The inverse transition cycle was repeated two additional times to increase the purity of the respective ELP-G_{αq} fusion protein and reduce the final volume to 5 ml.

### Isolation and Purification of Fusion Proteins Containing 1-Deoxy-D-Xylulose 5-Phosphate Reductoisomerase (DXR)

A single colony of E. coli strain BL21 Star (DE3) containing the respective ELP-DXR fusion protein was inoculated into 500 ml CircleGrow (Q-BIOgene, San Diego, Calif.) supplemented with 100 μg/ml ampicillin (Sigma), 1 mM MnCl₂(VWR) and grown at 37° C. with shaking at 250 rpm for 24 hours. The culture was harvested and suspended in 40 ml 0.1 M Tris pH 7.6, 1 mM DTT and 1 Complete EDTA free Protease inhibitor pellet (Roche, Indianapolis, Ind.). Cells were lysed by ultrasonic disruption on ice for 3 minutes, which consisted of 10 seconds bursts at 35% power separated by 30 second cooling down intervals. DNA and RNA in the soluble lysate were further degraded by adding 2 μl Benzonase (Novagen) and incubating at 4° C. for 30 minutes. Cell debris was removed by centrifugation at 20,000 g at 4° C. for 30 minutes.

Inverse phase transition was induced by adding NaCl to the cell lysate at room temperature to achieve a final concentration of 2.0 M, followed by centrifugation at 20,000 g for 15 minutes at room temperature. The resulting pellet contained the respective ELP-DXR fusion protein and non-specifically NaCl precipitated proteins.

The pellet was re-suspended in 20 ml ice-cold 0.1 M Tris pH7.6, 1 mM DTT and centrifuged at 20,000 g, 4° C. for 15 minutes to remove the non-specifically NaCl precipitated proteins. The inverse transition cycle was repeated two additional times to increase the purity of the respective ELP-DXR fusion protein and reduce the final volume to 5 ml.

### Isolation and Purification of Fusion Protein Containing G Protein Alpha S (Gαs)

A single colony of E. coli strain BL21 Star (DE3) containing the ELP-G_{αs} fusion protein was inoculated into 500 ml CircleGrow (Q-BIOgene, San Diego, Calif.) supplemented with 100 μg/ml ampicillin (Sigma) and grown at 37° C. with shaking at 250 rpm for 24 hours. The culture was harvested and suspended in 40 ml PBS, 10% glycerol, 1 mM DTT and 1 Complete EDTA free Protease inhibitor pellet (Roche, Indianapolis, Ind.). Cells were lysed by ultrasonic disruption on ice for 3 minutes, which consisted of 10 seconds bursts at 35% power separated by 30 second cooling down intervals. DNA and RNA in the soluble lysate were further degraded by adding 2 μl Benzonase (Novagen) and incubating at 4° C. for 30 minutes. Cell debris was removed by centrifugation at 20,000 g, 4° C. for 30 minutes.

Inverse phase transition was induced by adding NaCl to the cell lysate at room temperature to a final concentration of 1.5 M, followed by centrifugation at 20,000 g for 15 minutes at room temperature. The resulting pellet contained the ELP-G_{αs} fusion protein and non-specifically NaCl precipitated proteins.

The pellet was re-suspended in 10 ml ice-cold PBS, 10% glycerol, 1 mM DTT and centrifuged at 20,000 g, 4° C. for 15 minutes to remove the non-specifically NaCl precipitated proteins. The inverse transition cycle was repeated two additional times to increase the purity of the ELP-G_{αs} fusion protein and reduce the final volume to 1 ml.

### Example 6

### Production of 10 Proteins without Chromatography

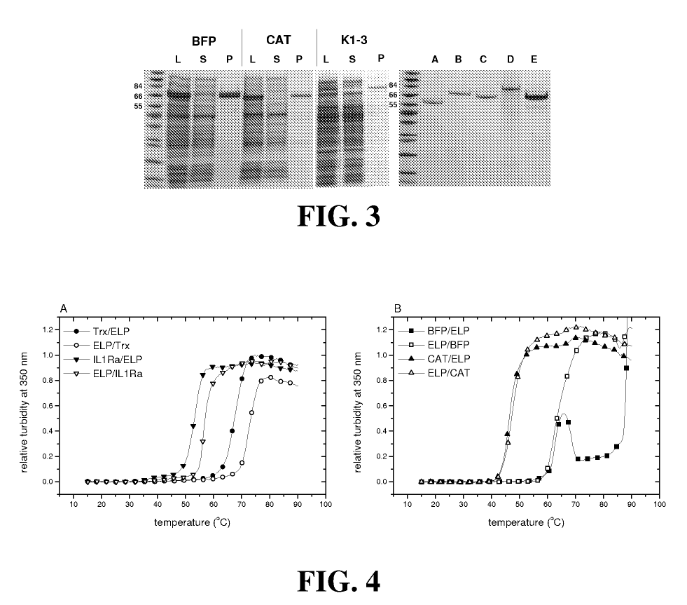

The deltaPhase™ system technology, as set forth above in Example 1, was successfully tested for expression and purification of ten proteins. The results, presented in Table 2 and in the SDS-PAGE, FIG. 3 for three proteins, clearly show that diverse proteins can be purified to >95% purity. Systematic evaluation of ELP fusion protein expression and purification has been performed by having thoroughly characterized blue fluorescent protein (BFP), thioredoxin (Trx), chloramphenicol acetyltransferase (CAT), calmodulin (CalM), and angiostatin (K1-3) expressed as a fusion protein with ELP1 [V₅A₂G₃-90] and with a tag for purification by immobilized metal affinity chromatography (IMAC). Expression was performed in E. coli. Yields obtained for purification of the ELP fusion proteins are listed in Table 2.

TABLE 2
Applications of deltaPhase ™ for Protein Purification

| | MW | Yield | ELP-Protein Fusion |
|---|---|---|---|
| Target Proteins | (kDa)^{a} | (mg/L)^{b} | Activity Confirmed |
| Angiostatin (K1-3) | 30.7 | 27 | Yes |
| Blue fluorescent protein (BFP) | 26.9 | 100 | Yes |
| Calmodulin (CalM) | 16.7 | 75* | Yes |
| Chloramphenicol | 25.7 | 80 | Yes |
| acetyltransferase (CAT) | | | |
| Green fluorescent protein (GFP) | 26.9 | 78 | Yes |
| Interleukin 1 receptor | 17.0 | 8 | Yes |
| antagonist (IL1rRa) | | | |
| Luciferase | 60.8 | 10 | ND** |
| Tissue transglutaminase (tTg) | 77.0 | 36 | Yes |
| Tendamistat | 7.9 | 22 | Yes |
| Thioredoxin (Trx) | 11.7 | 50 | Yes |

Table 2. ELP1 Fusion Protein Sequences Synthesized with the deltaPhase ™ System.
^{a}denotes the average molecular weight of the protein.
^{b}Purified yields indicate the best yield of target protein derived from ELP fusion.
**ND = not determined

FIG. 3 shows an SDS-PAGE gel of ITC purification of BFP, CAT, and K1-3. The figure includes the soluble E. coli lysate (L), the supernatant following centrifugation above the Tₜ of the fusion protein (S), and the purified protein (P). The second gel shows purified ELP[V₅A₂G₃-90] fusions of Trx (A), BFP (B), CAT (C), K1-3 (D), GFP (E).

### Example 7

### Production of 10 Pharmaceutically Relevant Peptides

Using the deltaPhase™ system, as set forth above in Example 1, 10 pharmaceutically relevant peptides ranging in size from 2.0 to 6.2 kDa and ranging in isoelectric points from 4.11-12.3 were expressed and purified. After extensive work varying multiple expression and purification conditions, 6 of the peptides with greater than 90% purity and yields of 17-23 mg per liter were successfully expressed and purified, as set forth in Table 3 below.

TABLE 3

| | Amount of Fusion | Amount of Peptide | |
|---|---|---|---|
| | Produced | Produced | |
| Peptide | Mg/L | Mg/L | Purity |
| Morphine Modulating | 224 | 17 | 99% |
| Neuropeptide (MMN) | | | |
| Neuropeptide Y (NPY) | 222 | 20 | 98% |
| Orexin B | 320 | 19 | 91% |
| Leptin | 415 | 19 | 97% |
| ACTH | 133 | 19 | 99% |
| Calcitonin | 260 | 23 | 98% |

Fusion proteins generated included: ELP4-60-MMN, ELP4-60-NPY, ELP4-60-Orexin B, ELP4-60-Leptin, ELP4-60-ACTH, ELP4-60-GH and ELP1-90-Calcitonin.

Four of the peptides proved more challenging to produce in substantial quantities. This is not surprising, given the variable nature of peptides, including size, solubility and propensity of proteolysis. Fusion proteins of the challenging peptides, ELP-adrenomedullin (AM), ELP-Parathyroid Hormone (PTH), ELP-Defensin, and ELP-growth hormone were successfully produced. However, after cleavage of the ELP from the peptide of interest, either the cleavage system with tobacco etch virus (TEV) was inadequate, or the peptide was insoluble. Only partial cleavage of ELP-growth hormone was achieved, and no peptide remained after cleavage of ELP-AM, ELP-PTH, and ELP-Defensin. These results prove the flexibility and wide-ranging application of the ELP system for the purification of therapeutically relevant peptides without chromatography.

### Example 8

### Fusion Protein Activity

Fusion peptide therapeutic proteins were generated using the following four proteins: blue fluorescent protein (BFP), chloramphenicol acetyltransferase (CAT), thioredoxin (Trx), and interleukin 1 receptor antagonist (IL-Ra). Each composition was generated in both an ELP/protein and a protein/ELP orientation, utilizing ELP1 [V₅A₂G₃-90].

### Linkers of the Eight Fusion Constructs:

(SEQ ID NO: 59)- ELP

| | |
|---|---|
| CAT/ELP | CAT - VENLYFQGGMG |

(SEQ ID NO: 60)- CAT

| | |
|---|---|
| ELP/CAT | ELP - VPGWPSSGDYDIPTTENLYFQGAH |

(SEQ ID NO: 61)- ELP

| | |
|---|---|
| Trx/ELP | Trx - GSGSGHMHHHHHHSSGLVPRGSGK |

(SEQ ID NO: 62)- Trx

| | |
|---|---|
| ELP/Trx | ELP - VPGWPSSGDYDIPTTENLYFQGAH |

(SEQ ID NO: 63)- ELP

| | |
|---|---|
| BFP/ELP | BFP - VDKLAAALDMHHHHHHSSGLVPRGSGK |

(SEQ ID NO: 64)- BFP

| | |
|---|---|
| ELP/BFP | ELP - VPGWPSSGDYDIPTTENLYFQGAH |

(SEQ ID NO: 65)- ELP

| | |
|---|---|
| IL-1Ra/ELP | IL-1Ra - LENLYFQGGMG |

(SEQ ID NO: 66)- IL-1Ra

| | |
|---|---|
| ELP/IL-1Ra | ELP - VPGWPSSGDYDIPTTENLYFQGAH |

All eight protein fusion constructs have been transformed into BLR(DE3) cells, grown in triplicate in 50 mL TB media, and purified by ITC. During one round of ITC the phase transition is induced by adding NaCl to lower Tₜ and the large, micron-sized aggregates are collected by centrifugation. The pellets are resuspended in low ionic strength buffer followed by a cold spin to remove insoluble contaminants trapped in the ELP fusion protein pellet. Each fusion construct has been cycled through the phase transitions 3-5 times to obtain pure protein.

The yields of the protein/ELP fusions was higher than those of the ELP/protein constructs for all constructs, however the ratio between the yields in the two orientations depend on the size of the target protein (Table 4). The yields obtained for the smaller proteins Trx and IL-1Ra are significantly higher than those for the larger proteins CAT and BFP in the ELP/protein direction.

TABLE 4
Yields, specific activities, and transition
temperatures of the eight fusion proteins

| | Yield* | Specific | |
|---|---|---|---|
| Fusion protein | (mg/L culture) | activity** | T₁ (° C.)*** |
| BFP/ELP | 79 ± 15 | 1704 ± 293 | 62.9 ± 0.3 |
| | | | 67.9 ± 0.5 |
| ELP/BFP | 0.5 ± 0.06 | 1620 ± 111 | 62.4 ± 0.5 |
| CAT/ELP | 39 ± 7 | 8058 ± 1437 | 46.1 ± 0.3 |
| ELP/CAT | 2.2 ± 2.1 | 2984 ± 1783 | 47.1 ± 0.2 |
| Trx/ELP | 87 ± 4 | 116.6 ± 9.9 | 67.3 ± 0.4 |
| ELP/Trx | 27 ± 9 | 68.6 ± 18.0 | 72.9 ± 0.4 |
| IL-1Ra/ELP | 15.8 ± 4.8 | 2.0 ± 0.4 | 53.1 ± 0.4 |
| ELP/IL-1Ra | 8.2 ± 1.3 | 0.5 ± 0.2 | 55.9 ± 0.6 |

*The yields have been extrapolated from 50 mL cultures to 1 L.
**for Trx and CAT the specific activity is measured in U/mg, one unit corresponds to the conversion of 1 nmole substrate per minute. The specific activity for BFP is reported as the integrated area obtained by fluorescence per mg protein (A.U./μg), and the activity for IL-1Ra is measured as the EC50 value in μg/mL.
***All fusion protein concentrations are 2 μM and the experiments are carried out in PBS buffer.
No significant changes in activity are observed for Trx/ELP and BFP/ELP compared to the free un-fused target protein (Trabbic-Carlson K, et al. Protein Eng. Des. Sel. 2004, 17: 57-66; Meyer D E, Chilkoti A, Nat. Biotechnol. 1999, 17: 1112-1115), whereas the CAT/ELP shows a small decrease in activity of about 15% compared to free CAT. Previously it is found that the IL-1Ra/ELP activity is decreased more than 100 fold compared to the free IL-1Ra which is the largest difference observed for these ELP fusion proteins (Shamji, Setton et al., accepted, in press).

The activity of Trx in the two fusion constructs have been measured by the insulin reduction assay as described by Holmgren (I l. Holmgren A., J. Biol. Chem. 1979, 254:9627-9632; Holmgren A., Bjornstedt M., Methods Enzymol. 1984, 107:295-300). In the net enzymatic reaction the disulfide bonds in insulin are reduced while NADPH is oxidized to NADP⁺ which is followed spectroscopically at 340 nm. The initial rates are measured in each experiment at 25° C. and converted into specific activities. The assay has been carried out three times for each of the three purified batches. The specific activities in U/mg fusion protein of the two fusion constructs are shown in Table 4 (1 U in the Trx assay is the conversion of 1 nmole substrate per minute). Differences in specific activity between the two Trx constructs have been observed; the specific activity of ELP/Trx is reduced to about 60% of the Trx/ELP activity.

The activity of CAT fused to the ELP in the two different orientations has been determined by enzymatic acetylation of the substrate 1-deoxychloramphenicol. The activity has been measured on each of the three purifications in triplicate. The remaining substrate and the formed product are separated by thin layer chromatography before measuring the fluorescence intensity of both. The specific activities of the two CAT constructs are reported in U/mg in Table 4 where 1 U is the conversion of 1 nmole substrate per minute. Here it is seen that the specific activity of the ELP/CAT construct is reduced compared to CAT/ELP. A significant reduction is observed and only about 37% of the activity remains in the ELP/CAT fusion protein (Table 4).

I-L1Ra competes with interleukin 1 (IL-1) for the interleukin 1 receptor and the potency of the antagonist is measured by a cell proliferation assay where active IL-1Ra inhibit the growth of the cells. Human peripheral blood leukocytes RPMI 1788 have been grown for 72 hours with and without the presence of IL-1Ra either in the form of ELP fusions or un-fused, commercially available antagonist. The proliferation has been measured by the CellTiter Glo assay. The activities of the two fusion constructs are listed in Table 4. Like CAT and Trx, IL-1Ra also show a decrease in activity in the ELP/protein orientation and IL-1Ra/ELP is four times more potent than ELP/IL-1Ra. Comparing to un-fused IL-1Ra the free IL-1Ra is about 300 times more active than IL-1Ra/ELP (the EC50 for IL-1Ra is 1.6 ng/ml).

BFP is not a biologically active protein but fluoresces in the near-UV region. Fluorescence is a sensitive measurement of changes in the tertiary structure of a protein and here it is used to evaluate structural differences between the two BFP fusion constructs. Fluorescence spectra of each BFP construct have been collected from 430 to 600 nm after excitation at 385 nm. The curves were integrated and the area normalized with protein mass. The results are listed in Table 4. The ELP/BFP used in these experiments has been grown up from two 1 L cultures in order to obtain concentrations in the same range as BFP/ELP for the fluorescence measurements. After normalizing with protein mass no significant difference is observed in fluorescence between the two BFP constructs.

The transition temperature (Tₜ) for fusion proteins is sensitive to the hydrophobic/hydrophilic ratio of the accessible surface area. The ELP/protein constructs are not as active as in the opposite fusions, except for BFP constructs, and if that decrease in activity is due to major structural changes the transition temperature will shift. The change in optical density of each construct has been followed from 15 to 90° C. at 350 nm and Tₜ was derived as the mid-point of the transition (FIG. 4 and Table 4). The concentration of each fusion protein was 2 μM, which was chosen due to the very low yields of some of the ELP/protein constructs. FIG. 4 shows the increase in turbidity as a function of temperature of 2 μM of each of the fusion constructs in PBS buffer: A. Trx/ELP (closed circles), ELP/Trx (open circles), IL-1Ra/ELP (closed down triangles), and ELP/IL-1Ra(open down triangles) and B. BFP/ELP (closed squares), ELP/BFP (open squares), CAT/ELP (closed up triangles), ELP/CAT (open up triangles). Tₜ is calculated as the mid-point of each transition curve and shown in Table 4.

The transition temperatures for ELP/Trx and ELP/IL-1Ra are larger than their protein/ELP counterparts. Trx and IL-1Ra constructs differ 5.6° C. and 2.8° C., respectively, whereas the difference between the two CAT constructs is almost negligible (FIGS. 4A and B, Table 4). The ELP/BFP show one transition and form large aggregates at 62.4° C. whereas the BFP/ELP construct show a very different pattern; this fusion protein starts out forming aggregates at almost the same temperature as the ELP/BFP protein but as the temperature increases the aggregates dissociate and instead the BFP/ELP construct forms micelle-like structures. The transition temperature for the micelle-like structure formation is also reported as the mid-point of the curve and shown in Table 4 as the second transition temperature for BFP/ELP.

All eight constructs were purified by inverse transition cycling where the fusion proteins have been cycled through an aggregated phase induced by adding NaCl followed by a centrifugation step and finally the obtained pellets have been resuspended in buffer. The final yields of ELP/protein fusions after the purification process are lower compared to their respective protein/ELP constructs however smaller target proteins have higher relative yields. The lower yields are not due to significant losses during purification but a result of lower expression levels of the ELP/proteins most likely due to misfolding of the target proteins during translation. The purified ELP/proteins are assumed to fold somewhat differently from the native fold of the target protein; the specific activities are all lower in the ELP/protein orientation, except for BFP. In addition the measured transition temperatures are slightly higher for the ELP/protein constructs compared to the protein/ELP constructs, again except for BFP. The transition temperature depends on the hydrophobic/hydrophilic ratio of the fused protein indicating that the ELP/protein constructs are folded but not in a native fold.

### Example 9

### Half-Life of ELP1

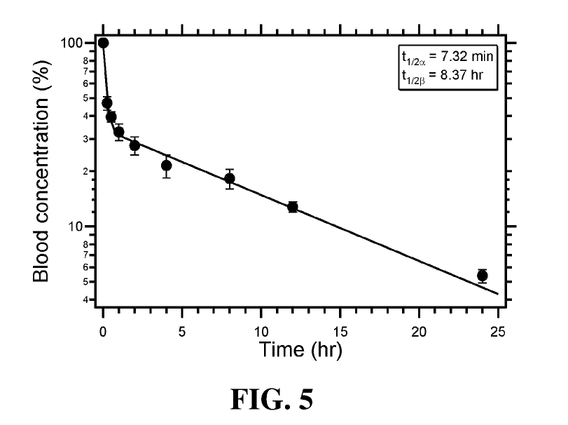

The pharmacokinetics of ELP1 were determined by intravenously administering [¹⁴C]ELP1 to nude mice (Balb/c nu/nu) bearing a leg/flank FaDu xenograft and collecting blood samples at various time intervals after administration. The blood concentration time-course and plasma half-lives (initial t_{1/2α} and terminal t_{1/2β}) are shown in FIG. 5. The blood pharmacokinetics exhibited a characteristic distribution and elimination response for macromolecules, which was well described by a bi-exponential process.

The plasma concentration time-course curve in FIG. 5 was fit to the analytical solution of a two-compartment model to approximate both an elimination and distribution response (shown as the solid line in FIG. 5) and the relevant pharmacokinetic parameters are shown in Table 5. The distribution volume of the ELP (1.338 μl) was nearly identical to the hypothetical plasma volume of 1.363 μl (Barbee, R. W., et al., Am. J. Physio. 263(3) (1992) R728-R733), indicating that the ELP did not rapidly distribute or bind to specific organs and tissues directly after administration. The AUC is a measure of the cumulative exposure to ELP in the central compartment or the blood plasma. The body clearance is defined as the rate of ELP elimination in the body relative to its plasma concentration and is the summation of clearance through all organs including the kidney, liver and others. These pharmacokinetic parameters, such as a long terminal half-life (t_{1/2β}=8.37 hr) and low distribution volume (i.e., nearly equal to the plasma volume), are considered favorable for the delivery of therapeutics to solid tumors and potentially other disease sites. This is because such values indicate that the ELP has properties suitable for exploiting the EPR effect in a fashion similar to that seen for other successful drug carriers (R. Duncan, Nat. Rev. Drug. Discov. 2(5) (2003) 347-360).

TABLE 5
Pharmacokinetic parameters calculated for [¹⁴C]ELP1

| | | | | | AUC | |
|---|---|---|---|---|---|---|
| | k₁ | k₂ | kₑ | V_{d} | (mg ELP | Cl_{B} |
| | (hr⁻¹) | (hr⁻¹) | (hr⁻¹) | (μL) | hr/mL | (μL/hr) |
| ELP1-150 | 3.54 | 1.99 | 0.24 | 1,338 | 7.1 | 317 |

The mass transfer rate constants are from a standard two-compartment model (k₁, from central to peripheral compartment; k₂, from peripheral to central compartment; and kₑ, elimination from central compartment). The distribution volume (V_{d}), central compartment concentration time-course area under the curve (AUC) and body clearance (Cl_{B}) are displayed. Data are shown as the mean values (n=5, except V_{d} and initial plasma concentration (Cₒ) was calculated from a similar cohort with n=3).

### Example 10

### Biodistribution of ELPs in Nude Mice

¹⁴C Labeled ELP1-150 and/or ¹⁴C Labeled ELP2-160

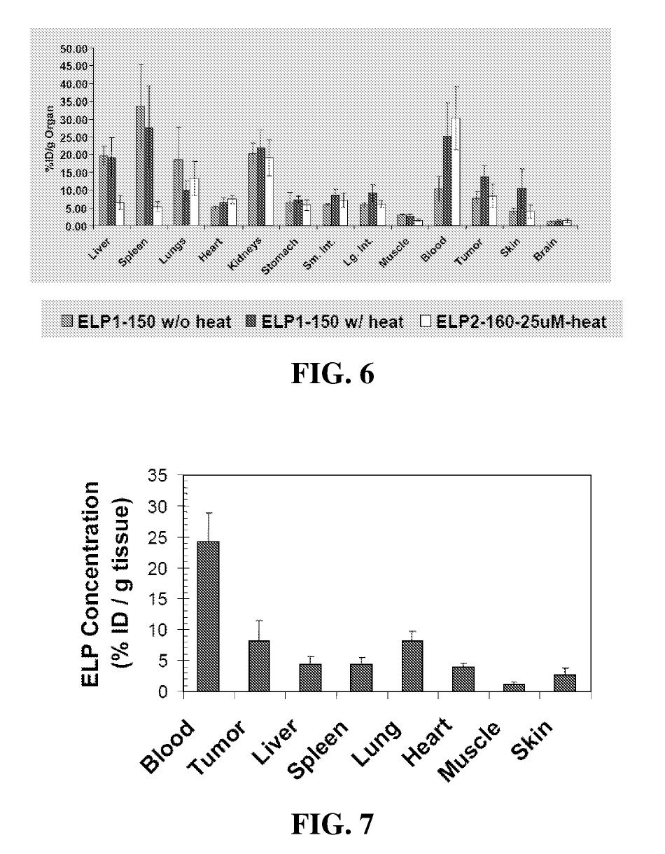

¹⁴C labeled ELP1-150 and/or ¹⁴C labeled ELP2-160 were administered to nude mice with a FaDu tumor (mean±SD, n=6). The tumor was heated post administration of the ELP in a water bath at 41.5° C. As can be seen in FIG. 6, the distribution is highest to the organs with the highest blood content: liver, kidneys, spleen, and lungs.

### ¹⁴C Labeled ELP2-[V₁A₈G₇-160]

¹⁴C labeled ELP2-[V₁A₈G₂-160] (T₁>60° C.) was administered to nude mice for a plasma concentration of 15 μM. ELP concentrations were determined following 1 hour of heating (41° C.) of an implanted FaDu tumor, located in the right hind leg of the nude mouse. Data are shown as the mean, plus the 95% confidence interval. N=6.

Results are shown in FIG. 7, in the graph of percent injected dose (ID) per gram (g) of tissue vs. tissue type. ELP concentration was measured 1.5 hours following systemic administration of ¹⁴C labeled ELP2-[V₁A₈G₇-160]. The highest distribution is seen in organs with the highest blood content: liver, kidneys, spleen, and lungs.

While the invention has been has been described herein in reference to specific aspects, features and illustrative embodiments of the invention, it will be appreciated that the utility of the invention is not thus limited, but rather extends to and encompasses numerous other variations, modifications and alternative embodiments, as will suggest themselves to those of ordinary skill in the field of the present invention, based on the disclosure herein.

Correspondingly, the invention as hereinafter claimed is intended to be broadly construed and interpreted, as including all such variations, modifications and alternative embodiments, within its spirit and scope.

1.-22. (canceled)

23. A therapeutic composition comprising a fusion protein and a pharmaceutically-acceptable carrier,
wherein the fusion protein comprises GLP-1 and at least one elastin-like protein (ELP) component, and wherein the GLP-1 exhibits an extended half-life in circulation as compared to its unfused counterpart.

24. The therapeutic composition of claim 23, wherein the ELP component is constructed of one or more peptide repeat units defined by SEQ ID NOS: 1-12.

25. The therapeutic composition of claim 24, wherein the ELP component comprises repeats of VPGXG, IPGXG, and/or LPGXG, where X is a genetically-encoded amino acid.

26. The therapeutic composition of claim 25, wherein the ELP component comprises VPGXG repeats, wherein each X is independently selected from V, A, and G, or is independently selected from K, V, and F.

27. The therapeutic composition of claim 26, wherein X is V, A, and G in the ratio of about V5, A2, and G3.

28. The therapeutic composition of claim 27, wherein the ELP component comprises at least 60 repeating units of VPGXG.

29. The therapeutic composition of claim 26, wherein X is K, V, and F in the ratio of about K1, V2, and F1.

30. The therapeutic composition of claim 29, wherein the ELP component comprises at least 60 repeating units of VPGXG.

31. The therapeutic composition of claim 26, wherein each X is V.

32. The The therapeutic composition of claim 31, wherein the ELP component comprises at least 60 repeating units of VPGXG.

33. The therapeutic composition of claim 23, wherein the ELP component is at the C-terminus of GLP-1.

34. The therapeutic composition of claim 23, further comprising a spacer sequence between GLP-1 and the ELP component.

35. The therapeutic composition of claim 23, wherein the composition is formulated for parenteral administration.

36. The therapeutic composition of claim 35, wherein the composition is formulated for subcutaneous, intramuscular, or intravenous administration.

37. A method of treating a subject in need of GLP-1, including administering to the patient a therapeutically effective amount of the composition of claim 23.

38. The method of claim 37, wherein said subject is a human subject.

39. The method of claim 37, wherein said composition is formulated for subcutaneous administration.
